(19) United States
(12) Patent Application Publication
(10) Pub. No.: US 2025/0011402 A1
(43) Pub. Date: Jan. 9, 2025

(54) ANTIBODIES AGAINST DISEASE CAUSING AGENTS OF POULTRY AND USES THEREOF

(71) Applicant: NOVOBIND LIVESTOCK THERAPEUTICS INC., Vancouver (CA)

(72) Inventors: Hamlet ABNOUSI, Vancouver (CA); Slade Andrew LOUTET, Vancouver (CA); Filip VAN PETEGEM, Vancouver (CA); Tsz Ying Silvia CHEUNG, Vancouver (CA)

(73) Assignee: NOVOBIND LIVESTOCK THERAPEUTICS INC., Vancouver (CA)

(21) Appl. No.: 18/777,457

(22) Filed: Jul. 18, 2024

Related U.S. Application Data
(63) Continuation-in-part of application No. 17/141,052, filed on Jan. 4, 2021.
Application No. 17/141,052 is a continuation of application No. PCT/IB2019/001198, filed on Jul. 3, 2019.
(60) Provisional application No. 62/694,164, filed on Jul. 5, 2018.
(60) Provisional application No. 63/534,432, filed on Aug. 24, 2023.

(51) Int. Cl. C07K 16/12; A61K 39/00; A61K 45/06; A61P 31/04

(52) U.S. Cl. CPC C07K 16/1282; A61K 45/06; A61P 31/04; A61K 2039/505; C07K 2317/565; C07K 2317/569; C07K 2317/76

(57) ABSTRACT

Described are methods and antibodies useful for reducing, eliminating, or preventing infection with a bacterial population in an animal. Also described herein are antigens useful for targeting by heavy chain antibodies and VHH fragments for reducing a bacterial population in an animal.

### REFERENCE TO RELATED APPLICATIONS

This application is a continuation-in-part patent application of U.S. patent application Ser. No. 17/141,052, filed on Jan. 4, 2021 claiming benefit to and priority to United States Provisional Application Ser. No. 62/694,164, filed on Jul. 5, 2018, and to International Patent Application No. PCT/IB2019/001198, filed on Jul. 3, 2019, each of which are hereby incorporated by this reference in their entireties.

### REFERENCE TO ELECTRONIC SEQUENCE LISTING

The application contains a Sequence Listing which has been submitted electronically in .XML format and is hereby incorporated by reference in its entirety. Said .XML copy, created on Sep. 12, 2024, is named “068964.001USCIP.xml” and is 2,292,563 bytes in size. The sequence listing contained in this .XML file is part of the specification and is hereby incorporated by reference herein in its entirety.

### FIELD OF THE INVENTION

This invention relates to methods and compositions for the control of microorganisms associated with necrotic enteritis and uses thereof.

### BACKGROUND OF THE INVENTION

Losses to the agriculture industry following contamination of livestock with pathogens are a global burden. With a growing global population and no significant increase in the amount of farmland available to agriculture, there is a need to produce larger quantities of food without using more space. Traditional treatment of animals with antibiotics is a major contributor to the emergence of multi-drug resistant organisms and is widely recognised as an unsustainable solution to controlling contamination of livestock. There is a need for the development of pathogen-specific molecules that inhibit infection or association of the pathogen with the host, without encouraging resistance. Global losses to the poultry industry due to the pathogenic organisms that cause necrotic enteritis has been estimated to be $6 billion⁽¹⁾ USD per annum. The bacterium Clostridium perfringens is the causative agent of necrotic enteritis in poultry in conjunction with a variety of predisposing factors⁽²⁾.

### SUMMARY OF THE INVENTION

With reference to the definitions set out below, described herein are polypeptides comprising heavy chain variable region fragments (V_{H}Hs) whose intended use includes but is not limited to the following applications in agriculture or an unrelated field: diagnostics, in vitro assays, feed, therapeutics, substrate identification, nutritional supplementation, bioscientific and medical research, and companion diagnostics. Also described herein are polypeptides comprising V_{H}Hs that bind and decrease the virulence of disease-causing agents in agriculture. Further to these descriptions, set out below are the uses of polypeptides that comprise V_{H}Hs in methods of reducing transmission and severity of disease in host animals, including their use as an ingredient in a product. Further described are the means to produce, characterise, refine, and modify V_{H}Hs for this purpose.

In accordance with the purpose(s) of this invention, as embodied and broadly described herein, this invention, in one aspect, relates to a polypeptide comprising at least one variable region fragment of a heavy chain antibody (V_{H}H) having an amino acid sequence at least 99% identical to any one of SEQ ID NOs: 1 to 56 or 212 to 340 or 695 to 968 or 1791 to 1795, wherein the polypeptide is capable of a) reducing the cytotoxicity of NetB with an IC50 value less than 1 mM by at least 90%; b) reducing the binding of CnaA to collagen by greater than 50% at 2 uM; or c) reducing Cpa lecithinase activity by greater than 40% at 1 uM.

In one embodiment, the V_{H}H comprises: a complementarity determining region 1 (CDR1) as set forth in any one of SEQ ID NOs: 57 to 106 or 341 to 458 or 969 to 1242 or 1796 to 1800, a complementarity determining region 2 (CDR2) as set forth in any one of SEQ ID NOs: 107 to 156 or 459 to 576 or 1243 to 1516 or 1801 to 1805, and a complementarity determining region 3 (CDR3) as set forth in any one of SEQ ID NOs: 157 to 206 or 577 to 694 or 1517 to 1790 or 1806 to 1810.

In another embodiment, the polypeptide comprises a plurality of V_{H}Hs. In some embodiments, the plurality of V_{H}Hs is identical to another V_{H}H of the plurality of V_{H}Hs. In other embodiments, the plurality of V_{H}Hs comprises at least three V_{H}Hs. In additional embodiments, the plurality of V_{H}Hs are covalently coupled to one another by a linker, the linker comprising one or more amino acids.

In another aspect, the invention relates to a polypeptide complex comprising the polypeptides as disclosed herein, wherein the polypeptide complex comprises: a first component polypeptide, and a second component polypeptide, wherein the first component polypeptide and the second component polypeptide are not covalently linked together and are coupled together by a protein-protein interaction, a small molecule-protein interaction, or a small molecule-small molecule interaction, and wherein each of the first and the second component polypeptides comprise a V_{H}H which specifically binds a pathogen.

In one embodiment, the pathogen is a poultry-associated bacterium. In particular embodiments, wherein the poultry-associated bacterium comprises a species of Clostridium. And in other embodiments, the species of Clostridium is Clostridium perfringens.

In other embodiments, the V_{H}H specifically binds a Clostridium virulence factor, wherein the Clostridium virulence factor is NetB polypeptide, NetB-like toxin polypeptide, Cpa polypeptide, Cpa-like toxin polypeptide, Cpb2 polypeptide, Cpb2-like toxin polypeptide, CnaA polypeptide, CnaA-like polypeptide, CnaA collagen binding domain polypeptide, or CnaA collagen binding domain-like polypeptide. In particular embodiments, the V_{H}H specifically binds an antigen or polypeptide at least 99% identical to SEQ ID NOs: 207, 208, 209, 210, 211 or combinations thereof.

In additional embodiments, the polypeptides disclosed herein further comprise a vitamin, an antibiotic, a hormone, an antimicrobial peptide, a steroid, a probiotic, a probiotic, a bacteriophage, chitin, chitosan, B-1,3-glucan, vegetable extracts, peptone, shrimp meal, krill, algae, B-cyclodextran, alginate, gum, tragacanth, pectin, gelatin, an additive spray, a toxin binder, a short chain fatty acid, a medium chain fatty acid, yeast, a yeast extract, sugar, a digestive enzyme, a digestive compound, an essential mineral, an essential salt, or fiber.

In other aspects, the invention relates to a vector or a cell comprising a nucleic acid or a plurality of nucleic acids encoding the polypeptide disclosed herein, wherein the polypeptide is produced by incubating the cell in a medium suitable for secretion of the polypeptide from the cell and purifying the polypeptide from the medium. In one embodiment, the cell is a yeast selected from the genus Pichia or Saccharomyces, or a bacterial cells selected from the genus Escherichia or a probiotic bacterium, wherein the probiotic bacteria is selected from the group consisting of the genus Bacillus, the genus Lactobacillus, the genus Bifidobacterium.

In yet another aspect, the invention relates to a method of of reducing the activity of a Clostridium perfringens virulence factor comprising administering to a poultry animal, a non-poultry animal species, or human individual a polypeptide at least 99% identical to SEQ IDs Nos: 207, 208, 209, 210, 211, or combinations thereof, wherein the method reduces or prevents a poultry-associated bacterial infection or reduces transmission or prevents transmission of the poultry-associated bacterial from the poultry species to another poultry animal, another animal species, or human individual.

In one embodiment, the poultry animal is a species of a chicken, turkey, duck, quail, pigeon, squab, ostrich, or goose and the non-poultry animal species is a pig, sheep, goat, horse, cow, llama, alpaca, mink, rabbit, dog, cat, or human.

In another embodiment, the polypeptide is adapted for introduction to the alimentary canal orally or rectally, provided to the exterior surface (for example, as a spray or submersion), provided to the medium in which the animal dwells (including air based media), provided by injection, provided intravenously, provided via the respiratory system, provided via diffusion, provided via absorption by the endothelium or epithelium, or provided via a secondary organism such as a yeast, bacterium, algae, bacteriophages, plants and insects to a host.

In one another embodiment, the polypeptides of SEQ ID NOs: 210 or 211 can outcompete Clostridium perfringens surface-expressed CnaA binding to collagen by 10%, 20%, 30%, 40%, 50%, 60%, 70%, 80%, 90%, 95%, 99%, or 100%.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a schematic of camelid heavy chain only antibodies and their relationship to V_{H}H domains. FIG. 1B illustrates the framework regions (FRs) and complementarity determining regions (CDRs) of the V_{H}H domain.

FIGS. 2A-20 show phage ELISA binding data for V_{H}H antibodies of this disclosure.

FIG. 3 shows that unlabeled CnaA can outcompete labeled CnaA for collagen binding.

FIG. 4A shows the inhibition of NetB cytotoxicity by four VHH antibodies of this disclosure. FIG. 4B shows the inhibition of CnaA binding to collagen by two V_{H}H antibodies of this disclosure. FIG. 4C shows the stability of six V_{H}H antibodies of this disclosure in a chicken jejunal extract.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the present invention, the following terminology is used in accordance with the definitions below.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

### A. Host

As referred to herein, “host”, “host organism”, “recipient animal”, “host animal” and variations thereof refer to the intended recipient of the product when the product constitutes a feed. In certain embodiments, the host is from the superorder Galloanserae. In certain embodiments, the host is a poultry animal. In certain embodiments, the poultry animal is a chicken, turkey, duck, quail, pigeon, squab or goose. In certain embodiments, the poultry animal is a chicken.

### B. Pathogens

As referred to herein, “pathogen”, “pathogenic”, and variations thereof refer to virulent microorganisms, that can be associated with host organisms, that give rise to a symptom or set of symptoms in that organism that are not present in uninfected host organisms, including the reduction in ability to survive, thrive, reproduce. Without limitation, pathogens encompass parasites, bacteria, viruses, prions, protists, fungi, and algae. In certain embodiments, the pathogen is a bacterium belonging to the Clostridium genus.

“Virulence”, “virulent” and variations thereof refer to a pathogen's ability to cause symptoms in a host organism. “Virulence factor” refers to nucleic acids, plasmids, genomic islands, genes, peptides, proteins, toxins, lipids, macromolecular machineries, or complexes thereof that have a demonstrated or putative role in infection.

“Disease-causing agent” refers to a microorganism, pathogen, or virulence factor with a demonstrated or putative role in infection.

### C. Bacteria

As referred to herein, “bacteria”, “bacterial” and variations thereof refer, without limitation, to Clostridium species, or any other bacterial species associated with host organisms. In certain embodiments, bacteria may not be virulent in all host organisms it is associated with.

### D. Antibodies

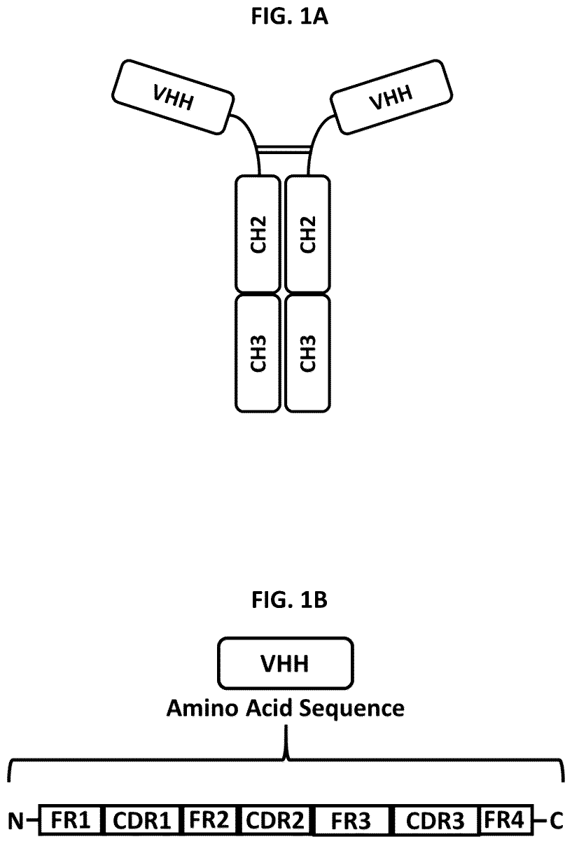

A schematic of camelid heavy chain only antibodies and their relationship to V_{H}H domains and complementarity determining regions (CDRs) is shown in FIG. 1. (Panel A). A camelid heavy chain only antibody consists of two heavy chains linked by a disulphide bridge. Each heavy chain contains two constant immunoglobulin domains (CH2 and CH3) linked through a hinge region to a variable immunoglobulin domain (V_{H}H). (Panel B) are derived from single V_{H}H domains. Each V_{H}H domain contains an amino acid sequence of approximately 110-130 amino acids. The V_{H}H domain consists of the following regions starting at the N-terminus (N): framework region 1 (FR1), complementarity-determining region 1 (CDR1), framework region 2 (FR2), complementarity-determining region 2 (CDR2), framework region 3 (FR3), complementarity-determining region 3 (CDR3), and framework region 4 (FR4). The domain ends at the C-terminus (C). The complementarity-determining regions are highly variable, determine antigen binding by the antibody, and are held together in a scaffold by the framework regions of the V_{H}H domain. The framework regions consist of more conserved amino acid sequences; however, some variability exists in these regions.

As referred to herein “VHH” refers to an antibody or antibody fragment comprising a single heavy chain variable region which may be derived from natural or synthetic sources. NBXs referred to herein are an example of a VHH. In a certain aspect a V_{H}H may lack a portion of a heavy chain constant region (CH2 or CH3), or an entire heavy chain constant region. As referred to herein, NBXs are a V_{H}H that binds NETB.

As referred to herein “heavy chain antibody” refers to an antibody that comprises two heavy chains and lacks the two light chains normally found in a conventional antibody. The heavy chain antibody may originate from a species of the Camelidae family or Chondrichthyes class. Heavy chain antibodies retain specific binding to an antigen in the absence of any light chain.

As referred to herein “specific binding”, “specifically binds” or variations thereof refer to binding that occurs between an antibody and its target molecule that is mediated by at least one complementarity determining region (CDR) of the antibody's variable region. Binding that is between the constant region and another molecule, such as Protein A or G, for example, does not constitute specific binding.

As referred to herein “antibody fragment” refers to any portion of a conventional or heavy chain antibody that retains a capacity to specifically bind a target antigen and may include a single chain antibody, a variable region fragment of a heavy chain antibody, a nanobody, a polypeptide or an immunoglobulin new antigen receptor (IgNAR).

As referred to herein an “antibody originates from a species” when any of the CDR regions of the antibody were raised in an animal of said species. Antibodies that are raised in a certain species and then optimized by an in vitro method (e.g., phage display) are considered to have originated from that species.

As referred to herein “conventional antibody” refers to any full-sized immunoglobulin that comprises two heavy chain molecules and two light chain molecules joined together by a disulfide bond. In certain embodiments, the antibodies, compositions, feeds, products, and methods described herein do not utilize conventional antibodies.

### E. Production System

As referred to herein, “production system” and variations thereof refer to any system that can be used to produce any physical embodiment of the invention or modified forms of the invention. Without limitation, this includes but is not limited to biological production by any of the following: bacteria, yeast, algae, arthropods, arthropod cells, annelids, plants, mammalian cells. Without limitation, biological production can give rise to antibodies that can be intracellular, periplasmic, membrane-associated, secreted, or phage-associated. Without limitation, “production system” and variations thereof also include, without limitation, any synthetic production system. This includes, without limitation, de novo protein synthesis, protein synthesis in the presence of cell extracts, protein synthesis in the presence of purified enzymes, and any other alternative protein synthesis system.

### F. Product

As referred to herein, “product” refers to any physical embodiment of the invention or modified forms of the invention, wherein the binding of the V_{H}H to any molecule, including itself, defines its use. Without limitation, this includes a feed, a feed additive, a nutritional supplement, a premix, a medicine, a therapeutic, a drug, a diagnostic tool, a component or entirety of an in vitro assay, a component or the entirety of a diagnostic assay (including companion diagnostic assays).

### G. Feed Product

As referred to herein, “feed product” refers to any physical embodiment of the invention or modified forms of the invention, wherein the binding of the VHH to any molecule, including itself, defines its intended use as a product that is taken up by a host organism. Without limitation, this includes a feed, a pellet, a feed additive, a nutritional supplement, a premix, a medicine, a therapeutic or a drug.

### II. Detailed Description of the Invention

Descriptions of the invention provided are to be interpreted in conjunction with the definitions and caveats provided herein.

For many years, the agriculture industry has utilized antibiotics to control pathogenic bacteria. These antibiotics also acted as growth promoters. This approach has contributed greatly to the spread of antibiotic resistance amongst pathogenic organisms. To phase out antibiotics for non-medicinal purposes and limit antimicrobial resistance, the use of antibiotics as growth promoters in animal feed has already been banned in Europe (effective from 2006). Widespread protection of farmed animals through vaccination has failed due to the short lifespan of many agriculturally important animals, logistical challenges with vaccination of industrial-sized flocks, and high costs. The withdrawal of prophylactic antibiotics in animal feed and the failure of vaccination to offer widespread protection underpins the need for the development of non-antibiotic products to administer to agricultural animals to prevent infection and promote growth.

Significant pathogens affecting poultry animals include bacteria, such as members of the Clostridium and Salmonella genera, among others, as well as parasites, such as members of the Eimeria genus.

Losses due to Clostridium perfringens, the causative agent of necrotic enteritis are estimated at $6 billion⁽¹⁾ USD per annum. Necrotic enteritis can lead to significant mortality in chicken flocks⁽³⁾. At subclinical levels, damage to the intestinal mucosa caused by C. perfringens leads to decreased digestion and absorption, reduced weight gain and increased feed conversion ratio⁽³⁾. Typically, necrotic enteritis occurs after some other predisposing factor causes mucosal damage to the chicken⁽²⁾ C. perfringens virulence factors associated with necrotic enteritis have been shown to include production of toxins and adherence to collagen⁽⁴⁾.

Subclinical infection by Eimeria parasites is one of the most common predisposing factors for necrotic enteritis⁽²⁾. These parasites can physically damage the epithelial layer and induce mucose generation⁽⁵⁾. In addition, Eimeria parasites are also the causative agent of coccidiosis in chickens, a disease that is estimated to cause €10 billion in poultry losses globally⁽⁶⁾. Coccidiosis is characterized by reduced weight gain and feed conversion, malabsorption, cell lysis of cells linking, and diarrhea⁽⁷⁾.

Changes to the gastrointestinal tract microbiota can also serve to induce necrotic enteritis. For example, early infections early of chicks by Salmonella enterica can result in the development of necrotic enteritis in experimental models, possibly through alteration of the host immune response⁽⁸⁾.

Other proposed predisposing factors for the development of necrotic enteritis include immune suppression by viral infections, physical changes to the gut caused by alterations to the diet, and poor animal husbandry⁽²⁾.

Earlier efforts in the field of this invention rely on the host organism to generate protection against disease-causing agents. This approach is often limited by the short lifespan of the host organisms affected by the pathogens listed above, which do allow the host organism's immune system enough time to generate long-lasting immunity. Furthermore, the effectiveness of prior arts is limited by technical challenges associated with widespread vaccination of large flocks of host organisms. These problems are circumvented by introducing exogenous peptides that neutralise the virulence and spread of the disease-causing agent into the host via feed without eliciting the host immune response. Moreover, the methods described herein provide scope for the adaptation and refinement of neutralising peptides, which provides synthetic functionality beyond what the host is naturally able to produce.

Antibody heavy chain variable region fragments (V_{H}Hs) are small (12-15 kDa) proteins that comprise specific binding regions to antigens. When introduced into an animal, V_{H}Hs bind and neutralise the effect of disease-causing agents in situ. Owing to their smaller mass, they are less susceptible than conventional antibodies, such as previously documented IgYs, to cleavage by enzymes found in host organisms, more resilient to temperature and pH changes, more soluble, have low systemic absorption and are easier to recombinantly produce on a large scale, making them more suitable for use in animal therapeutics than conventional antibodies.

### A. Antibodies for Preventing or Reducing Virulence (Summary)

In one aspect, the present invention provides a polypeptide or pluralities thereof comprising a V_{H}H or V_{H}Hs that bind disease-causing agents to reduce the severity and transmission of disease between and across species. In certain embodiments, the V_{H}H is supplied to host animals. In certain embodiments, the VHH is an ingredient of a product.

### 1. Binding to Reduce Virulence

In another aspect, the present invention provides a polypeptide or pluralities thereof comprising a V_{H}H or V_{H}Hs that bind disease-causing agents, and in doing so, reduce the ability of the disease-causing agent to exert a pathological function or contribute to a disease phenotype. In certain embodiments, binding of the VHH(s) to the disease-causing agent reduces the rate of replication of the disease-causing agent. In certain embodiments, binding of the VHH(s) to the disease-causing agent reduces the ability of the disease-causing agent to bind to its cognate receptor. In certain embodiments, binding of the VHH(s) to the disease-causing agent reduces the ability of the disease-causing agent to interact with another molecule or molecules. In certain embodiments, binding of the VHH(s) to the disease-causing agent reduces the mobility or motility of the disease-causing agent. In certain embodiments, binding of the VHH(s) to the disease-causing agent reduces the ability of the disease-causing agent to reach the site of infection. In certain embodiments, binding of the VHH(s) to the disease-causing agent reduces the ability of the disease-causing agent to cause cell death.

2. Antibodies Derived from Llamas

In a further aspect, the present invention provides a method for the inoculation of Camelid or other species with recombinant virulence factors, the retrieval of mRNA encoding V_{H}H domains from lymphocytes of the inoculated organism, the reverse transcription of mRNA encoding V_{H}H domains to produce cDNA, the cloning of cDNA into a suitable vector and the recombinant expression of the V_{H}H from the vector. In certain embodiments, the camelid can be a dromedary, camel, llama, alpaca, vicuna or guacano, without limitation. In certain embodiments, the inoculated species can be, without limitation, any organism that can produce single domain antibodies, including cartilaginous fish, such as a member of the Chondrichthyes class of organisms, which includes for example sharks, rays, skates, and sawfish. In certain embodiments, the heavy chain antibody comprises a sequence set forth in Table 1. In certain embodiments, the heavy chain antibody comprises an amino acid sequence with at least 80%, 90%, 95%, 97%, or 99% identity to any sequence disclosed in Table 1. In certain embodiments, the heavy chain antibody possess a CDR1 set forth in Table 2. In certain embodiments, the heavy chain antibody possess a CDR2 set forth in Table 2. In certain embodiments, the heavy chain antibody possess a CDR3 set forth in Table 2.

TABLE 1
Unique SEQ ID NOs for V_{H}H antibodies
of this disclosure

| SEQ | | | |
|---|---|---|---|
| ID | | | Anti- |
| NO: | NBX | Amino acid sequence | gen |
| 1 | NBX0301 | QVQLQESGGGVVQAGGSLSLSCSPY | NetB |
| | | QRASSLFAMGWFRQSPGKEREFVAG | |
| | | ISWNGDKSQYADSVKDRFTISRDND | |
| | | KNTVFLQMNSLKPEDTAVYYCAAHR | |
| | | ASFELGFATHDYDFWGQGTQVTVSS | |
| 2 | NBX0302 | QVQLQESGGGLVQTGGSLRLSCVAS | NetB |
| | | GSIFSISSAVWSRQAPGKQREWVAS | |
| | | IFSDGSTNYATSVKGRFTISRDHAK | |
| | | NTVYLQMNSLKPEDTGVYYCAVDGY | |
| | | RGQGTQVTVSS | |
| 3 | NBX0303 | QVQLQESGGGLVQAGGSLRLSCTAS | NetB |
| | | GRTLSYWTMGWFRQAPGKEREFVAA | |
| | | INWSSGTRYSDSVRDRFTIDGDTDK | |
| | | TTVYLEMNKMNLDDSAVYYCAAHRA | |
| | | SFGLGYQTHEYDFWGQGTQVTVSS | |
| 4 | NBX0304 | QVQLQESGGGLVQTGDSLRLSCTAS | NetB |
| | | GGTFSSYTMGWYRQAPGKGREFVGS | |
| | | ITWNSEVTYYADSVKGRFTISRDNA | |
| | | KNMMNLQMNSLKPEDTAVYYCAAGR | |
| | | AGSGFTSWGQGTQVTVSS | |
| 5 | NBX0305 | QVQLQESGGGLVQPGGSLRLSCTAS | NetB |
| | | GFTLDKYAVGWFRQAPGKEREGVSC | |
| | | ISSIDDSTDYVDSVKGRFTISRDNA | |
| | | KNAVYLQMNSLKPEDTAVYNCMTIP | |
| | | LPYGSTCDIPSRSDLLAINYWGKGT | |
| | | LVTVSS | |
| 6 | NBX0306 | QVQLQQSGGGLVQPGGSLRLSCTAS | NetB |
| | | GFTVPYYYIGWFRQAPGKEREGISC | |
| | | IASSSGKAYYADSVKGRFTLSKDNA | |
| | | KNTAYLQMDSLKPEDTAVYYCAALR | |
| | | KYGSTCYLHVLEYDYWGQGTQVNVS | |
| | | S | |
| 7 | NBX0307 | QVQLQESGGGLVQAGGSLRLSCTAS | NetB |
| | | GRTLSYWTMGWFRQVPGKEREFVAA | |
| | | INWSSGTRYSESVRDRFTIDGDTDK | |
| | | TTVYLEMNKMNLDDSAVYYCAAHRA | |
| | | SFGLGYQTHEYDFWGQGTQVTVSS | |
| 8 | NBX0308 | QVQLQQSGGGLVQAGGSLRLSCTAS | NetB |
| | | GRTLSYWTMGWFRQVPGKEREFVAA | |
| | | INWSSGTRYSESVRDRFTIDGDTDK | |
| | | TTVYLEMNKMNLDDSAVYYCAAHRA | |
| | | SFGLGYQTHEYDFWGQGTQVTVSS | |
| 9 | NBX0309 | QVQLQQSGGGLVQAGGSLRLSCAAS | NetB |
| | | GSTFNNYMIGWFRQAPGKEREFVAT | |
| | | ISGSGAGTFYADSVRGRFTISRDNA | |
| | | KNTVYLQMNSLKLEDTAGYYCARRM | |
| | | SRSGIFGLRDYDSWGQGTQVTVSS | |
| 10 | NBX0310 | QVQLQQSGGGVVQAGGSLSLSCSPY | NetB |
| | | QRASSLFAMGWFRQSPGKEREFVAG | |
| | | ISWNGDKSQYADSVKDRFTISRDND | |
| | | KNTVFLQMNSLKPEDTAVYYCAAHR | |
| | | ASFELGFATHDYDFWGQGTQVTVSS | |
| 11 | NBX0311 | QVQLQESGGGLVQAGGSLRLSCAAS | NetB |
| | | GRTFSNADMAWFRQSPGKERESVAA | |
| | | ISWSGGRTYYADSVKGRATISRDIA | |
| | | KDTVYLQMNSLKPEDTAVYYCAAGG | |
| | | YSNLPTSYGYWGQGTQVTVSS | |
| 12 | NBX0316 | QVQLQESGGGLVQTGGSLRLSCAAS | CnaA |
| | | GRAFSTYGMGWFRQAPGKEREFVAG | |
| | | ISSSGAGSAYVDSVKHRFTVSRDNA | |
| | | KNTMYLQMNSLKPEDTAVYYCAAST | |
| | | TSWGKFAHYIYWGQGTQVTVSS | |
| 13 | NBX0317 | QVQLQESGGGLVQAGGSLRLSCAAS | CnaA |
| | | GGTFSSYIMGWFRQAPGKDREFVGA | |
| | | ISWSGGVTHYADSVKGRFTISRDNA | |
| | | KNTVYLQMNSLKPEDTAVYYCAADS | |
| | | RISAGGSYYEADFGSWGQGTQVTVS | |
| | | S | |
| 14 | NBX0318 | QVQLQESGGGLVQAGGSLRLSCVVS | NetB |
| | | GSIMSIRVMGWYRQAPGKQRELVAT | |
| | | MSRGNTINYADSVRGRFTISRDNAK | |
| | | STVYLQMNSLKPEDTDVYYCAALLD | |
| | | SYYWGQGTQVTVSS | |
| 15 | NBX0319 | QVQLQESGGGLVQAGGSLRLSCAAS | NetB |
| | | ASIISIRVMGWYRQAPGKQRELVAT | |
| | | MSRGGTINYADSVRGRFTISRDNAK | |
| | | STVFLEMNSLKPEDTAVYYCTALLD | |
| | | SYYWGQGTQVTVSS | |
| 16 | NBX0320 | QVQLQESGGGLVQAGGSLRLSCAAS | NetB |
| | | GSIFSIRVMGWYRQAPGKQRELVAT | |
| | | MSRGGTINYADSVRGRFTISRDNAK | |
| | | ITVYLQMTSLKPEDTAVYYCAALLD | |
| | | SYYWGQGTQVTVSS | |
| 17 | NBX0321 | QVQLQESGGGLVQPGGSLRLSCTAS | NetB |
| | | GFTLDKYAVGWFRQAPGKEREGVSC | |
| | | ISSIDDSTDYVDSVKGRFTISRDNA | |
| | | KNAVYLQMNSLKPEDTAVYDCMTIP | |
| | | LPYGSTCRIPSRSDLLAINYWGKGT | |
| | | LVTVSS | |
| 18 | NBX0322 | QVQLQESGGGLVQAGGSLRLSCQGS | NetB |
| | | GRTFSTYAMGWYRQAPGKEREFVAA | |
| | | ITRGGNTIYADSVKGRFTISRVSDK | |
| | | NTVYLQMSSLKPEDTAVYYCAADRI | |
| | | IVPRDPMDYWGKGTLVTVSS | |
| 19 | NBX0323 | QVQLQQSGGGLVQAGGSLRLSCTAS | NetB |
| | | GRTLSYWTMGWFRQAPGKEREFVAA | |
| | | INWSSGTRYSDSVRDRFTIDGDTDK | |
| | | TTVYLEMNKMNLDDSAVYYCAAHRA | |
| | | SFGLGYQTHEYDFWGQGTQVTVSS | |
| 20 | NBX0324 | QVQLQESGGGLVQAGGSLRLTCTAS | NetB |
| | | GRTLSYWTMGWFRQAPGKEREFVAA | |
| | | INWSSGTRYSDSVKDRFTIDGDSDK | |
| | | TTVYLQMNSLNLDDSAVYYCAAHRA | |
| | | SFGLGYQTHEYDFWGQGTQVTVSS | |
| 21 | NBX0325 | QVQLQESGGGLVQAGDSLRLSCLAS | CnaA |
| | | GGTFSSYIMGWFRQAPGKDREFVGA | |
| | | ISWSGGVTHYADSVKGRFTISRDNA | |
| | | KNTVYLQMNSLKPEDTAVYYCAADS | |
| | | RISAGGSYYEADFGSWGQGTQVTVS | |
| | | S | |
| 22 | NBX0326 | QVQLQESGGGLVQAGGSLRLTCAVS | CnaA |
| | | GRTFSAIHMGWFRQAPGKEREFVAG | |
| | | ISWSGGGTAYGGTVKGRFTISRDNA | |
| | | KNTVSLQMNSLKSEDTAVYYCAASD | |
| | | TDWGRSASYDYWGQGTQVTVSA | |
| 23 | NBX0327 | QVQLQQSGGGLVQAGGSLRLSCAAS | CnaA |
| | | GGTFSSYVMGWFRQAPGKDREFVGA | |
| | | ISWSGGVTHYADSVKGRFTISRDNA | |
| | | KNTVYLQMNSLKPEDTAVYYCAADS | |
| | | RISAGGSYYEADFGSWGQGTQVTVS | |
| | | S | |
| 24 | NBX0328 | QVQLQQSGGGLVQAGDSLRLSCATS | Cpa |
| | | GRTFSSYTMGWFRQTPGKEREFVAA | |
| | | ISWSGTYYTDSVKGRFTISRDTAKN | |
| | | TVYLQMNSLKPEDTAVYYCAVGSRR | |
| | | LYYSSDINYWGQGTQVTVSS | |
| 25 | NBX0329 | QVQLQESGGGLVQAGGSLRLSCATS | Cpa |
| | | GLTVSRYTMGWFRQTPGKDREFVAA | |
| | | ISWSGTYYTDSVKGRFTISVDNAKN | |
| | | MVYLQMNSLKPEDTAVYYCAAGSRR | |
| | | LYYSNDINYWGQGTQVTVSS | |
| 26 | NBX0330 | QVQLQESGGGLVQAGGSLRLSCAAS | Cpa |
| | | SRTFSNYAMAWFRQTPGKEREFLAT | |
| | | INGDTTFTIYADSVKGRFTISRDNA | |
| | | KNTLYLQMNSLKAEDTAVYYCAARQ | |
| | | WNPTMRERDYGYWGQGTEVTVSS | |
| 27 | NBX0331 | QVQLQESGGGLVQAGGSLRLSCAAS | Cpb2 |
| | | GRVFENYFMGWFRQAPGKEREFVAA | |
| | | TNWNTATNWNTYYAAFVKARFTISR | |
| | | DKAKNTLYLQMNSLKPEDTAVYYCA | |
| | | ATGSRTYDVVDYYDYWGQGTQVTVS | |
| | | S | |
| 28 | NBX0332 | QVQLQESGGGLVQAGGSLRLSCAAS | Cpb2 |
| | | GRTFSSYSMAWFRQAPGKERESVAA | |
| | | ITYSGITTAYTDSVKGRFTIWRDNA | |
| | | KNTVYLQMNSLKPEDTAVYYCAASY | |
| | | SASRSYPFGEYDYWGQGTQVTVSS | |
| 29 | NBX0333 | QVQLQESGGGLVQAGGSLRLSCAAS | Cpb2 |
| | | GRTFSSYSMAWFRQAPGKERESVAA | |
| | | ITYSGISTAYTDSVKGRFTISRDNA | |
| | | KNTVYLYMNSLKPEDTAVYYCAASY | |
| | | SASRSYPFGEYDYWGQGTQVTVSS | |
| 30 | NBX0334 | QVQLQESGGGLVQPGGSLRLSCAAS | Cpb2 |
| | | GFTFSNSAMSWMRQAPGKAVEWVSS | |
| | | INIGGDSRRYAESVAGRFTISRDNA | |
| | | KNTLYLQMNSLKPEDTAVYYCAKGL | |
| | | ASTIRGQGTQVTVSS | |
| 31 | NBX0335 | QVQLQESGGGLVQPGGSLRLSCAAS | Cpb2 |
| | | GFTFSNSAMSWMRQAPGKGVEWVSS | |
| | | IEVGGGRRYAESVAGRFTISRDNAK | |
| | | NTLYLQMNSLKPEDTAVYYCSKGLA | |
| | | STIRGQGTQVTVSS | |
| 32 | NBX0336 | QVQLQESGGGLVQPGGSLRLSCAAS | Cpb2 |
| | | GFTFSNSAMSWMRQAPGKGVEWVSS | |
| | | IGIDGGRRYAEAVAGRFTISRDNAK | |
| | | NTLYLQMNSLKPEDTAVYYCAKGLA | |
| | | STIRGQGTQVTVSS | |
| 33 | NBX0337 | QVQLQESGGGLVQPGGSLRLSCAAS | Cpb2 |
| | | GFTFSNSAMSWMRQAPGKGVEWVSS | |
| | | IGIGGGTTRYADSVAGRFTISRDNA | |
| | | KNTLYLQMNSLKPEDTAVYYCAKGL | |
| | | ASTIRGQGTQVTVSS | |
| 34 | NBX0338 | QVQLQESGGGLVQAGDSLRLSCATS | Cpa |
| | | GRSFSSYTMGWFRQTPGKEREFVAA | |
| | | ISWSGTYYTDSVKGRFTISRDNAKN | |
| | | TVYLQMNSLKPEDTAVYYCAVGSRR | |
| | | LYYSSDINYWGQGTQVTVSS | |
| 35 | NBX0339 | QVQLQESGGGLVQAGDSLRLSCATS | Cpa |
| | | GLTVSRYTMGWFRQTPGKEREFVAA | |
| | | ISWSGTYYTDSVKGRFTISRDNAKN | |
| | | MVYLQMNSLKPEDTAVYYCAAGSRR | |
| | | LHYSSDINYWGQGTQVTVSS | |
| 36 | NBX0340 | QVQLQESGGGLVQAGESLRLSCLAA | Cpa |
| | | GRTFSTSTLGWFRQAPGLEREFVAA | |
| | | IRYTSDYTARTTDYADSVKGRFAIS | |
| | | RDYIKQAVYLQMNNLKPEDTAVYYC | |
| | | AAAKYGMGYSDPSGYTYWGQGTQVT | |
| | | VSS | |
| 37 | NBX0341 | QVQLQESGGGLVQAGGSLRLSCAAS | Cpa |
| | | SRTFSNYAMAWFRQTPGKEREFLAA | |
| | | ITGDTAFTIYADSVKGRFTISRDNP | |
| | | KNTLYLQMNSLKAEDTAVYYCAARQ | |
| | | WNPTMRERDYGYWGQGTEVTVSS | |
| 38 | NBX0342 | QVQLQESGGGLVQAGGSLRLSCAAS | Cpb2 |
| | | GRRFRLYHMGWFRQAPGKEREFVAV | |
| | | ISWSGGTTVYADSVKGRFTISRDNE | |
| | | KNAGYLQMNSLKPEDTAVYYCAVDR | |
| | | LIESFSDPTAWPRMDYWGKGALVTV | |
| | | SS | |
| 39 | NBX0343 | QVQLQESGGGLVQPGGSLRLSCAAS | Cpb2 |
| | | GFTFSNSAMSWMRQAPGKGVEWVSS | |
| | | INIGGGTTSYADSVAGRFTISRDNA | |
| | | KNTLYLQMNSLKPEDTAVYYCAKGL | |
| | | ASTIRGQGTQVTVSS | |
| 40 | NBX0344 | QVQLQESGGGLVQPGGSLRLSCAAS | Cpb2 |
| | | GFTFSNSAMSWMRQAPGKGVEWVSS | |
| | | INIGGGTRRYAESVAGRFTISRDNA | |
| | | KNTLYLQMNSLKPEDTAVYYCAKGL | |
| | | ASTIRGQGTQVTVSS | |
| 41 | NBX0345 | QVQLQESGGGLVQAGGSLRLSCAAS | Cpb2 |
| | | GRKFRLYHMGWFRQAPGKEREFVAV | |
| | | ISWSGGSTVYADSVKGRFTISRDNE | |
| | | KNAGYLQMNSLKPEDTAVYYCAVDR | |
| | | LIESFSDPTAWPRMDYWGKGALVTV | |
| | | SS | |
| 42 | NBX0346 | QVQLQQSGGGLVQPGGSLRLSCAAS | Cpb2 |
| | | GFTFSNSAMSWMRQAPGKGVEWVSS | |
| | | INIGGGTRYADSVAGRFTIYRDNAK | |
| | | NTLYLQMNSLKSEDTAVYYCAKGLA | |
| | | STIRGQGTQVTVSS | |
| 43 | NBX0347 | QVQLQESGGGSVQAGGSLRLSCAAS | Cpb2 |
| | | GRTFSSYDMGWFRQAPGKEREWVAS | |
| | | ISYNIYYADFVKGRFTISKDNAKNT | |
| | | VSLQMNSLKPEDTAVYYCAAVQRRG | |
| | | SYSYDRAQSYDYWGQGTQVTVSS | |
| 44 | NBX0348 | QVQLQESGGGLVQPGGSLRLSCAAS | Cpb2 |
| | | GFTFSNSAMSWMRQAPGKGVEWVSS | |
| | | IEIGGTRRYAESVAGRFTISRDNAK | |
| | | NTLYLQMNSLKAEDTAVYYCAKGLA | |
| | | STIRGQGTQVTVSS | |
| 45 | NBX0349 | QVQLQESGGGLVQPGGSLRLSCAAS | Cpb2 |
| | | GFTFSNSPMSWMRQAPGKGVEWVSS | |
| | | INIGAGTTRYAESVAGRFTIARDNA | |
| | | KNTLYLQMNSLKPEDTAVYYCAKGL | |
| | | ASTIRGQGTQVIVSS | |
| 46 | NBX0350 | QVQLQESGGGLVQPGGSLRLSCAAS | Cpb2 |
| | | GFTFSNSAMSWMRQAPGKGVEWVSS | |
| | | INIGGGDKRYAESVAGRFTISRDNA | |
| | | KNTLYLQMNSLKFEDTAVYYCAKGL | |
| | | ASTIRGQGTQVTVSS | |
| 47 | NBX0351 | QVQLQESGGGLVQPGGSLRLSCAAS | Cpb2 |
| | | GFTFSNSAMSWMRQAPGKGVEWVSS | |
| | | IETGGTKRYAESVAGRFTISRDNAK | |
| | | NTLNLQMNSLKPEDTAVYYCAKGLA | |
| | | STIRGQGTQVTVSS | |
| 48 | NBX0352 | QVQLQQSGGGLVQPGGSLRLSCAAS | Cpb2 |
| | | GFTFSNSPMSWMRQAPGKGVEWVSS | |
| | | INIGEGTTRYAESVAGRFTISRDNV | |
| | | KNTLYLQMNSLKPEDTAVYYCAKGL | |
| | | ASTIRGQGTQVTVSS | |
| 49 | NBX0353 | QVQLQESGGGLVQPGGSLRLSCAAS | Cpb2 |
| | | GFTFSNSPMSWMRQAPGKGVEWVSS | |
| | | INIGGDTRRYAESVAGRFTISRDNA | |
| | | KNTLYLQMNSLKSEDTAVYYCAKGL | |
| | | ASTIRGQGTQVTVSS | |
| 50 | NBX0354 | QVQLQESGGGLVQPGGSLRLSCAAS | Cpb2 |
| | | GFTFSNSAMSWMRQAPGNGVEWVSS | |
| | | VNIDGGRRYAEAVAGRFTISRDNAK | |
| | | NTLYLQMNSLKPEDTAVYYCAKGLA | |
| | | STIRGQGTQVTVSS | |
| 51 | NBX0355 | QVQLQESGGGLVQPGGSLRLSCAAS | Cpb2 |
| | | GFTFSNSAMAWMRQAPGKGVEWVSS | |
| | | ISIDGGRRYAEAVAGRFTISRDNAK | |
| | | NTLYLQMNSLKPEDTAVYYCAKGLA | |
| | | STIRGQGTQVTVSS | |
| 52 | NBX0356 | QVQLQESGGGLVQAGGSLRLSCAAS | Cpb2 |
| | | GGKFTLYHMGWFRQTPGKEREFVAV | |
| | | ISWSGRSTVYADSVKGRFTISRDND | |
| | | KNAGYLQMNSLKPEDTAIYYCAVDR | |
| | | LIEKFSDPTAWPRMDSWGRGTLVTV | |
| | | SS | |
| 53 | NBX0357 | QVQLQESGGGLVQAGDSLRLSCAAS | Cpb2 |
| | | GRTASMGWFRQAPGTQREFVATITR | |
| | | SSIYTDYSDSVKGRFAISRDNAKNT | |
| | | VYLQMNSLKPEDTAVYYCAADSTMS | |
| | | GSSRYSSDYAYWGQGTQVTVSS | |
| 54 | NBX0358 | QVQLQESGGGLVQPGGSLRLSCAAS | Cpb2 |
| | | GFTFSNSPMSWMRQAPGKGVEWVSS | |
| | | IDIGGNRRYAEAVAGRFTISRDNAK | |
| | | NTLYLQMNSLKPEDTAVYYCAKGLA | |
| | | STIRGQGTQVTVSS | |
| 55 | NBX0359 | QVQLQESGGGLVQAGGSLRLSCAVS | Cpb2 |
| | | GRRFTLYHMGWFRQRPGKEREFVAV | |
| | | ISWSGGSTVYADSVKGRFTISRDNE | |
| | | KNAGYLQMNSLKPEDTAVYYCAVDR | |
| | | LIESFSDPTAWPRMDYWGKGALVTV | |
| | | SS | |
| 56 | NBX0360 | QVQLQQSGGGLVQAGGSLRLSCAAS | Cpb2 |
| | | GRRFSLYHMGWFRQAPGKEREFVAV | |
| | | ISWSGGTTVYADSVKGRFTISRDNE | |
| | | KNAGYLQMNSLKPEDTAVYYCAVDR | |
| | | LIESFSDPTAWPRMDYWGKGALVTV | |
| | | SS | |
| 212 | NBX0361 | QVQLQESGGGLVQAGGSLRLSCAAS | NetB |
| | | GSIFSIRVMGWYRQAPGKQRELVAT | |
| | | MSRGGTINYADSVRGRFTISRDNAK | |
| | | STVYLQMNSLKPEDTAVYYCAALLD | |
| | | SYYWGQGTQVTVSS | |
| 213 | NBX0362 | QVQLQESGGGLVQAGGSLRLSCVVS | NetB |
| | | GSIMSIRVMGWYRQAPGKQRELVAT | |
| | | MSRGNTINYADSVRGRFTISRDNAK | |
| | | STVFLEMNSLKPEDTAVYYCAALLD | |
| | | SYYWGQGTQVTVSS | |
| 214 | NBX0363 | QVQLQESGGGLVQAGGSLRLSCAAS | NetB |
| | | ASIISIRVMGWYRQAPGKQRELVAT | |
| | | MSRGGTINYADSVRGRFTISRDNAK | |
| | | STVYLQMNSLKPEDTDVYYCAALLD | |
| | | SYYWGQGTQVTVSS | |
| 215 | NBX0364 | QVQLQESGGGLVQAGGSLRLSCVVS | NetB |
| | | GSIMSIRVMGWYRQAPGKQRELVAT | |
| | | MSRGGTINYADSVRGRFTISRDNAK | |
| | | STVYLQMNSLKPEDTAVYYCTALLD | |
| | | SYYWGQGTQVTVSS | |
| 216 | NBX0365 | QVQLQESGGGLVQAGGSLRLSCAAS | NetB |
| | | GSIFSIRVMGWYRQAPGKQRELVAT | |
| | | MSRGGTINYADSVRGRFTISRDNAK | |
| | | STVYLQMNSLKPEDTAVYYCTALLD | |
| | | SYYWGQGTQVTVSS | |
| 217 | NBX0366 | QVQLQESGGGLVQAGGSLRLSCVVS | NetB |
| | | GSIMSIRVMGWYRQAPGKQRELVAT | |
| | | MSRGGTINYADSVRGRFTISRDNAK | |
| | | NTVYLQMTSLKPEDTAVYYCTALLD | |
| | | SYYWGQGTQVTVSS | |
| 218 | NBX0367 | QVQLQESGGGLVQAGGSLRLSCAAS | NetB |
| | | GSIFSIRVMGWYRQAPGKQRELVAT | |
| | | MSRGGTINYADSVRGRFTISRDNAK | |
| | | STVYLQMNSLKPEDTDVYYCAALLD | |
| | | SYYWGQGTQVTVSS | |
| 219 | NBX0368 | QVQLQESGGGLVQAGGSLRLSCVVS | NetB |
| | | GSIMSIRVMGWYRQAPGKQRELVAT | |
| | | MSRGNTINYADSVRGRFTISRDNAK | |
| | | NTVYLQMTSLKPEDTAVYYCAALLD | |
| | | SYYWGQGTQVTVSS | |
| 220 | NBX0369 | QVQLQESGGGLVQAGGSLRLSCAAS | NetB |
| | | ASIFSIRVMGWYRQAPGKQRELVAT | |
| | | MSRGNTINYADSVRGRFTISRDNAK | |
| | | STVYLQMTSLKPEDTAVYYCAALLD | |
| | | SYYWGQGTQVTVSS | |
| 221 | NBX0370 | QVQLQESGGGLVQAGGSLRLSCVVS | NetB |
| | | GSIMSIRVMGWYRQAPGKQRELVAT | |
| | | MSRGGTINYADSVRGRFTISRDNAK | |
| | | STVYLQMNSLKPEDTAVYYCAALLD | |
| | | SYYWGQGTQVTVSS | |
| 222 | NBX0371 | QVQLQESGGGLVQAGGSLRLSCAAS | NetB |
| | | ASIISIRVMGWYRQAPGKQRELVAT | |
| | | MSRGGTINYADSVRGRFTISRDNAK | |
| | | STVYLQMNSLKPEDTAVYYCTALLD | |
| | | SYYWGQGTQVTVSS | |
| 223 | NBX0372 | QVQLQESGGGLVQAGGSLRLSCAAS | NetB |
| | | ASIISIRVMGWYRQAPGKQRELVAT | |
| | | MSRGGTINYADSVRGRFTISRDNAK | |
| | | STVYLQMNSLKPEDTAVYYCAALLD | |
| | | SYYWGQGTQVTVSS | |
| 224 | NBX0373 | QVQLQESGGGLVQAGGSLRLSCAAS | NetB |
| | | ASIMSIRVMGWYRQAPGKQRELVAT | |
| | | MSRGNTINYADSVRGRFTISRDNAK | |
| | | STVFLEMNSLKPEDTAVYYCAALLD | |
| | | SYYWGQGTQVTVSS | |
| 225 | NBX0374 | QVQLQESGGGLVQAGGSLRLSCAAS | NetB |
| | | ASIMSIRVMGWYRQAPGKQRELVAT | |
| | | MSRGGTINYADSVRGRFTISRDNAK | |
| | | STVYLQMNSLKPEDTDVYYCAALLD | |
| | | SYYWGQGTQVTVSS | |
| 226 | NBX0375 | QVQLQESGGGLVQAGGSLRLSCVVS | NetB |
| | | GSIMSIRVMGWYRQAPGKQRELVAT | |
| | | MSRGGTINYADSVRGRFTISRDNAK | |
| | | STVYLQMNSLKPEDTDVYYCAALLD | |
| | | SYYWGQGTQVTVSS | |
| 227 | NBX0376 | QVQLQESGGGLVQAGGSLRLSCAAS | NetB |
| | | ASIISIRVMGWYRQAPGKQRELVAT | |
| | | MSRGGTINYADSVRGRFTISRDNAK | |
| | | STVYLQMTSLKPEDTAVYYCAALLD | |
| | | SYYWGQGTQVTVSS | |
| 228 | NBX0377 | QVQLQESGGGLVQAGGSLRLSCVVS | NetB |
| | | GSIMSIRVMGWYRQAPGKQRELVAT | |
| | | MSRGNTINYADSVKGRFTISRDNAK | |
| | | STVFLQMNSLKPEDTDVYYCAALLD | |
| | | SYYWGQGTQVTVSS | |
| 229 | NBX0378 | QVQLQESGGGLVQAGGSLRLSCAAS | NetB |
| | | ASIMSIRVMGWYRQAPGKQRELVAT | |
| | | MSRGNTINYADSVRGRFTISRDNAK | |
| | | STVYLQMNSLKPEDTAVYYCAALLD | |
| | | SYYWGQGTQVTVSS | |
| 230 | NBX0379 | QVQLQESGGGLVQAGGSLRLSCVVS | NetB |
| | | GSIMSIRVMGWYRQAPGKQRELVAT | |
| | | MSRGNTINYADSVRGRFTISRDNAK | |
| | | STVYLQMNSLKPEDTAVYYCAALLD | |
| | | SYYWGQGTQVTVSS | |
| 231 | NBX0380 | QVQLQESGGGLVQAGGSLRLSCVVS | NetB |
| | | GSIISIRVMGWYRQAPGKQRELVAT | |
| | | MSRGGTINYADSVRGRFTISRDNAK | |
| | | STVFLEMNSLKPEDTAVYYCAALLD | |
| | | SYYWGQGTQVTVSS | |
| 232 | NBX0381 | QVQLQESGGGLVQAGGSLRLSCAAS | NetB |
| | | ASIISIRVMGWYRQAPGKQRELVAT | |
| | | MSRGNTINYADSVRGRFTISRDDAK | |
| | | NTVYLQMNSLRPDDTAVYYCAALLD | |
| | | SYYWGQGTQVTVSS | |
| 233 | NBX0501 | QVQLQESGGGLVQAGGSLRLSCAAS | NetB |
| | | GSIFSINVMGWYRQAPGKQRDLVAL | |
| | | ITSGGSTTYADSVKGRFTISRDNAK | |
| | | NTVYLQMNSLKPEDTAVYYCNAAQS | |
| | | RTSWLFPDEYDYWGQGTQVTVSS | |
| 234 | NBX0502 | QVQLQESGGGLVQAGGSLRLSCAAS | NetB |
| | | GRTFSIYAMGWFRQAPGKEREFVAV | |
| | | INRGGGTTTYADSVKGRFTISRDNT | |
| | | KNTVSLQMNSLKPDDTAVYYCAADR | |
| | | VTDTYYYLNPESYDYWGQGTQVTVS | |
| | | S | |
| 235 | NBX0503 | QVQLQESGGGLVQAGGSLRLSCAAS | NetB |
| | | GSGRRVGYMAWYRQTPGKQRESVAT | |
| | | ISRAGATKYADSVKDRFTISRDNAK | |
| | | DTVYLQMNSLKPDDTAVYYCFASLI | |
| | | DAGTYWGQGTQVTVSS | |
| 236 | NBX0504 | QVQLQESGGGLVQAGGSLRLSCAAS | NetB |
| | | GRTFSIYAMGWFRQAPGKEREFVAV | |
| | | INRSGGTTTYADSVKGRFTISRDNT | |
| | | KNTVSLQMNSLKPDDTAVYYCAADR | |
| | | VTDTYYYLNPESYDYWGQGTQVTVS | |
| | | S | |
| 237 | NBX0505 | QVQLQESGGGLVQAGGSLRLSCAAS | NetB |
| | | GMSFSLGTIYWYRQAPGKQREFVAF | |
| | | ITNADTTMYANSVKGRFTISRDNGK | |
| | | NTVFLLMNNLKPEDSAVYYCNTATS | |
| | | WGQGTQVTVSS | |
| 238 | NBX0506 | QVQLQESGGGLVQAGGSLRVSCAAS | NetB |
| | | GSGRRVGYMAWYRQTPGKQRELVAT | |
| | | ISRAGATNYADSVKDRFTISRDNAK | |
| | | NTVYLQMNSLKPDDTAVYYCFASVF | |
| | | DAGTYWGQGTQVTVSS | |
| 239 | NBX0507 | QVQLQESGGGLVQAGGSLRLSCAAS | NetB |
| | | GSGRRVGYMAWYRQTPGKQRELVAT | |
| | | ISRAGATNYADSVKDRFTISRDNAK | |
| | | NTVYLQMNSLKPDDTAVYYCFASIF | |
| | | DAGTYWGQGTQVTVSS | |
| 240 | NBX0508 | QVQLQESGGGLVQAGGSLRLSCVAS | NetB |
| | | GSGSRINYMAWHRQTPGRQRELVAV | |
| | | INRTGAANYARSVKDRFTISRDNAK | |
| | | NTVYLQMNDLKPDDTAIYYCFASYL | |
| | | GAGAYWGQGTQVTVSS | |
| 241 | NBX0509 | QVQLQESGGGLVQAGGSLRLSCAAS | NetB |
| | | GRTFSTYTVGWFRQAPGKEREFVAS | |
| | | ITWNGGTILYADSVKGRFTISRDNA | |
| | | KNTVLLQMNSLKPEDTAVYYCVMGA | |
| | | AGQGWRYWGQGTQVTVSS | |
| 242 | NBX0510 | QVQLQESGGGLVQAGGSLRLSCVAS | NetB |
| | | GSGSRINYMAWHRQTPGRQRELVAV | |
| | | INRTGAAKYADSVKDRFTVSRDNAE | |
| | | NTVYLQMNDLKPDDTAVYYCWASYL | |
| | | GAGTYWGQGIQVTVSS | |
| 243 | NBX0511 | QVQLQESGGGLVQPGGSLRLSCAAS | NetB |
| | | GFTFSRNYMSWVRQAPGKGLEWVGS | |
| | | IYSDDSTNYAPSVKGRFTISRDNAA | |
| | | NTLYLQMNSLKSEDTAVYYCSKEGG | |
| | | LRGQGTQVTVSS | |
| 244 | NBX0512 | QVQLQQSGGGLVQAGGSLRLSCAAS | NetB |
| | | GSGRRVGYMAWYRQTPGKQRELVAT | |
| | | ISRAGATNYADSVKDRFTISRDNAK | |
| | | NTVYLQMNSLKPDDTAVYYCFASVF | |
| | | DAGTYWGQGTQVTVSS | |
| 245 | NBX0513 | QVQLQESGGGLVQAGGSLRLSCAAS | NetB |
| | | GSGRRVGYMAWYRQTPGKQRELVAT | |
| | | ISRAGATNYADSVKDRFTISRDNAK | |
| | | NTVYLQMNSLKPDDTAVYYCFASLF | |
| | | DAGTYWGQGTQVTVSS | |
| 246 | NBX0514 | QVQLQESGGGLVQAGGSLRLSCAAS | CnaA |
| | | GRTFSGRTMAWFRQAPGKEREFVAA | |
| | | ITWSGGTTYYPDSVKGRFTISRDIP | |
| | | KNTLYLQMNSLKSEDTAVYYCASDG | |
| | | PWRATTPDAYDYWGQGTQVTVSS | |
| 247 | NBX0515 | QVQLQESGGGLVQAGGSLRLSCAAS | CnaA |
| | | GSIGTIDSMGWYREAPGKRRELVAF | |
| | | IMFSGRTIYQDSVKGRFSISGDNAK | |
| | | KTVSLQMTSLKPEDTGVYYCYSNQY | |
| | | WGQGTQVTVSS | |
| 248 | NBX0517 | QVQLQQSGGGLVQPGGSLRLSCAAS | CnaA |
| | | EFSLLFGTIGWFRQAPGKEREGVSC | |
| | | VSSSDGSTYYADSVKGRFTISRDKA | |
| | | KNTWYLQMHSLKPEDTAVYYCATRC | |
| | | TVVPGITWGQGTQVTVSS | |
| 249 | NBX0518 | QVQLQESGGGVVQAGGSLRLSCVAP | CnaA |
| | | GSITRVGGMGWYRQPPGKERELVAL | |
| | | INEVGNTNYGDSVKGRFTISRDNAK | |
| | | KTVYLEMNSLKPEDTAVYYCWIPPI | |
| | | PWGQGTQVTVSS | |
| 250 | NBX0519 | QVQLQESGGGLVQPGGSLRLSCATS | CnaA |
| | | PFSLRLGVVGWFRQAPGREREGVSC | |
| | | ISSSEGSTHYADSVKGRFTISRDNA | |
| | | KNTVYLQMNSLKPEDTAVYYCATRC | |
| | | TVVPGITWGQGTQVTVSS | |
| 251 | NBX0520 | QVQLQESGGGLVQAGDSLRLSCAAS | CnaA |
| | | ARTSSSRAMGWFRQTPVREREFVAA | |
| | | ISWSGGRTAYADSVKGRFTLSKYDK | |
| | | DTVSLTMNSLKPEDTAVYYCAARRS | |
| | | DFTGDYAYSGRSAYDYWGQGTQVTV | |
| | | SS | |
| 252 | NBX0521 | QVQLQESGGGSVQAGGSLRLSCAAS | CnaA |
| | | GSTFIFDKMDWYRQTPEKSRELVAT | |
| | | LMSRGDPYYLDSVKGRFTITRDNAK | |
| | | NTVYLQMNSLKPEDTAVYVCRGRAG | |
| | | ERVYWGQGTQVTVSS | |
| 253 | NBX0522 | QVQLQESGGGLVQPGGSLRLSCAAS | CnaA |
| | | GRTFSGVIVGWFRQAPGKEREFLAT | |
| | | TLWSGGSTYYTDSVKGRFTISRDVA | |
| | | KNMVYLQMNSLKPEDAAIYYCAAKY | |
| | | GGSLSYMHPTGYTYWGQGTQVTVSS | |
| 254 | NBX0523 | QVQLQESGGGLVQAGGSLRLSCAAS | CnaA |
| | | RIVFTISTMAWFRQAPGKEREFVAS | |
| | | INRSGALTSHANSVKGRFTISRDAA | |
| | | KNTVYLQMNSLKDEDTAIYYCAASK | |
| | | ANMPALPANYDYWGQGTQVTVSS | |
| 255 | NBX0524 | QVQLQESGGGVVQAGGSLRLSCVAP | CnaA |
| | | GSITRLGSMGWYRQPPGKQRELVAL | |
| | | ITAVGNTNYGDSVKGRFTISRDNAK | |
| | | KMVYLEMNSLKPEDTAVYYCWIPPI | |
| | | PWGQGTQVTVSS | |
| 256 | NBX0525 | QVQLQESGGGVVQAGGSLRLSCVAP | CnaA |
| | | GSITRLGGMGWYRQTPGKQRELVAL | |
| | | IDTVGNTNYGESVKGRFTISRDNAK | |
| | | KMVYLEMNSLKPEDTAVYYCWIPPI | |
| | | PWGQGTQVTVSS | |
| 257 | NBX0526 | QVQLQESGGGLVQAGDSLTLSCVAS | CnaA |
| | | ERAFMYNMAWFRQAPGKERDFVAVR | |
| | | NWNVERTNYADFAKGRFTISRDAAK | |
| | | KVMYLKMNNLKPEDTAVYYCATTRV | |
| | | WPTQHQMGQIEYWGQGTQVTVSS | |
| 258 | NBX0527 | QVQLQESGGGLVQAGGSLRLSCAAS | CnaA |
| | | SSFNTMGWYRQAPGKQRELVAGITS | |
| | | GGTIKYGDSVKGRFTISGDNAKNTV | |
| | | YLQMDSLKPEDTAVYYCVADWQYGS | |
| | | TWNYWGQGTQVTVSS | |
| 259 | NBX0528 | QVQLQESGGGLVQAGDSLRLSCAAS | CnaA |
| | | GRNFDYYSMGWFRQAPGNERIFVAA | |
| | | INWRGAVIDYPDSVKGRFTISRDNA | |
| | | KNRVYLQMNSLKPEDTAVYYCAAAS | |
| | | SSSRLLEPIGYNYWGQGTQVTVSS | |
| 260 | NBX0529 | QVQLQESGGGLVQAGGSLRLSCAAS | CnaA |
| | | GSMFSINDMTWYRQAPGKQREMVAT | |
| | | ISSGGTTDYTESVKGRFFVIRDNAK | |
| | | ITVYLQMNKLRPEDSGVYYCAGNLK | |
| | | RSETSYYWKTGQGIQVTVSS | |
| 261 | NBX0530 | QVQLQESGGGLVQTGGSLKLSCATS | CnaA |
| | | GRTFSRYHMGWFRQAPGKEREFVAA | |
| | | ISLSGGGTAFANFVEGRFTISRDNA | |
| | | KNTLYLQMNSLKPEDTAVYYCTADR | |
| | | HEWGRLMKGDYWGQGTQVTVSS | |
| 262 | NBX0531 | QVQLQESGGGSVQAGGSLTVSCSAS | CnaA |
| | | GRTSNSYNMAWFRQGPGKERELVAA | |
| | | ISWTGGFTSYTNSVKDRFTISRENA | |
| | | KNTVYLQMNSLKPEDTAVYYCAATS | |
| | | RSLTSAMTREIRAYDYWGQGTQVTV | |
| | | SS | |
| 263 | NBX0532 | QVQLQESGGGLVQAGGSLRLSCAAS | CnaA |
| | | GSTFSFNKMDWYRQAPEKQRELVAT | |
| | | FMNDGNTYYVDSVKGRFTISRDNAK | |
| | | NTVYLQMNSLKFEDTAVYYCRGRAG | |
| | | MEVYWGQGTQVTVSS | |
| 264 | NBX0533 | QVQLQESGGGLVQPGGSLTLSCATS | CnaA |
| | | PLTLRLGPIGWFRQAPGKEREGVSC | |
| | | ISSRDDKNYAESVKGRFTISRDNAK | |
| | | NMVYLQMNSLKPEDTAVYYCATRCT | |
| | | VVPGISWGQGTQVTVSS | |
| 265 | NBX0534 | QVQLQESGGGLVQAGDSLRLSCAAS | CnaA |
| | | GRNFGYYTMGWFRQAPGNERIFVAA | |
| | | ITWRGVIHHADSVKGRFTISRDNAK | |
| | | NTVYLQMNSLKPEDTAVYYCAAASS | |
| | | SSRPLEPIGYNYWGQGTQVTVSS | |
| 266 | NBX0535 | QVQLQESGGGLVQAGGSLRLSCTAS | CnaA |
| | | GDIFSAAGMAWFRQTPGKERDLVAY | |
| | | VTWDGGTTRYKDSVKGRFTISRDNA | |
| | | KNTVLLQMNSLKPEDTAVYYCAAGN | |
| | | TGPFNLLHSSAQYAYWGQGTQVTVS | |
| | | S | |
| 267 | NBX0536 | QVQLQESGGGLVQAGGSLRLSCATS | CnaA |
| | | PLTLRLGAIGWFRQAPGKEREMVSC | |
| | | ITSTEDKNYADSVKGRFTISRDNAK | |
| | | TMVYLQMNSLKLEDTAVYYCATRCT | |
| | | VVPGISWGQGTQVTVSS | |
| 268 | NBX0537 | QVQLQESGGGVVQAGGSLRLSCVAP | CnaA |
| | | GSITRIGGMGWYRQPPGKQRELVAL | |
| | | INTVGNTNYGDSVKGRFTISRDNAK | |
| | | KTVYLEMNSLKPEDTAVYYCWIPPL | |
| | | PWGQGTQVTVSS | |
| 269 | NBX0538 | QVQLQQSGGGLVQAGGSLRLSCTAS | NetB |
| | | GRSFSRYIMGWFRQAPGKERESVAR | |
| | | IAPSGGSAYYADSVKGRFTISRDNA | |
| | | KNTVYLQMNNLKSEDTAVYHCAARY | |
| | | DMDYEYKTWGPGTQVTVSS | |
| 270 | NBX0539 | QVQLQESGGGLVQAGGSLRLSCVAS | NetB |
| | | GSGSRIGFMAWHRQTPGRQRELVAV | |
| | | INRTGATRYADSVKDRFTISRDNAK | |
| | | NTVYLQMNDLKPDDTALYYCFASVV | |
| | | DAGTYWGQGTQVTVSS | |
| 271 | NBX0540 | QVQLQESGGGLVQPGGSLRVSCAAS | NetB |
| | | GLTFSDYAMGWFRQAPGQEREFVAR | |
| | | ISLTAASTLYADSVRGRFTISRDNA | |
| | | KNTVYLQMNSLRPDDTAVYYCAAQG | |
| | | RILRGRGLFKASDYDYWGQGTQVTV | |
| | | SS | |
| 272 | NBX0541 | QVQLQQSGGGSVQTGGSLALSCAAS | NetB |
| | | GTISIFDPMGWYRQAPGKQRELVAS | |
| | | ISEGSTNYANSVKGRFTISRDNAKK | |
| | | TVSLQMNSLEPADTAVYYCRLSRYY | |
| | | NSNIYWGQGTQVTVSS | |
| 273 | NBX0542 | QVQLQESGGGLVQAGGSLRLSCAAS | NetB |
| | | RNIYGINVIAWYRQAPGKQREMVAR | |
| | | SANGGTTRYADSVKGRFTISRDNVK | |
| | | NIVYLQMSSLKPEDTAAYYCKAELY | |
| | | TLQHNYEYWGQGTQVTVSS | |
| 274 | NBX0543 | QVQLQESGGGSVQTGGSLALSCVAS | NetB |
| | | GTLSLFDPMGWYRQAPGKQRELVAS | |
| | | ISGLSTNYANSVKGRFTISRDDAKK | |
| | | TVSLQMNSLEPADTAVYYCHLSRYY | |
| | | NSNIYWGQGTQVTVSS | |
| 275 | NBX0544 | QVQLQESGGGLVQAGGSLRLSCAAS | NetB |
| | | GRVLSINAMGWYRQAPGKRREMVAR | |
| | | ITNGGSTNYAGSVKGRFTISRENTK | |
| | | NTMYLQMNSLKPEDTAVYYCLAEER | |
| | | PYYGGPLEYWGQGTQVTVSQ | |
| 276 | NBX0545 | QVQLQESGGGLVQAGGSLRLSCAAS | NetB |
| | | RTTFRVGTMAWFRQDPGKQRELVAG | |
| | | ITSGGSTNYADSVKGRFTISRDNAK | |
| | | NTIYLQMNSLKPEDTGIYVCFANIV | |
| | | DRPVSWGQGTQVTVSS | |
| 277 | NBX0546 | QVQLQQSGGGAVQAGGSLTLSCVAS | NetB |
| | | GSGSRIGLMAWYRQTPGRQRELVAV | |
| | | IKGTGTTRYADSVKDRFTISRDNAK | |
| | | NTMYLQMNDLKPDDTALYYCFASVL | |
| | | GAGTYWGQGTQVTVSS | |
| 278 | NBX0547 | QVQLQESGGGSVQTGGSLALSCAAS | NetB |
| | | GTISLFDSMGWYRQAPGKQRELVAS | |
| | | ITEGSTNYANSVKGRFTISRDNAKK | |
| | | TVSLQMNSLEPADTAVYYCRLSRYY | |
| | | NSNIYWGQGTQVTVSS | |
| 279 | NBX0548 | QVQLQQSGGGLVQSGGSLRLSCAAS | NetB |
| | | ETSLNFDDMRWYRQTPGKRREWVAI | |
| | | INTFPAGTTASYADSVKGRFTISKV | |
| | | NGENTVHLQMNRLKPEDTAVYYCNA | |
| | | GDYWGQGTQVTVSS | |
| 280 | NBX0549 | QVQLQESGGGLVQAGGSLRLSCTAS | NetB |
| | | GSDSSINYMGWYRQAPGKQRVLLAA | |
| | | ISRDGRSNYADSVRGRFTISRDNAK | |
| | | NTVDLQMNSLKPEDTAVYYCYVDPL | |
| | | GRVPRWGQGTQVTVSS | |
| 281 | NBX0550 | QVQLQESGGGAVQAGGSLTLSCVAS | NetB |
| | | GTVNLMAWYRQTPGRQRELVAVIKG | |
| | | TGTTRYADSVKDRFTISRDNAKNTM | |
| | | YLQMNDLKPDDTALYYCFASVLGAG | |
| | | TYWGQGTQVTVSS | |
| 282 | NBX0551 | QVQLQESGGGLVQAGGSLRLSCAAS | NetB |
| | | GSIFSRNIILWHRQAPGKQRELVGG | |
| | | INTGGRTNYESSVKGRFTISRDNAK | |
| | | NTVYLQMDRLKPEDTAVYYCNAPSL | |
| | | GYWGQGTQVTVSS | |
| 283 | NBX0552 | QVQLQQSGGGLVQAGGSLRLSCVAS | NetB |
| | | GSGSINYMAWHRQTPGRQRELVAVI | |
| | | NRTGAARYADSVKDRFTISRDNAEN | |
| | | TMYLQMNDLKPDDTAVYYCFASALG | |
| | | AGVYWGQGTQVTVSS | |
| 284 | NBX0553 | QVQLQESGGGLVQPGGSLRLSCAAS | NetB |
| | | GSGWRVGYMAWYRQTPGKQRELVAT | |
| | | ISRAGATRYEDSVKDRFTISRDNAK | |
| | | NTVYLQMNSLKPDDTAVYYCFASII | |
| | | DAGTYWGQGTPVTVSS | |
| 285 | NBX0561 | QVQLQESGGGLVQAGGSLRLSCTAS | CnaA |
| | | GENFSTYVMGWFRQAPGKEREFVAA | |
| | | HNWRGGGTYYADSVKGRFTISRDHA | |
| | | KNTVYLEMNSLKPEDTAVYYCAARS | |
| | | GGSYTYTGSYHYWGQGTQVTVSS | |
| 286 | NBX0801 | QVQLQESGGGLVQAGDSLRLSCAAA | CnaA |
| | | GRTFSSYAMGWFRQAPGKEREFVAT | |
| | | ISRSGGSTYYADSVKGRFTISRDNA | |
| | | KNTVYLQMNSLKPEDTAVYYCAANR | |
| | | YGSSSYQGQYASWGQGTQVTVSS | |
| 287 | NBX0802 | QVQLQESGGGLVQAGGSLRLSCAAS | CnaA |
| | | GRTFSSYHMGWFRQAPGKEREFVAT | |
| | | ISRSGGFTSYADSVKGRFTISRDNA | |
| | | KNTVWLQMNSLKPEDTAVYYCAAQQ | |
| | | WPDPRNPNGYDYWGQGTQVTVSS | |
| 288 | NBX0803 | QVQLQESGGGLVQAGGSLRLACAAS | CnaA |
| | | GRTFINYGMAWFRQSPGKEREFVAA | |
| | | VSISGAGTAYVEPVKDRFTISRDNT | |
| | | KNTLYLQMNTLKPEDTALYYCAAAK | |
| | | AGHWGRDANYDYWGQGTQVTVSS | |
| 289 | NBX0804 | QVQLQQSGGGLVQAGGSLRLSCSAS | CnaA |
| | | GRTLTAYGMAWFRQSPGKEREFVAA | |
| | | VSLSGASTAYVEPVKDRFTISRDNT | |
| | | QNTVYLQMNSLKPEDTALYYCAAAK | |
| | | AGQWGRDAKYDYWGQGTQVTVSS | |
| 290 | NBX0805 | QVQLQQSGGGLVQAGGSLRLSCAAS | CnaA |
| | | GRTFSTYAMGWFRQAPGKEREFVAG | |
| | | ISWSGGRISYTDSVKGRFTISRDNA | |
| | | KNTVYLQMNSLKPEDTAVYYCTADL | |
| | | KGLWALGLPGHYASWDSWGQGTQVT | |
| | | VSS | |
| 291 | NBX0806 | QVQLQESGGGLVQPGGSLRLSCAAS | CnaA |
| | | GSIGSINIMDWYRQAPGKQRDLVAT | |
| | | FTSGGSTVYADSVKGRFTISRDNAK | |
| | | DTVYLQMNSLKPEDTAVYYCRARRG | |
| | | WAIYWGQGTQVTVSS | |
| 292 | NBX0807 | QVQLQQSGGGLVQAGDSLRLSCAAS | CnaA |
| | | GRTFSSYGMGWFRQATGKEREFVAG | |
| | | ISRTGSGTAYADSVKSRFTISRDNA | |
| | | KNTVYLQMNSLKAEDTAVYYCAADS | |
| | | GGSWGRGTTYDYWGQGTQVTVSS | |
| 293 | NBX0808 | QVQLQQSGGGSVQAGGSLRLSCRAS | CnaA |
| | | ARASSIGAMAWFRQAPGKDRELVAA | |
| | | VTAGADTTYYRDFVKGRFTLSRDNA | |
| | | KNTVYLQMNSLKLDDTAVYYCAAYN | |
| | | TAGWGEPHQSYRYWGQGTQVTVSS | |
| 294 | NBX0809 | QVQLQESGGGLVQAGGSLKLSCVAS | CnaA |
| | | GLTFGNYDMAWFRQAPGKEREFVTH | |
| | | ISSSGAYTSYAYFVKGRFTISRDIA | |
| | | KNTVYLQMNSLKPEDTAIYYCAGRR | |
| | | SVVVRSFDYDYWGQGTQVTVSS | |
| 295 | NBX0810 | QVQLQQSGGGLVHPGGSLRLSCAAS | CnaA |
| | | GRIFNANGMYWYRQAPGKQRELVAS | |
| | | LYRSGSTNYLDSVKGRFTISRDNAK | |
| | | NTVYLQMNSLKPEDTAVYYCNVNWA | |
| | | LHDSWGQGTQVTVSS | |
| 296 | NBX0811 | QVQLQESGGGLVQAGDSLRLSCAAS | CnaA |
| | | ERTFSSDGMAWFRQATGKEREFVAG | |
| | | ISRTGSATAYAEFVKSRFTISRDNA | |
| | | KNTVYLQMNSLKAEDTAVYYCAANS | |
| | | GGHWWRGATYDYWGQGTQVTVSS | |
| 297 | NBX0812 | QVQLQESGGGLVQAGGSLRLSCTAS | CnaA |
| | | GTIFSANGMYWYRQALGQRRELVAS | |
| | | LYRDGSTNYADSVKGRFTISRDNAK | |
| | | NTVYLQMNSLKPEDTAVYYCNVNWA | |
| | | LHDSWGQGTQVTVSS | |
| 298 | NBX0847 | QVQLQESGGGVVQAGDSLRLSCTAS | CnaA |
| | | TRASIVGAMAWFRQAPGRNRDIVAA | |
| | | IAAGSPSTPYYADSVKGRFAISRDN | |
| | | AKNTVYLQMNSLKSEDTAIYYCAAY | |
| | | NTANWGQPHQSYRHWGQGIQVTVSS | |
| 299 | NBX0866 | QVQLQESGGGLVQPGGSLRLSAAAS | CnaA |
| | | GSILNINVMAWFRQAPGKQREWVAS | |
| | | IYRDGSTYYADSVKGRFTISRDNAK | |
| | | NTVYLQMNSLKPEDTAVYYCNVVTY | |
| | | GSNRRDFWGQGTQVTVST | |
| 300 | NBX0867 | QVQLQESGGGLVQAGDSLRLSCAAS | CnaA |
| | | GRTFSSYAMGWFRQAPGKDREFVST | |
| | | ISRSGGSTYYADSVKGRFTISRDNA | |
| | | KNTVYLQMNSLKPEDTAVYYCAANR | |
| | | YGSSSYQGQYGSWGQGTQVTVSS | |
| 301 | NBX0868 | QVQLQQSGGGLVQAGDSLRLSCAAS | CnaA |
| | | GRTFSSYAMGWFRQAPGKEREFVAS | |
| | | ISRSGGSTYYADSVKVRFTISRDNA | |
| | | KNTVYLQMNSLKPEDTAVYYCAANR | |
| | | YGSSSYQGQYDYWGQGTQVTVSS | |
| 302 | NBX0869 | QVQLQESGGGLVQAGGSLRLSCTAS | CnaA |
| | | GTIFSINGMYWYRQALGKRRELVAS | |
| | | LYRGGSTNYADSVKGRFTISRDNAK | |
| | | NTVYLQMNSLKPEDTAVYYCNVNWA | |
| | | LQDSWGQGTQVTVSS | |
| 303 | NBX0870 | QVQLQESGGGLVQAGGSLRLSCAAS | CnaA |
| | | TSDGSINVMDWYRQTPGKQRDLVAT | |
| | | ITSLGSQVYADSVKGRFTISRDNAK | |
| | | DTVYLQMNSLKPEDTAVYYCRARRG | |
| | | WAIYWGQGTQVTVSS | |
| 304 | NBX0871 | QVQLQQSGGGLVQAGGSLRLSCAAS | CnaA |
| | | GRTFNIYAMGWFRQAPGKEREFVAG | |
| | | ISDSGGSANYADSVKDRFTISMDNA | |
| | | KNTVYLQMNSLKPEDTAVYYCAADL | |
| | | TGLWALGLPGHYASWDSWGQGTQVT | |
| | | VSS | |
| 305 | NBX0872 | QVQLQESGGGLVQPGGSLRLSCAAS | CnaA |
| | | GFTFRSSAMSWVRQVPGKGLEWVSS | |
| | | IGSDGENIYYADAVKGRFTISRDNA | |
| | | KNTMYLQMNSLKLEDTAVYYCOLGR | |
| | | TVLDYFKGQGTQVTVSS | |
| 306 | NBX0873 | QVQLQESGGGLVQPGGSLRLSCAAS | CnaA |
| | | GRTFINYGMAWFRQSPGKEREFVAA | |
| | | VSSSGAGTAYVEPVKDRFTISRNNT | |
| | | KNTVYLQMNSLKPEDTALYYCAAAK | |
| | | AGQWGRYANYDYWGQGTQVTVSS | |
| 307 | NBX0874 | QVQLQQSGGGLVQAGDSLRLSCAAS | CnaA |
| | | GRTFSSYAMGWFRQAPGKEREFVAA | |
| | | ISRSGGTTYYADSVKGRFTISRDNA | |
| | | KNTVYLQMNTLKPEDTAVYYCAANP | |
| | | YGSSSYQGQYGSWGQGTQVTVSS | |
| 308 | NBX0875 | QVQLQQSGGGLVQAGDSLRLSCAAS | CnaA |
| | | GRAFSGYAMGWFRQAPGREREFVAA | |
| | | ISRGGGTTYYADSVKGRFTISRDNA | |
| | | KNTVYLQMNSLKPEDTAVYYCAANR | |
| | | YGSSSYQGQYGSWGQGTQVTVSS | |
| 309 | NBX0876 | QVQLQESGGGLVQAGGSLRLSCAAS | CnaA |
| | | GRTFINYGMAWFRQSPGKEREFVAA | |
| | | VSSSGAGTAYVEPVKDRFTISRDNT | |
| | | KNTVYLQMDTLKPEDTALYYCAAAK | |
| | | AGHWGRDANYDYWGQGTQVTVSS | |
| 310 | NBX0877 | QVQLQESGGGMVEPGGSLRLSCAAS | NetB |
| | | GSISSITFMGWHRQAPGKEGEFVAL | |
| | | IARSGTTTYADSVKGRFSISRDNAK | |
| | | NTVYLQMNNLKPEDTALYYCYVDRR | |
| | | GAVPTWGQGTQVTVSS | |
| 311 | NBX0878 | QVQLQQSGGGLVEPGGSLRLSCAAS | NetB |
| | | GSISSITFMGWHRQAPGEQGELVAL | |
| | | IARSGTTTYADSVKGRFTISRDNAK | |
| | | NTVYLQMNNLKPEDTALYYCYVDRR | |
| | | DVVPTWGQGTQVTVSS | |
| 312 | NBX0879 | QVQLQESGGGLVQAGGSLRLSCAAS | NetB |
| | | GTGFPIITFMGYYRQAPGNQRELVA | |
| | | IISRGGVAKYGDSVKDRFTISRDNA | |
| | | KNTVYLEMNSLKPDDTAVYYCYADR | |
| | | ESGSPTWGQGTQVTVSS | |
| 313 | NBX0880 | QVQLQESGGGLVQPGGSLRLSCAAS | NetB |
| | | VSSIGTMGWFRQAPGKQPELVASIS | |
| | | RVGTTNYANSVKGRFTVSRDNAQNT | |
| | | MYLQMNSLKPEDTAVYLCFANVISG | |
| | | PVYWGQGTQVTVSS | |
| 314 | NBX0881 | QVQLQESGGGLVQAGGSLRLSCAAS | NetB |
| | | TRFFSNYAMGWFRQAPGKEREFVAA | |
| | | ISRDGAVPLSGNSVPGRFTISRDNA | |
| | | KNTLYLQMNSLKPEDSAVYYCAASR | |
| | | QGNPYAQTSYDYWGQGTQVTVSS | |
| 315 | NBX0883 | QVQLQESGGEVVAPGGSLSLSCVAS | NetB |
| | | GSADSIKIMGWYRQAPGKQRELVAT | |
| | | ITSGGTTEFAESVKGRFTISRDNAK | |
| | | NTLYMQMNSLSPEDTAVYYCNALVS | |
| | | RRDSAAYFAWGQGTQVTVSS | |
| 316 | NBX0884 | QVQLQESGGGLVQPGGSLRLSCAAS | NetB |
| | | ESIVSITPMMWYRQAPGKQREWVAI | |
| | | TTRDGAPAYADSVKGRFTISRDSAK | |
| | | NTVYLQMNYLKPEDTAVYFCKARKD | |
| | | SHDYWGQGTQVTVSS | |
| 317 | NBX0885 | QVQLQESGGGLVQAGGSLRLSCAAS | NetB |
| | | ETIGSIQRMGWYRQAPGKQRELVAT | |
| | | RTNGGTTNYGDSVRGRFTISVDVAK | |
| | | NTVYLQMNSLKPEDTAVYYCNAHIR | |
| | | EYYSTYDYWGQGTQVTVSS | |
| 318 | NBX0886 | QVQLQESGGGLVQPGGSLRLSCSAS | NetB |
| | | GSISRIRDMAWHRQVPGKQRELVAS | |
| | | ISSGGSTNVADSLKGRFTISRDNGK | |
| | | NTMYLQMDSLKSEDTAVYYCNALFN | |
| | | PIDGPARYYWGQGTQVTVSS | |
| 319 | NBX0887 | QVQLQESGGGLVQPGGSLRLSCSAS | NetB |
| | | GSISRIYDMAWHRQVPGKQRELVAG | |
| | | ISRGGSTNVADSLKGRFTISRDNGK | |
| | | NTVYLQMDNLKSEDTAVYYCTALFN | |
| | | PVDGTARYYWGQGTQVTVSS | |
| 320 | NBX0888 | QVQLQESGGGLVQAGGSLRLSCAAS | NetB |
| | | GTIFSINVMGWYRQAPGKQRELVAS | |
| | | ITSGGQIKYADSVKGRFTTSRDNAK | |
| | | NTVYLQMNSLKPEDTAVYYCNAASS | |
| | | TWPPRDYDYWGQGTQVTVSS | |
| 321 | NBX0889 | QVQLQESGGGLVQPGGSLRLSCAAS | NetB |
| | | RSISSIAAMGWYRQAPGKQRELVAR | |
| | | ITNGGSTNYADSVKGRFTISRDNAK | |
| | | NTVYLQMNSLKPEDTAVYYCNADER | |
| | | PYYGDSVLSWGQGTQVTVSS | |
| 322 | NBX0890 | QVQLQQSGGGLVQAGGSLRLSCAAS | NetB |
| | | GTGFPIITFMGYYRQTPGNQREEVA | |
| | | LINRGGVAKYGDSVKDRFTISRDNA | |
| | | KNTVYLEMNNLKPDDTAVYYCYADR | |
| | | ESGSPTWGQGTQVTVSS | |
| 323 | NBX0891 | QVQLQESGGGLVQAGGSLRLSCAAS | NetB |
| | | GRTFSNYHMAWFRQAPGKEREFVAA | |
| | | ISRGTSTTFYRDSVRDRFTISRDNA | |
| | | KNTAYLQMNSLKPEDTAVYYCAADA | |
| | | DRSTTIYSRDIYDYWGQGTQVTVSS | |
| 324 | NBX0892 | QVQLQESGGGLVQAGDSLRLSCAAS | NetB |
| | | EGTFSNYRMGWFRQAPGKEREFVAA | |
| | | ISRDGAVPLSGNSPLGRFTISRDNA | |
| | | KNTLYLQMNSLKPEDTAVYYCAASR | |
| | | QGLPYVETSYDYWGQGTQVTVSS | |
| 325 | NBX0893 | QVQLQESGGGLVQPGGSLRLSCVAS | NetB |
| | | GSISSITFMGWYRQVLGEQRELVAL | |
| | | SARRGTTTYADSVKGRFTISRDNAK | |
| | | NTVYLQMNNLKPEDTALYYCYVDRR | |
| | | DEVPTWGQGTQVTVSS | |
| 326 | NBX0894 | QVQLQQSGGGLVQAGGSLRLSCAAS | NetB |
| | | GGTFSSYVMAWFRQAPGKEREFLAA | |
| | | IRWSRGSTYYADSVKGRFTVFRDTV | |
| | | ENTVYLQMNSLKPEDTAVYYCAADG | |
| | | NPAKLVLDQYGMDYWGKGTLVTVSS | |
| 327 | NBX0895 | QVQLQQSGGGLVEPGGSLRLSCAAS | NetB |
| | | GSISEITYMGWHRQAPGEQRELVAL | |
| | | IARVGTTRYADSVKGRFTISRDNAK | |
| | | NTVYLQMNNLKPEDTALYYCYVDQR | |
| | | GVVPTWGQGTQVTVSS | |
| 328 | NBX0896 | QVQLQESGGGSVQAGGSLRLSCRAS | CnaA |
| | | ARASSIGAMAWFRQAPGKDRELVAA | |
| | | VNAGADTTYYRDFVKGRFTISRDNA | |
| | | KNTVYLQMNSLKLDDTAVYYCAAYN | |
| | | TAGWGEPHQSYRYWGQGIQVTVSS | |
| 329 | NBX0897 | QVQLQESGGGLVQPGGSLSLSCAAS | CnaA |
| | | GSIFIISTMGWYRQAPGKQRELVAT | |
| | | ITSGGSTNYADPVKGRFTISRDNAK | |
| | | NMVYLQMNSLKPEDTAVYYCNAEVH | |
| | | VWGVPGPRDYWGQGTQVTVSS | |
| 330 | NBX0898 | QVQLQESGGGLVQAGDSLRLSCAAS | CnaA |
| | | GRTFSSYAMGWFRQAPGKEREFVAT | |
| | | ISRSGGSTYYADSVKGRFTISRDNA | |
| | | KNTVYLQMNSLKPEDTAVYYCAANP | |
| | | YGSSSYQGQYASWGQGTQVTVSS | |
| 331 | NBX0899 | QVQLQQSGGGLVQAGGSLRLSCAAS | CnaA |
| | | GSIFSSNGMYWYRQAPGKQRELVAS | |
| | | LYRSGSTNYADSVKGRFIISRDNAK | |
| | | NTVYLQMNSLKPEDTAVYYCNVNWA | |
| | | LHDSWGQGTQVTVSS | |
| 332 | NBX08100 | QVQLQESGGGLVQAGGSLRLSCAAS | CnaA |
| | | GRTFSAYGMAWFRQSPGKEREFVAA | |
| | | VSGGGGGTAYAEPVKDRFTISRDNA | |
| | | KNTVYLQMNTLKPEDTALYYCAAAT | |
| | | AGHWGRDANYDYWGQGTQVTVSS | |
| 333 | NBX08101 | QVQLQESGGGLVQAGGSLRLSCAAS | CnaA |
| | | GSIFSSNGMYWYRQAPGKQRELVAS | |
| | | LFRSGSTNYADSVKGRFTISRDNAQ | |
| | | NTVYLRMNSLKPEDTAVYYCNVNWA | |
| | | LHDSWGPGTQVTVSS | |
| 334 | NBX08102 | QVQLQESGGGLVQAGDSLRLSCAAS | CnaA |
| | | GRTFSSYAMGWFRQAPGKEREFVAA | |
| | | ISRSGGTTYYADSVKGRFTISRDNA | |
| | | KNTVYLQMNTLKPEDTAVYYCAANP | |
| | | YGSSSYQGQYGSWGQGTQVTVSS | |
| 335 | NBX08103 | QVQLQESGGGLVQPGGSLRLSCAAS | CnaA |
| | | GIIHSINVMGWYRQAPGKQRELVAI | |
| | | ISSGGRTTYADSVKGRSTITGDNDK | |
| | | NTVYLQMNSLKPEDTAVYYCTMVWG | |
| | | LRYYWGQGTQVTVSS | |
| 336 | NBX08104 | QVQLQQSGGGFVRPGESLTLSCAAS | CnaA |
| | | TSIFSSNGMYWYRQAPGKRRELVAS | |
| | | LFRSGSTNYADSVKGRFTISRDNAK | |
| | | NTVYLQMNSLKPEDTAVYYCNVNWA | |
| | | LHDSWGQGTQVTVSS | |
| 337 | NBX08105 | QVQLQESGGGLVQAGDSLRLSCAAS | CnaA |
| | | GRTFSSYAMAWFRQAPGKEREFVAA | |
| | | ISRGGGTTYYADSVKGRFTISRDNA | |
| | | KNTVYLQMNSLKPEDTAVYYCAANP | |
| | | YGSSSYQGQYGSWGQGTQVTVSS | |
| 338 | NBX08106 | QVQLQESGGGLVQAGGSLRLSCAAS | CnaA |
| | | GSIFSSNGMYWYRQAPGKQRELVAS | |
| | | LYRSGSTNYADSVKGRFIISRDNAK | |
| | | NTVYLQMNSLKPEDTAVYYCNVNWA | |
| | | LHDSWGQGTQVTVSS | |
| 339 | NBX08107 | QVQLQQSGGGEVQPGGSLRLSCAAS | CnaA |
| | | GSIFSSNGMYWYRQAPGKQRELVAS | |
| | | LYRSGSTNYADSVKGRFIISRDNAK | |
| | | NTVYLQMNSLKPEDTAVYYCNVNWA | |
| | | LHDSWGQGTQVTVSS | |
| 340 | NBX08108 | QVQLQESGGGLVQAGGSLRLSCAAS | CnaA |
| | | RSILSANGMYWYRQAPGKQRELVAS | |
| | | LYRSGSTDYADSVKGRFTISRDDSR | |
| | | DTMYLQMNSLKPEDTAVYYCNVNWA | |
| | | LHDSWGQGTQVTVSS | |
| 695 | NBX03161 | QVQLQESGGGLVQAGGSLRLSCAAS | Cpa |
| | | GRSLSTVSMGWFRQAPGKEREFVAA | |
| | | IAWSGGRTTYADSVKGRFTISRDNA | |
| | | TNTVYLQMNSLKPEDTAVYYCAGYR | |
| | | GVLFITTKSAYDYWGQGTQVTVSS | |
| 696 | NBX03162 | QVQLQESGGGLVQPGGSLRLSCAAS | Cpa |
| | | GFTFSSYWMYWVRQAPGKGLEWVSS | |
| | | ISTGGTSTYYTDSVKGRFTISRDNA | |
| | | KNTLYLQMNSLKPEDTALYYCAKLV | |
| | | AYGMDYWGKGTLVTVSS | |
| 697 | NBX03163 | QVQLQESGGGLVQAGDSLRLSCATS | Cpa |
| | | GRTFSRYTMGWFRQTPGKEREFVAA | |
| | | ISWSGTYYTDSVKGRFTISRDNAKN | |
| | | TVYLQMNSLKPEDTAVYYCAVGSRR | |
| | | LYYSSDINYWGQGTQVTVSS | |
| 698 | NBX03164 | QVQLQQSGGGLVQAGDSLRLSCATS | Cpa |
| | | GRTFSSYTMGWFRQTPGKEREFVAA | |
| | | ISWSGTYYTDSVKGRFTISRDNAKN | |
| | | TVYLQMNSLKPEDTAVYYCVVGSRR | |
| | | LYYSSDINYWGQGTQVTVSS | |
| 699 | NBX03165 | QVQLQESGGGLVQAGDSLRLSCATS | Cpa |
| | | ERTFSRYTMGWFRQTPGKEREFVAA | |
| | | ISWSGTYYTDSVKGRFTISTDNAKR | |
| | | TVYLQMNNLKPEDTAVYYCAQGSRR | |
| | | LYYSSDIDYWGQGTQVTVSS | |
| 700 | NBX03166 | QVQLQESGGGLVQAGGSLRLSCTAS | Cpa |
| | | GFIVNSYWMHWFRQAPGKGLEWVAA | |
| | | ISTNAYSTYYADSVRGRFTTSRANA | |
| | | ENTPYLEMNNLKPEDAALYYCARGG | |
| | | TAQEGGMDYWGQGTLITVSS | |
| 701 | NBX03167 | QVQLQESGGGLVQAGDSLRLSCATS | Cpa |
| | | GHDFSKYTMGWFRQTPGKEREFVAA | |
| | | ISWSGTYYTDSVKGRFTISRDNAKN | |
| | | TVYLQMNSLKPEDTAVYYCAVGSRR | |
| | | LYYSSDIDYWGQGTQVTVSS | |
| 702 | NBX03168 | QVQLQQSGGGLVQAGDSLRLSCATS | Cpa |
| | | GFTFSRYTMGWFRQTPGKEREFVAA | |
| | | ISWSGTYYTDSVKGRFTISRDNAKN | |
| | | TVYLQMNSLKPEDTAVYYCASGSRR | |
| | | LYYSSDIDYWGQGTQVTVSS | |
| 703 | NBX03169 | QVQLQESGGGLVQAGDSLRLSCATS | Cpa |
| | | GRTFSRYTMGWYRQTPGKEREFVAA | |
| | | ISWSGTYYTDSVKGRFTISIDNAKN | |
| | | TVYLQMNSLKPEDTAVYYCASGSRR | |
| | | LYYSSDIDYWGQGTQVTVSS | |
| 704 | NBX03170 | QVQLQQSGGGLVQAGDSLRLSCATS | Cpa |
| | | GRTFSRYTMGWFRQSPGKEREFVAA | |
| | | ISWSGTYYTDSVKGRFTISRDNANN | |
| | | TVYLQMNSLKPEDTAVYYCVVGSRR | |
| | | LYYSSDIDYWGQGTQVTVSS | |
| 705 | NBX03171 | QVQLQQSGGGLVQAGDSLRLSCATS | Cpa |
| | | GRDFSRYTMGWFRQTPGKEREFVAA | |
| | | ISWSGTYYTDSVNGRFTISRDNAKN | |
| | | TVYLQMNSLKPEDTAVYYCAVGSRR | |
| | | LYYSSDIDYWGQGTQVTVSS | |
| 706 | NBX03172 | QVQLQESGGGLVQAGDSRRLSCATS | Cpa |
| | | GGTFSSYTMGWFRQTPGKEREFVAA | |
| | | ISWSGTYYADSVKGRFTISRDNAKN | |
| | | TVYLQMNSLKPEDTAVYYCAVGSRR | |
| | | LYYSSDINYWGQGTQVTVSS | |
| 707 | NBX03173 | QVQLQESGGGLVQAGGSLRLSCAAS | Cpa |
| | | GRTFSSYAMGWFRQAPGKEREFVGA | |
| | | LSGSGATTDYADFVKSRFTISRDNA | |
| | | KNTVYLQMRDLKAEDTAVYYCAASG | |
| | | HFGLRGTYNYEYWGQGTQVTVSS | |
| 708 | NBX03174 | QVQLQESGGELVQAGDSLRLSCAVS | Cpa |
| | | GRTGIKWTMAWFRQAPGKEREFVAR | |
| | | ITWSGDTVYADSMKGRFTISRDNAK | |
| | | NTMYLQMNRLKPEDTAVYTCAADRI | |
| | | YNEDRYYYWGQGTQVTVSS | |
| 709 | NBX03175 | QVQLQESGGGLVQAGGSLRLSCAAS | Cpa |
| | | GSIFRIIAMAWYRQAPGKQRELVAA | |
| | | ITNGGATMYADAVKGRFTISRDTAK | |
| | | NTAYLQMNSLKLEDTAVYYCAADHF | |
| | | GRTPYWGQGTQVTVSS | |
| 710 | NBX03176 | QVQLQESGGGLVQAGDSLRLSCATS | Cpa |
| | | GRTFSSYTMGWFRQTPGKEREFVAA | |
| | | ISWSGTYYTDSVKGRFTISRDNAKN | |
| | | TVYLQMNSLKPADTAVYYCAVGSRR | |
| | | LYYSSDIDYWGQGTQVTVSS | |
| 711 | NBX03177 | QVQLQQSGGGLVQAGGSLRLSCAAS | Cpa |
| | | SRTFSNYAMAWFRQTPGKEREFLAT | |
| | | ISGVTAFTIYADSVKGRFTISRDNA | |
| | | KNTLYLQMNSLKAEDTAVYYCAARQ | |
| | | WNPTMRERDYDYWGQGTEVTVSS | |
| 712 | NBX03179 | QVQLQESGGGLVQTGDSLRLSCATS | Cpa |
| | | GRSFSRYTMGWFRQTPGKEREFVAA | |
| | | ISWSGTYYTDSVKGRFTISRDNAKN | |
| | | TVYLQMNSLKPEDTAIYYCAVGTRR | |
| | | LYYSSDIDYWGQGTQVIVSS | |
| 713 | NBX03180 | QVQLQQSGGGLVQAGDSLRLSCATS | Cpa |
| | | GLTVSRYTMGWFRQTPGKEREFVAT | |
| | | ISWSGTYYTDSVKGRFTISRDDAKN | |
| | | MIYLQMNSLKPEDTAVYYCAAGSRR | |
| | | LHYSSDIDYWGQGTQVTVSS | |
| 714 | NBX03181 | QVQLQESGGGLVQAGDSLRLSCATS | Cpa |
| | | GLTFSRYTMGWFRQTPGKEREFVAA | |
| | | ISWSGTYYSDSVKGRFTISRDNAKN | |
| | | THYLQMNSLKPEDTAVYYCVVGSRR | |
| | | LYYSSDINYWGQGTQVTVSS | |
| 715 | NBX0570 | QVQLQESGGGLVQAGGSLRLSCAAS | NetB |
| | | GTGRRFGYMAWYRQPPGKEREMVAT | |
| | | ISRAGATNYADSVKDRFTISRDNIK | |
| | | NTVYLQMNSLKPDDTAVYYCFASVF | |
| | | DAGTYWGQGTQVTVSS | |
| 716 | NBX0571 | QVQLQESGGGLVQAGGSLRLSCAAS | NetB |
| | | GSIFSINVMGWYRQAPGKQRELVAD | |
| | | ATSGGMTNYADSVKGRFTISRDNAK | |
| | | NTVYLQMNSLKPEDTAVYYCNAAQS | |
| | | RTSRLFPDEYDYWGQGTQVTVSS | |
| 717 | NBX0578 | QVQLQESGGGLVQAGGSLRLSCTAS | CnaA |
| | | GRDFSTYVMGWFRQAPGKEREFVAA | |
| | | SNWNDGGTYYADSVKGRFTISRDNA | |
| | | KNTVYLQMNSLKPEDTAVYFCAARS | |
| | | GGSYTYTGSYHYWGQGTQVTVSS | |
| 718 | NBX0579 | QVQLQESGGGLVQAGGPLRLSCAAS | CnaA |
| | | GRTLYNNAMGWFRQAPGKEREFVAG | |
| | | ISSSGISTDYADSVKGRFTISRDNA | |
| | | KNTVYLQMKSLKSADTALYYCAASR | |
| | | AATVGVTPQEYAYWGPGTQVTVSS | |
| 719 | NBX0580 | QVQLQESGGGLVQPGATLRLSCAVS | CnaA |
| | | GRTISNYGVGWFRQAPGKEREFVGV | |
| | | SWSASSTVYADSMKGRFAISRDNVK | |
| | | NTVSLQMNSLRPEDTAIYYCAMMGA | |
| | | SYCAGYRCHHAAQTYDYWGQGTQVT | |
| | | VSS | |
| 720 | NBX0581 | QVQLQESGGGLVQAGGSLTLSCAAS | CnaA |
| | | GPPSRRYDMGWFRQAPGKEREFVAA | |
| | | VSWTGSSTYYSDSVKGRFTISRDGV | |
| | | KNTVYLQMNSLKSEDTAVYYCAVKN | |
| | | TYGSSSYYYTSSSYDYWGQGTQVTV | |
| | | SS | |
| 721 | NBX0582 | QVQLQESGGGLVQAGGSLRLSCTVA | CnaA |
| | | GEIDSAAGMAWFRQAPGKERELVAA | |
| | | VTWDGGTTRYKDSVKGRFTISRDHA | |
| | | KNTVLLQMNSLKPEDTAVYYCAAGN | |
| | | TGPFNLLYSSAQYLYWGQGTQVTVS | |
| | | S | |
| 722 | NBX0584 | QVQLQESGGGLVQAGGSLRLSCAAS | CnaA |
| | | GSMFSIHDMSWVRQAPGKQREVVAT | |
| | | VSSDGTTNYAESVKGRFTVSRDNAK | |
| | | ITAYLQMNSLKLEDTGVYYCMGNLR | |
| | | RLDSGYADSYYYKTGQGIQVTVSS | |
| 723 | NBX0585 | QVQLQQSGGGLVQAGGSLRLSCAAS | CnaA |
| | | GRTADRYAISWFRQAPGEERDFVAR | |
| | | IERSGGATVYANSVKGRFTISRDNA | |
| | | KNLVYLQMNSLKPEDTAVYYCAARL | |
| | | ALAGEYDYWGQGTQVTVSS | |
| 724 | NBX0586 | QVQLQESGPGLVKPSQTLSLTCTVS | Cpa |
| | | GGSIRTDRYYWSWIRQPPGKGLEWM | |
| | | GAIAWTASTFYNPSLKSRTSISRDT | |
| | | SGNQFTLQLSSVTPEDTAVYYCVPI | |
| | | VGDSSWSGGSWGQGTQVTVSS | |
| 725 | NBX0587 | QVQLQESGGGLVQAGGSLRLSCAVS | Cpa |
| | | GIIFSINAMGWYRQAPGKQREYVAG | |
| | | ITGRGRTNYVDSVKGRFTISRDNAR | |
| | | NTVYLQMNSLKPEDTGVYYCNEARN | |
| | | GLGSPANSWGQGTQVTVSS | |
| 726 | NBX08116 | QVQLQESGGGLVQVGGSLRLSCAAS | NetB |
| | | GSIFSIYTMGWYRQAPGNEREMVAT | |
| | | ITSGGTTNYADSVQGRFTITTSRDN | |
| | | AKNTVYLQMNSLKPEDTAVYSCNAL | |
| | | VNQRDGLATYYWGQGTQVTVSS | |
| 727 | NBX08117 | QVQLQESGGGLVQAGGSLRLSCAAS | NetB |
| | | GSISSISGMGWYRQAPGRQRALVAT | |
| | | ITSYGSTDYEDSVKGRFTISRDNAK | |
| | | NTVYLQMNSLKPEDTGVYYCHAVQG | |
| | | STWWGSGSWGQGTQVTVSS | |
| 728 | NBX08118 | QVQLQQSGGGLVQAGGSHRLSCAAS | NetB |
| | | KSIGSIHRMGWYRQAPGKERELVAI | |
| | | ITNGGSTNHRDSVRGRFTISADVAK | |
| | | NTVYLQMNSLKPEDTAVYYCYANIR | |
| | | EYYSSYEYWGQGTQVTVSS | |
| 729 | NBX08119 | QVQLQESGGGLVRAGDSLTLSCAAS | NetB |
| | | GRTFSSYAMGWFRQAPGKEREFVAA | |
| | | INRSGGTTSYVEPVKGRFTISRDNA | |
| | | KNTVYLQMNSLKPEDTAVYYCAADR | |
| | | VGDDYYYISSQHYDYWGQGTQVTVS | |
| | | S | |
| 730 | NBX08120 | QVQLQESGGGLVQAGGSLRLSCTIS | NetB |
| | | GTRGSINPVAWSRQAPGKQREPIAF | |
| | | ISTGGATIYGDSAKGRFTISRDNAK | |
| | | NTVYLQMSNLKPEDSGVYYCNTESY | |
| | | RGQGTQVTVSS | |
| 731 | NBX08121 | QVQLQQSGGGLVQAGDSLRLSCTAS | NetB |
| | | GRTFNGKGMAWFRQAPGKEREFVAG | |
| | | INWSGDSTSYGDSVKGRFTISRDDA | |
| | | KNTVYLQMNSLKLEDTAVYYCAVST | |
| | | YSTYWFTPARYDYWGQGTQVTVSS | |
| 732 | NBX08122 | QVQLQESGGGLVQAGGSLRLSCLAS | NetB |
| | | GMTFSDHAMGWYRQAPGKQRELVAI | |
| | | IGRGGTTTYFDSVKGRFTISKDNAK | |
| | | NTLYLQMNSLKPEDTAVYYCNVVPL | |
| | | RAGIDTYWGQGTQVTVSS | |
| 733 | NBX08123 | QVQLQESGGGLVQAGGSLRLSCAAS | NetB |
| | | GSGRRVGYMAWYRQTPGKQRELVAT | |
| | | ISRAGATKYEDSVKDRFTISRDNAK | |
| | | NTVYLQMNSLKPDDTAVYYCFASII | |
| | | DAGTYWGQGTQVTVSS | |
| 734 | NBX08124 | QVQLQESGGGLVEPGGSLRLSCAAS | NetB |
| | | GIISSITYMGWYRQAPGKQRELVAL | |
| | | IARSGATRYADSVKDRFTISRDDAK | |
| | | NTVYLQMNNLKAEDTALYYCYIDQR | |
| | | DVVPTWGQGTQVTVSS | |
| 735 | NBX08125 | QVQLQESGGGLVQPGGSLRLSCAAS | NetB |
| | | QSIFRINAMGWYRHTPGKPRELVAL | |
| | | ITSGGVTTYADYVKGRFTISGDNAK | |
| | | NTVYLQMNSLKPEDTAVYECHARSW | |
| | | SADYWGQGTQVTVSS | |
| 736 | NBX08126 | QVQLQQSGGGLVQTGGSLRLSCAAS | NetB |
| | | GIMLRDEALGWYRQAPGRQREMVAI | |
| | | ITRGGATNYFGSVKGRFTISRDNAK | |
| | | NTVYLQMNSLKPEDTAVYFCRIIGT | |
| | | DTWGQGTQVTVSS | |
| 737 | NBX08127 | QVQLQESGGGLVQPGGSLRLSCAAS | NetB |
| | | GDTISSYTMAWFRQAPGKDREFVAS | |
| | | ITWSGEVTYYADSVKGRFTISRDNA | |
| | | KNTLYLQMNSLKPEDTAVYYCAAGR | |
| | | AGTNWNYWGQGTQVTVSS | |
| 738 | NBX08128 | QVQLQESGGGLVQAGGSLRLSCAAS | NetB |
| | | RIISSITDMGWYRQPPGQRELVASI | |
| | | SRGGRIDYADSVKGRFSISRDNAQN | |
| | | AVYLQMNSLKPEDTDVYYCSALSSG | |
| | | KYYWGQGTQVTVSS | |
| 739 | NBX08129 | QVQLQESGGGPVQAGGSLRLSCAAS | NetB |
| | | APLNSIHAMRWFRQVPGKQREMVAT | |
| | | STNGGSTNYHESVKGRFTISRDNAK | |
| | | NAVYLEMNSLKPEDSAVYYCYAEVR | |
| | | HYSGNVDQYWGQGTQVTVSS | |
| 740 | NBX08138 | QVQLQQSGGGLVQAGGSLRLSCAAS | CnaA |
| | | GRIFGINGWYWYRQAPGKRRELVAS | |
| | | LYRSGGTNYLDSVKGRFIISRDNAK | |
| | | NTVYLQMNDLKPEDTAVYYCNVNWA | |
| | | ESDSWGQGTQVTVSS | |
| 741 | NBX08139 | QVQLQESGGGSVQAGGSLRLSCAAS | CnaA |
| | | RGTFSNLGLAWFRQAPGKEREFVAA | |
| | | LKRDGNRTYYADSMKGRFTISRDNA | |
| | | KNTVYLQMNSLKPEDTAVYYCAARL | |
| | | PRPNAVVDTTSEYDYWGQGTQVTVS | |
| | | S | |
| 742 | NBX08140 | QVQLQESGGGLVQAGGSVRLSCVAS | CnaA |
| | | GRTFNMAWFRQGPGNEREFVASISG | |
| | | SGGITHVADSVKGRFTISRDTDKNT | |
| | | LYLHMDSLKPEDTAVYYCAVARFGG | |
| | | AWVYGGQGTQVTVSS | |
| 743 | NBX08141 | QVQLQQSGGGLVQPGGSLRLSCAAS | CnaA |
| | | GRTFSDLGMGWFRQAPGKEREFVAQ | |
| | | ILSGTTRPVYGDSVEGRFTISTDSA | |
| | | KNTVYLRMNRLEPEDTAVYYCAART | |
| | | VGGLPIWGPGTQVTVSS | |
| 744 | NBX08142 | QVQLQESGGGLVQAGGSLRLSCTAS | CnaA |
| | | DTIFSSNGMYWYRQALGKRRELVAS | |
| | | LYRDGNTAYADSVKGRFTISRDNAK | |
| | | NMVWLQMNSLKPEDTAVYYCNINWP | |
| | | LHDSWGQGTQVTVSS | |
| 745 | NBX08143 | QVQLQESGGGSVQAGDSLRLSCAVS | CnaA |
| | | RGTFNNYGLAWFRQAPGKEREFVAS | |
| | | LTRLGGRTYYADSVKGRFTISRDNA | |
| | | KNTVYLQMNSLKPEDTAVYYCAARL | |
| | | PRTNAVVDDISEYTYWGHGTQVTVS | |
| | | S | |
| 746 | NBX08144 | QVQLQQSGGGSVQAGGSLRLSCAAS | CnaA |
| | | RGTFNNLGMAWFRQAPGKEREFVAA | |
| | | LKRDGIRTYYGDSMKGRCTISRDNA | |
| | | KITVYLQMNSLKSEDTAVYYCAARL | |
| | | PRTNAVVDSTSEYDYWGQGTQVTVS | |
| | | S | |
| 747 | NBX08145 | QVQLQQSGGGLVQAGGSLRLSCAAS | CnaA |
| | | GRIFGINGWYWYRQAPGKRRELVVS | |
| | | LYRSGGTNYLDSVKGRFIISRDNAK | |
| | | NTVYLQMNDLKPEDTAVYYCNVNWA | |
| | | ESDSWGQGTQVTVSS | |
| 748 | NBX08149 | QVQLQESGGGLVQAGGSLRLSCVAS | NetB |
| | | GSPLSINVMGWYRQTPGKQRELVAS | |
| | | ITSGGQTRYAESVKGRFTISRDGGK | |
| | | NTVVLQMNSLNFEDAAVYYCNARDA | |
| | | TYYVEYNYWGQGTQVTVSS | |
| 749 | NBX08150 | QVQLQESGGGLVQAGGSLRLSCAAS | NetB |
| | | ARTFSDYAMGWFRQPPGEGREFVAG | |
| | | IRENGGRTEYADSVKGRFTVSRDNV | |
| | | KNTMYLQMNNLKPDDTAIYYCAAGR | |
| | | DRYLRFSPDYWGQGTQVTVSS | |
| 750 | NBX08151 | QVQLQQSGGGLVQAGGSLRLSCAVS | NetB |
| | | GTIGSINPVAWSRQAPGKQREPIAF | |
| | | ISTAGATIYGDSAKGRFTISRDNAK | |
| | | NTVYLQMSNLKPEDSAVYYCNTESY | |
| | | RGQGTQVTVSS | |
| 751 | NBX08152 | QVQLQESGGGPVQAGGSLRLSCAYS | NetB |
| | | GRTFSGYGMGWFRQAAGKERTWVGT | |
| | | ITWNEGSTYYARSVRGRFTISRDNA | |
| | | KNTVYLQMDSLKPEDTAVYYCAAST | |
| | | AGYGLGSSPNEYEYWGQGTQVTVSS | |
| 752 | NBX08153 | QVQLQESGGGLVQAGDSLRLSCTAS | NetB |
| | | GRTFTRYGMGWFRQAPGKEREFVAG | |
| | | INGRGDSTSYGDSVKGRFTISRDDA | |
| | | KNTVYLQMNSLKLEDTAVYYCAVSF | |
| | | TSTFWFSPAQYDYWGQGTQVTVSS | |
| 753 | NBX08154 | QVQLQESGGGLVQAGGSLRLSCAVS | NetB |
| | | GSLFSFETMRWYRQAHGKQREWVAI | |
| | | ITTGASPAYADSVKGRFTISRDNAK | |
| | | NTVYLQMFSLKPEDTAVYYCYVRER | |
| | | VGSKEYWGQGTQVTVSS | |
| 754 | NBX08155 | QVQLQESGGGLVQPGGSLRLSCAAS | NetB |
| | | GLIFSASPMGWYRQAPGNQREFVAR | |
| | | ISASGMITKYADSVKGRFTVSRDDA | |
| | | KNTVYLQMNNLKPEDTAVYYCNAPR | |
| | | ANSDWPRENSWGQGTQVTVSS | |
| 755 | NBX08156 | QVQLQQSGGGLVQPGGSLRLSCAAS | NetB |
| | | GFTLSSYYIGWFRQAPGKEREGVSC | |
| | | FSNSDGNIAFADSVKGRFTVSRDNA | |
| | | KRMVYLQMTSLKLEDTAVYYCAVGY | |
| | | ACDYHELRQRFSSWGQGTQVTVSS | |
| 756 | NBX08157 | QVQLQQSGGGLVQPGGSLRLSCAAS | NetB |
| | | RSISSIQAMGWYRQAPGKORELVAR | |
| | | ITNGGTTRYADSVRGRFTISRDNAK | |
| | | NTVYLQMNSLKPEDTAVYYCNADER | |
| | | PYYGDAINSWGHGTQVTVSS | |
| 757 | NBX11001 | QVQLQQSGGGLVQPGGSLRLSCAAS | CnaA |
| | | GFAFDTSPMRWARQAPGKGLEWVST | |
| | | IFSDGSTHYVDSVKGRFTISRDNAK | |
| | | NMVFLQMNSLKTEDTAVYYCAMSGV | |
| | | RGQGTQVTVSS | |
| 758 | NBX11002 | QVQLQQSGGGLVQPGGSLRLSCAAS | CnaA |
| | | GFTLDTYAVGWFRQAPGKEREEVSC | |
| | | ISASGSMTTYAPFVKGRFTISKNNV | |
| | | RNMVDLQMNSLKPEDTAVYYCAAST | |
| | | RPLCSRGGNYDYWGQGTQVTVSS | |
| 759 | NBX11003 | QVQLQESGGGLVQPGGSLRLSCAAS | CnaA |
| | | GFTLDYSAIGWFRQAPGKEREDVSC | |
| | | ISSSGAYTSYVGSVKGRFSISRDNA | |
| | | KNTVFLQMNSLKPEDTAVYYCAARS | |
| | | GVCSRSSSDFGSWGQGTQVTVSS | |
| 760 | NBX11004 | QVQLQQSGPGLVKPSQTLSLTCTVS | CnaA |
| | | GGSITTNPYYWSWIRQPPGKGLEWI | |
| | | GYIDYSGSTYYSPSLRSRTTISRDT | |
| | | SKNQFTLQLSSVTPGDTAVYYCARS | |
| | | LRPVVTAKRDDEYWGQGTQVTVSS | |
| 761 | NBX11005 | QVQLQESGGGLVQPGGSLRLSCAAS | CnaA |
| | | GSIFSINAMGWFRQAPRKEREFVAT | |
| | | INWSGGITYYADSVKGRFTISRDNA | |
| | | KNTVYLQMNSLKPDDTAIYYCAADL | |
| | | RVGASGPISFAWAYDYWGQGTQVTV | |
| | | SS | |
| 762 | NBX11006 | QVQLQESGGGLVQPGGSLRLSCAAS | CnaA |
| | | GFSLDNYDIGWFRQAPGKEREGVGC | |
| | | ISRSGGTTTYAGSVSRRFTISRDNA | |
| | | KNTVYLQMNSLKPEDTGVYYCAPVD | |
| | | ITGPGRCPPARYEYGYWGQGTQGTA | |
| | | SS | |
| 763 | NBX11007 | QVQLQQSGGGLVQPGGSLRLSCAAS | CnaA |
| | | GFAFDRSAMRWARQAPGKGLEWVSS | |
| | | IFSDGSTHYVPSVEGRFTISRDNPK | |
| | | NMVYLQMNSLKPEDTAVYYCATSGI | |
| | | RGQGTQVTVSS | |
| 764 | NBX11008 | QVQLQESGGGLVQPGESLRLSCAAS | CnaA |
| | | GFAFDSSAMRWARQAPGKGLEWLCS | |
| | | IFKDGSTYYVDSVKGRFTISRDNAK | |
| | | NMVYLQMNSLKPEDTAVYYCATSGI | |
| | | RGQGTQVTVSS | |
| 765 | NBX11009 | QVQLQESGGGLVQAGGSLRLSCAAS | CnaA |
| | | GIGPNAIGWFRQAPGKERDFVAGIS | |
| | | RSGGYTNYAASVKDRFTISRDDAKN | |
| | | TVYLQMTNLSPEDTAIYTCAADAGN | |
| | | FPSRNPVTYGYWGQGTQVTVSS | |
| 766 | NBX11010 | QVQLQQSGGGLVQPGGSLRLSCAAS | CnaA |
| | | GFAFDRSPMRWARQAPGKGLEWISS | |
| | | IFSDGSTYYVDSVEGRFTISRDNDK | |
| | | NMVYLQMNSLKPEDTAVYYCATSGI | |
| | | RGQGTQVTVSS | |
| 767 | NBX11011 | QVQLQESGGGLVQPGGSLRLSCAAS | CnaA |
| | | GFTLDYYAIGWFRQAPGKEREGVSC | |
| | | ISSSGAYENYAGFVKGRFTISRDNA | |
| | | KNTVYLQMNNLKPEDTAVYYCAAAR | |
| | | GGCPPNTYYSGSSYFWEYDFWGQGT | |
| | | QVTVSS | |
| 768 | NBX11012 | QVQLQESGGGLVQPGGSLRLSCAAS | CnaA |
| | | GFNLNYYDIGWFRQAPGKEREGVSC | |
| | | ISRSGGTETYAPSVKGRFTISRDNA | |
| | | KNTVYLQMNSLEPEDTGVYYCAPVD | |
| | | ITGPGRCPPARYAYGSWGQGIQVTV | |
| | | SS | |
| 769 | NBX11013 | QVQLQESGGGLVQPGESLRLSCAAS | CnaA |
| | | GFAFDSSPMRWARQAPGKGLEWLCS | |
| | | IFKDGSTYYVDSVKGRFTISRDNAK | |
| | | NMVYLQMNSLKPEDTAVYYCATSGI | |
| | | RGQGTQVTVSS | |
| 770 | NBX11014 | QVQLQESGGGLVQPGESLRLSCAAS | CnaA |
| | | GFAFDSSPMRWARQAPGKGLEWMCT | |
| | | IFKDGTTYYVDSVKGRFTISRDNAK | |
| | | NMVYLQMNSLKPEDTAVYYCAMSGV | |
| | | RGQGTQVTVSS | |
| 771 | NBX11015 | QVQLQQSGGGLVQPGESLRLSCAAS | CnaA |
| | | GFAFDTSPMRWARQAPGKGLEWLCS | |
| | | IFKDGSTYYVDSVKGRFTISRDNAK | |
| | | NMVYLQMNSLKPEDTAVYYCATSGI | |
| | | RGQGTQVTVSS | |
| 772 | NBX11016 | QVQLQQSGGGLVQPGGSLRLSCAAS | CnaA |
| | | GFNLDNYHIGWFRQAPGKEREGVSC | |
| | | ISSSGGLTTYAPFVKGRFTISRDNA | |
| | | KNTVFLQMSSLKPEDTAVYYCGRVN | |
| | | YCARDMSAYDTWGQGTQVTVSS | |
| 773 | NBX11017 | QVQLQQSGGGLVQPGGSLRLSCAAS | CnaA |
| | | GFTRDYYTIGWFRQAPGKEREGVSC | |
| | | ISSSDGWTYYADSVKGRFTISRDNA | |
| | | KNTVYLQMNSLKPEDTAVYYCAAAY | |
| | | DTAGWGAGGMDYWGKGTLVTVSS | |
| 774 | NBX11018 | QVQLQQSGGGLVQAGGSLRLSCAAS | CnaA |
| | | GSIFSINAMGWFRQAPGKEREFVGA | |
| | | ITWSGGSTEYADHVKGRFTISRDNA | |
| | | KNTVYLQMNSLKPEDTAIYYCAADT | |
| | | RRWAGGSSWYGEEYDYWGQGTQVTV | |
| | | SS | |
| 775 | NBX11019 | QVQLQQSGGGLVQPGGSLRLSCEAS | CnaA |
| | | GFTLDYYTIGWFRQAPGKEREEVSC | |
| | | ISRSGAMTTYAGSVKGRFTISRDNA | |
| | | KKTVYLQMNSLKPEDTGIYYCAATV | |
| | | KSVCFRGELYDYWGQGTQVTVSS | |
| 776 | NBX11020 | QVQLQESGGGVVQPGGSLRLSCAAS | CnaA |
| | | EFSLANYAIGWFRQAPGKEREAVSC | |
| | | ISSSGNYMTYEPFVKGRFTISRDNA | |
| | | KNAVYLQMNSLEPEDTAVYYCARAR | |
| | | SGCSRNMYDSTDYWGQGTQVTVSS | |
| 777 | NBX11021 | QVQLQQSGGGLVQPGGSLTLSCAAS | CnaA |
| | | GFTLDYYAIGWFRQAPGKEREGVSC | |
| | | ISSSDGSTYYADSVKGRFTISRDNA | |
| | | KNTVYLQMNSLKPEDTAVYYCAAIG | |
| | | SGPLTAQGMCVMTRTPRDYDYWGQG | |
| | | TQVTVSS | |
| 778 | NBX11022 | QVQLQESGGGLVQPGGSLRLSCAAS | CnaA |
| | | GFAFDRSPMRWARQAPGKGLEWVSS | |
| | | SLPDGSTYYIDSVKGRFTISRDNVK | |
| | | NMVYLQMNSLKPEDTAVYYCATSGV | |
| | | RGQGTQVTVSS | |
| 779 | NBX11023 | QVQLQQSGGGLVQPGGSLRLSCAAS | CnaA |
| | | GFAFDRSAMRWARQAPGKGLEWVSS | |
| | | IFSDGTTHYVPSVEGRFTISRDNPK | |
| | | NMVYLQMNSLKPEDTAVYYCATSGI | |
| | | RGQGTQVTVSS | |
| 780 | NBX11024 | QVQLQESGGGLVQLGGSLRLSCAAS | CnaA |
| | | GFTFDSYVMTWVRQAPGKGLEWVSY | |
| | | ISQGGGATYYADSVKGRFTISRDNN | |
| | | KNTLYLQMNSLKPEDTAVYYCAKGG | |
| | | LADGTGFYGSPGALIRGQGTQVTVS | |
| | | S | |
| 781 | NBX11025 | QVQLQESGGGLVQPGGSLRLSCVAS | CnaA |
| | | GFTFNTYVMTWVRQAPGKGLEWVSY | |
| | | ISQGGAATYYTDSVKGRFTISRDNA | |
| | | KNTVYLQMNSLKPEDTAVYYCAQGG | |
| | | LADGSGFYGSPGALIRGQGTQVTVS | |
| | | S | |
| 782 | NBX11026 | QVQLQESGGGLVQPGGSLRLSCAAS | CnaA |
| | | GFTLDYYAIGWFRQAPGKEREGVSC | |
| | | IMSSDGYTYYADSVKGRFTISRDNA | |
| | | KNTVYLQMNSLKPEDTAVYYCAAAY | |
| | | DTAGWGADGMDYWGKGTLVTVSS | |
| 783 | NBX11027 | QVQLQESGGGLVQPGESLRLSCAAS | CnaA |
| | | GFAFDTSPMRWARQAPGKGLEWLCS | |
| | | IFEDGTTYYVDSVKGRFTISRDNAK | |
| | | NMVYLQMNSLKPEDTAVYYCATSGI | |
| | | RGQGTQVTVSS | |
| 784 | NBX11028 | QVQLQESGGGLVQPGGSLRLSCAAS | CnaA |
| | | GSIFSINGMGWFRQAPGKEREFVAA | |
| | | ISWSGSITYYADSVKGRFTISRDNA | |
| | | KNTVLLQMNSLKPEDTAIYYCAADI | |
| | | RVGASGPISFAWAYDYWGQGTQVTV | |
| | | SS | |
| 785 | NBX11029 | QVQLQESGGGLVQPGGSLRLSCAAS | CnaA |
| | | GFILDYYAIGWFRQAPGKEREGVSC | |
| | | ISSSDGSTYYADSVKGRFTISRDNA | |
| | | KNTVYLQMNSLKPEDTAVYYCAAVG | |
| | | SGPLSAQGMCVMTRTSRDADYWGQG | |
| | | TQVTVSS | |
| 786 | NBX11032 | QVQLQESGGGLVQPGGSLRLSCLAS | CnaA |
| | | GFTLDGYAIGWFRQAPGKEREGVSC | |
| | | ISPSGGMTTYAGFVKGRFTISRDNA | |
| | | KNTVYLQMNSLKPEDTGVYYCKVMP | |
| | | LCDRMWSPLGGSWGQGTQVTVSS | |
| 787 | NBX11033 | QVQLQESGGGLVQPGGSLRLSCAAS | CnaA |
| | | GFTLEAYAIGWFRQAPGKEREEVSC | |
| | | ISRSGGLTTYAGFVKGRFTISRDNA | |
| | | KNTAYLQMNSLNPEDTALYYCAAKQ | |
| | | TQCSRWNPEYEYWGQGTQVFVSS | |
| 788 | NBX11034 | QVQLQESGGGLVQPGGSLKLSCAAS | CnaA |
| | | GFTFEASAMRWARQAPGKGLEYVSS | |
| | | IFSDGTTYYVDSVKGRFTISRDNDK | |
| | | NMVYLQMNNLKPEDTAVYYCATSGI | |
| | | RGRGTQVTVSS | |
| 789 | NBX11035 | QVQLQESGPGLVKPSQTLSLTCTVS | CnaA |
| | | GGSITTNSYYWSWIRQPPGKGLEWI | |
| | | GYIDYSGSTYYSPSLRSRTTISRDT | |
| | | SKNQFTLQLSSVTPGDTAVYYCARS | |
| | | LRPVVTAKRDDEYWGQGTQVTVSS | |
| 790 | NBX11036 | QVQLQESGGGLVQLGGSLRLSCAAS | CnaA |
| | | GFTFDAYVMTWVRQAPGKGLEWVSY | |
| | | ISQGGAATYYADSVKGRFTISRDNN | |
| | | KNTLYLQMNSLKPEDTAVYYCAKGG | |
| | | LADGSGFYGSPGALIRGQGTQVTVS | |
| | | S | |
| 791 | NBX11037 | QVQLQESGGDLVQPGGSLRLSCAAS | CnaA |
| | | GFAFEASAMRWARQAPGKGLEYVSS | |
| | | IFSDGTTYYVDSVKGRFTISRDNDK | |
| | | NMVYLQMNNLKPEDTAVYYCATSGI | |
| | | RGRGTQVTVSS | |
| 792 | NBX11038 | QVQLQESGPGLVKPSQTLSLTCTVS | CnaA |
| | | GGSITTNSYYWSWIRQPPGKGLEWI | |
| | | GYIDYSGNTYYSPPLRSRTSISRDT | |
| | | SKNQFTLQLSSVTPEDTAVYYCARS | |
| | | VRPVVTAKGDNEYWGQGTQVTVSS | |
| 793 | NBX11039 | QVQLQESGGGLVQPGGSLRLSCAAS | CnaA |
| | | GFAFDTSPMRWARQAPGKGLEWVST | |
| | | SFSDGTTHYVDSVKGRFTISRDNAK | |
| | | NMVYLQMNSLKPEDTAVYYCATSGI | |
| | | RGQGTQVTVSS | |
| 794 | NBX11040 | QVQLQESGPGLVKPSQTLSLTCTVS | CnaA |
| | | GGSITTNTYYWSWIRQPPGKGLEWI | |
| | | GYIDYSGSTYYSPSLRSRTTISRDT | |
| | | SKNQFTLQLSSVTPEDTAVYYCARS | |
| | | VRPVVTAKSDDEYWGQGTQVTVSS | |
| 795 | NBX11041 | QVQLQESGPGLVKPSQTLSLTCTVS | CnaA |
| | | GGSITTNSYYWSWIRQPPGKGLEWI | |
| | | GYIDYSGNTYYSPPLRSRTSISRDT | |
| | | SKNQFTLQLSSVTPEDTAVYYCARS | |
| | | VRPVVTAKSDNEYWGQGTQVTVSS | |
| 796 | NBX11042 | QVQLQESGPGLVKPSQTLSLTCTVS | CnaA |
| | | GGSITTNSYYWSWIRQPPGKGLEWI | |
| | | GYIDYSGNTYYSPPLRSRTSISRDT | |
| | | SKNQFTLQLSSVTPEDTAVYYCARS | |
| | | VRPVVTAKRDNEYWGQGTQVTVSS | |
| 797 | NBX11043 | QVQLQESGGGLVQPGGSLRLSCAAS | CnaA |
| | | GFAFDSSAMRWARQAPGKGLEWVSS | |
| | | ILSDGSTYYVDSVKGRFTISRDNVK | |
| | | NMVYLQMNSLKPEDTAVYYCATSGI | |
| | | RGQGTLVTVSS | |
| 798 | NBX11044 | QVQLQESGGGLVQPGESLRLSCAAS | CnaA |
| | | GFAFDSSPMRWARQAPGKGLEWLCS | |
| | | IFGDGSTYYVDSVKGRFTISRDNAK | |
| | | NMVYLQMNSLKPEDTAVYYCAMSGV | |
| | | RGQGTQVTVSS | |
| 799 | NBX11045 | QVQLQESGGGLVQPGGSLRLACVGS | CnaA |
| | | GFTLDYYDIGWFRQAPGKEREGVSC | |
| | | IEGSGGSTNYADSVKGRFTISRDNA | |
| | | KNTVYLQMNSLKPDDTAVYYCAVAS | |
| | | SGSIGLCTVGRNAYNYWGQGTQVTV | |
| | | SS | |
| 800 | NBX11046 | QVQLQESGGGLVQPGRSLRLSCVAS | CnaA |
| | | GISRNAIGWFRQAPGKERDFVAGIS | |
| | | RSGGYTNYENFVKGRFTISRDDAKN | |
| | | TVYLQMTNLKPEDTAIYTCAADAGN | |
| | | FPSRNPVTYGYWGQGTQVTVSS | |
| 801 | NBX11047 | QVQLQQSGGGLVQPGGSLRLSCAAS | CnaA |
| | | GFTLDYYAIGWFRQAPGKEREGVSC | |
| | | ISSSDGSTYYADSVKGRFTISRDNA | |
| | | KNTVYLQMNSLKPEDTAVYYCAAVG | |
| | | SGPLSAQGMCVMTRTPRDYDYWGQG | |
| | | TQVTVSS | |
| 802 | NBX11048 | QVQLQESGGGLVQPGESLRLSCAAS | CnaA |
| | | GFAFDSSAMRWARQAPGKGLEYVCS | |
| | | IFEDGSTYYVDSVKGRFTISRDNAK | |
| | | NMVYLQMNSLKPEDTAVYYCTTSGI | |
| | | RGQGTQVTVSS | |
| 803 | NBX11049 | QVQLQQSGPGLVKPSQTLSLTCTVS | CnaA |
| | | GGSITTNHYYWSWIRQPPGKGLEWI | |
| | | GYIDYSGSTYYSPPLRSRTSISRDT | |
| | | SKNQFTLQLSSVTPEDTAVYYCARS | |
| | | VRPVVTAKRDDENWGQGTQVTVSS | |
| 804 | NBX11050 | QVQLQESGGGLVQPGESLRLSCAAS | CnaA |
| | | GFAFDKSAMRWARQAPGKGLEYVCS | |
| | | VFEDGSTYYVDSVKGRFTISRDNAK | |
| | | NMVYLQMNSLKPEDTAVYYCTTSGI | |
| | | RGQGTQVTVSS | |
| 805 | NBX11051 | QVQLQESGGGLVQPGGSLRLSCAAS | CnaA |
| | | GFTLEAYDIGWFRQAPGKEREEISC | |
| | | ISRSAGHTTYAGFVKGRFTISRDNA | |
| | | KNTAYLQMNSLNPEDTALYYCAAGQ | |
| | | TQCTRWSSEYEYWGQGTQVTVSS | |
| 806 | NBX11052 | QVQLQESGGGLVQPGESLRLSCAAS | CnaA |
| | | GFTFDRSPMRWARQAPGKGLEYLCT | |
| | | IFEDGTTYYVDSVKGRFTISRDNAK | |
| | | NMVYLQMNNLKPEDTAVYYCAMSGV | |
| | | RGQGTQVTVSS | |
| 807 | NBX11053 | QVQLQESGGGLVQPGGSLRLSCAAS | CnaA |
| | | GFTLENYAIGWFRQAPGKEREGVSC | |
| | | ISASGSTTTYEGFVKGRFTISRDNA | |
| | | KNTIYLQMNALEPEDTAVYYCARTH | |
| | | QLCPRARSPYDAWGQGTQVTVSS | |
| 808 | NBX11054 | QVQLQESGGGLVQPGGSLRLSCAAS | CnaA |
| | | GFTLDYYTIGWFRQAPGKEREGVSC | |
| | | IMTSDDYTYYADSVKGRFTISRDSA | |
| | | TNTVYLQMNSLKPEDTAVYYCAAAY | |
| | | DTAGWGADGMDYWGKGTLVTVSS | |
| 809 | NBX21001 | QVQLQESGGGLVQPGGSLRLSCAAS | NetB |
| | | GSIFSIKDMGWYRQPPGKQRELVAR | |
| | | ITTGGTTTYADSVKGRFTISRDNAK | |
| | | NTVYLQMNNLKPEDTDVYYCGVLLN | |
| | | YRGPSSASYHWGQGTQVTVSS | |
| 810 | NBX21002 | QVQLQESGGGLVQPGGSLRLSCAAS | NetB |
| | | RSIFSINVMGWYRQAPGKQRELVAH | |
| | | ITRGGSTYYYVDSVKGRFTISRDNA | |
| | | KTMVYLQMNSLKPEDTAVYYCNAVP | |
| | | ARDRWGQGTQVTVSS | |
| 811 | NBX21003 | QVQLQESGGGLVQPGGSLRLSCAAS | NetB |
| | | GFTFSRAAMIWVRQAPGKGLEWVSV | |
| | | IDSDGGTTRYADSVKGRFTISRDNA | |
| | | KNTLYLQMNNLKPEDTAVYYCSLFR | |
| | | GYSAYDLRSPGQGTQVTVSS | |
| 812 | NBX21004 | QVQLQESGGGLVQPGGSLRLSCAAS | NetB |
| | | RSIVSDNVMGWYRQAPGKQRELVAH | |
| | | ITRGGSTYYFVDSVKGRFTISRDNA | |
| | | KTMVYLQMNSLKPEDTAVYYCNTVP | |
| | | ARDRWGQGTQVTVSS | |
| 813 | NBX21005 | QVQLQESGGGLVQAGGSLRLSCAAS | NetB |
| | | GRTFSRYGMGWFRQAPGKDREFVAA | |
| | | ISWSGDSTYYADSVKGRFTISRDNA | |
| | | KNTVYLQMNSLKPEDTAVYYCAASY | |
| | | NPGTIATIRREYEYDYWGQGTQVTV | |
| | | SS | |
| 814 | NBX21006 | QVQLQESGGGLVQAGGSLRLSCAAS | NetB |
| | | GRTYSSYAMGWFRQAPGKEREFVAA | |
| | | ISWSGGSTYYADSVKGRFTISRDNA | |
| | | KNTVYLQMNSLKPEDTAVYYCAADR | |
| | | RTHVSDRLGEYDYWGQGTQVTVSS | |
| 815 | NBX21007 | QVQLQESGGGLVQPGGSLRLSCAAS | NetB |
| | | GRTFSSYAMGWFRQAPGKEREFVAA | |
| | | ISWSDEDTYYADSVKGRFTISRDNA | |
| | | ENTVYLQMNSLKPEDTAVYYCAADR | |
| | | SYTVMVRQMRGMDYWGKGTLVTVSS | |
| 816 | NBX21008 | QVQLQESG*GLVQPGGSLRLSCAAS | NetB |
| | | RSIVSINVMGWYRQAPGKQRELVAH | |
| | | ITRGGSTYYYVDSVKGRFTISRDNA | |
| | | KTMVYLQMNSLKPEDTAVYYCNTVP | |
| | | ARDRWGQGTQVTVSS | |
| 817 | NBX21009 | QVQLQESGGGLVQPGGSLRLSCAAS | NetB |
| | | RSIFSINVWGWYRQAPGKQRELVAH | |
| | | ITRGGSTYYYVDSVKGRFTISRDNA | |
| | | KTMVYLQMNSLKPEDTAVYYCNTVP | |
| | | ARDHWGQGTQVTVSS | |
| 818 | NBX21010 | QVQLQQSGGGLVQAGGSLRLSCAAS | NetB |
| | | GRTFSRYGMGWFRQAPGKEREFVAA | |
| | | ISWSGDSTYYADSVKGRFTISRDNA | |
| | | KNTVYLQMNSLKPEDMGVYYCAASY | |
| | | NPGNIATIRREYEYDYWGQGTQVTV | |
| | | SS | |
| 819 | NBX21011 | QVQLQESGGGLVQAGGSLRLSCAAS | NetB |
| | | GRTFSRYGMGWFRQAPGKEREFVAA | |
| | | ISWSGGSTYYADSVKGRFTISRDNA | |
| | | KNTVYLQMNSLKPEDTAVYYCAASY | |
| | | NPGNIATISREYEYDYWGQGTQVTV | |
| | | SS | |
| 820 | NBX21012 | QVQLQESGGGLVQAGGSLRLSCAAS | NetB |
| | | GRTFSRYGMGWFRQAPGKEREFVAA | |
| | | ISWSGGSTYYADSVKGRFTISRDNA | |
| | | KNTVYLQMNSLKPEDTAVYYCAASY | |
| | | NPGTIATIRREYEYDYWGQGTQVTA | |
| | | SS | |
| 821 | NBX21013 | QVQLQQSGGGLVQAGGSLRLSCAAS | NetB |
| | | GRTFSSYAMAWFRQAPGKEREFVAA | |
| | | IRSGNSTYYADSVKGRFTISRDNAK | |
| | | NTVYLQMNSLKPEDTAVYYCAADVV | |
| | | THLATRFYEYDYWGQGTQVTVSS | |
| 822 | NBX21014 | QVQLQESGGRLVQAGGSLRLSCAAS | NetB |
| | | GRTFSSYAMGWFRQAPGKEREFVAL | |
| | | ISWSGGSTYYADSVKGRFTISRDNA | |
| | | KNTVYLQMNSLKPEDTAVYYCAADR | |
| | | LTHYSDYPADFGSWGQGTQVTVSS | |
| 823 | NBX21015 | QVQLQQSGGGLVQAGGSLRLSCAAS | NetB |
| | | GRTFSSYAMGWFRQAPGKEREFVAL | |
| | | ISWSGSNTYYADSVKGRFTISRDNA | |
| | | KNTAYLQMNSLKPEDTAVYYCAADR | |
| | | LTHYSDYPADFDSWGQGTQVTVSS | |
| 824 | NBX21016 | QVQLQESGGGLVQPGGSLRLSCAAS | NetB |
| | | RSIFSANVMGWYRQAPGKQRELVAH | |
| | | ITRGSSTYYFLDSVKGRFTISRDNA | |
| | | KNIVYLQMNSLKPEDTAVYYCNLVP | |
| | | ARDRWGQGTQVTVSS | |
| 825 | NBX21023 | QVQLQESGGGLVQPGGSLRLSCAAA | NetB |
| | | GSIFSIRDMGWYRQAPGKQRERELV | |
| | | ATISSGGSTSYADFVKDRFTISRDN | |
| | | AKNTVYLQMNDLRPEDTAIYYCSVL | |
| | | TNPGWNRPLAYFWGQGTQVTVSS | |
| 826 | NBX21024 | QVQLQESGGGLVQAGGSLRLSCAAS | NetB |
| | | GPTFSSYAMYAMAWFRQAPGKQREF | |
| | | VASVSLTDGSKNYRDSVKGRFTISI | |
| | | DNANNTVYLQMNSLKPEDTAVYYCA | |
| | | GDLDSDVGYEHWGQGTQVTVSS | |
| 827 | NBX21025 | QVQLQESGGGLVQAGGSLRLSCAAS | NetB |
| | | GRTFSSYAMGWFRQAPGKEREFVAL | |
| | | ISWSGSNTYYADSVKGRFTISRDNA | |
| | | KNTAYLQMNSLKPEDTAVYYCAADR | |
| | | LTHYSDYPADFGSWGQGTQVTVSS | |
| 828 | NBX21026 | QVQLQQSGGGLVQAGGSLRLSCAAS | NetB |
| | | GRTFSRYGMGWFRQAPGKEREFVAA | |
| | | ISWSGGSTYYADSVKGRFTISRDNA | |
| | | KNTVYLQMNSLKPEDTAVYYCAASY | |
| | | NPGNIATIRREYEYDYWGQGTQVTV | |
| | | SS | |
| 829 | NBX21027 | QVQLQQSGGGLVQPGGSLRLSCAAS | NetB |
| | | RSIFSINVMGWYRQAPGKQRELVAH | |
| | | ITRGGSTYYYVDSVKGRFTISRDIA | |
| | | KTMVYLQMNSLKPEDTAVYYCNTVP | |
| | | ARDRWGQGTQVTVSS | |
| 830 | NBX21028 | QVQLQQSGGGSVQAGGSLRLSCAAS | NetB |
| | | GRAFSTYTMGWFRQAPGKEREFVAA | |
| | | ISWTGGSTYYADSVKGRFTISRDNA | |
| | | KAAIYLQMNSLKPEDTAVYYCAEKA | |
| | | RTAVDVRVTSGYDYWGQGTQVTVSS | |
| 831 | NBX21029 | QVQLQESGGGLVQPGGSLRLSCAAS | NetB |
| | | GRIFSINVMGWYRQAPGKQRELVAH | |
| | | ITRGGSTYYFVDSVKGRFTISRDNA | |
| | | KTMVYLQMNSLKPEDTAVYYCNTVP | |
| | | ARDRWGQGTQVTVSS | |
| 832 | NBX21030 | QVQLQESGGGLVQAGGSLRLTCAAS | NetB |
| | | GITFSSYAMSWFRQAPGKEREFVAA | |
| | | ISTSGGSTYYADSVKGRFTISRDNA | |
| | | KNTVYLQMNSLKPEDTAVYYCTARW | |
| | | DYPNTYEYDYSGQGTQVTVSS | |
| 833 | NBX21031 | QVQLQESGGGLVQPGGSLTLSCAAS | NetB |
| | | GGIFSFNVMGWYRQAPGKQRELVAH | |
| | | ITRGGSTYYYVDSVKGRFTISRDNA | |
| | | KTMVYLQMNSLKPEDTAVYYCNTVP | |
| | | ARDRWGQGTQVTVSS | |
| 834 | NBX21032 | QVQLQESGGGLVQAGESLKLSCTEA | NetB |
| | | SGHTFSSYIMGWFRRTPGKQREIVA | |
| | | GIRWSSGTTYYADSVKGRFTISRDN | |
| | | AKNTVYLQMNSLKPEDTAVYYCAAD | |
| | | AGRDSIYDYWGQGTQVTVSS | |
| 835 | NBX21033 | QVQLQESGGGLVQAGGSLRLSCAAS | NetB |
| | | GRTFSSYAMAWFRQAPGKEREFVAV | |
| | | IRSGDSTYYADSVKGRFTISRDNAK | |
| | | NTVYLQMNSLKPEDTAVYYCAADVV | |
| | | THIATRFYEYDYWGQGTQVTVSS | |
| 836 | NBX21034 | QVQLQQSGGGLVQAGGSLRLSCAAS | NetB |
| | | GRTFSSYAMGWFRQAPGKEREFVAL | |
| | | ISWSGASTYYADSVKGRFTISRDNA | |
| | | ENTVYLQMNSLKPDDTAVYYCAADR | |
| | | LTHYSDYPRDFGSWGQGTQVTVSS | |
| 837 | NBX21035 | QVQLQESGGGLVQPGGSLRLSCATS | NetB |
| | | RSIFRENVMGWYRQAPGKQRELVAH | |
| | | ITRGSSTYYFVDSVKGRFTISRDNA | |
| | | KNIVYLQMNSLKPEDTAVYYCNLVP | |
| | | ARDPWGQGTQVTVSS | |
| 838 | NBX21038 | QVQLQESGGGLVQAGGSLRLSCAAS | NetB |
| | | GRTFSMYTMGWFRQAPGKKREFVAT | |
| | | ISGSAGSTYYADSVKGRFTISRDTA | |
| | | KTTVYLQMNILKPEDTAVYYCAASE | |
| | | ATHGTNRQLDYDYWGQGTQVTVSS | |
| 839 | NBX21039 | QVQLQESGGGLVQAGGSLRLSCAAS | NetB |
| | | GGTFSRYGMGWFRQAPGKEREFVAA | |
| | | ISWSGDSTYYADSVKGRFTISRDNA | |
| | | KNTVHLQMNSLKPEDMGVYYCAASY | |
| | | NPGNIATIRREYEYDYWGQGTQVTV | |
| | | SS | |
| 840 | NBX21040 | QVQLQQSGGGLVQAGGSLRLSCAAS | NetB |
| | | GGTFSRYGMGWFRQAPGKEREFVAA | |
| | | ISWSGDSTYYADSVKGRFTISRDNA | |
| | | KNTVYLQMNSLKPEDTAVYYCAASY | |
| | | NPGTIATIRREYEYDYWGQGTQVTV | |
| | | SS | |
| 841 | NBX21041 | QVQLQESGGGLVQAGGSLRLSCVVS | NetB |
| | | RGTFSRYGMGWFRQAPGKEREFVAA | |
| | | ISWSGDSTYYADSVKGRFTISRDNA | |
| | | KNTVYLQMNSLKPEDTAVYYCAASY | |
| | | NPGTIPTIRREYEYDYWGQGTQVTV | |
| | | SS | |
| 842 | NBX21042 | QVQLQESGGGLVQAGGSLRLSCAAS | NetB |
| | | GRTFSRLGMGWFRQAPGKEREFVAA | |
| | | ISWSGDMTYYADSVKGRFTISRDNA | |
| | | KNTVYLQMNSLKPEDTAVYYCGASY | |
| | | NPGTIATIRREYEYDYWGQGTQVTV | |
| | | SS | |
| 843 | NBX21043 | QVQLQESGGGLVQAGGSLRLSCAAS | NetB |
| | | GGTFSRYGMGWFRQAPGKEREFVAA | |
| | | ISWSGDSKYYEDSVKGRFTISRDNA | |
| | | KNTVYLQMNSLKPEDTAVYYCAASY | |
| | | NPGNIATIRREYEYDYWGQGTQVTV | |
| | | SS | |
| 844 | NBX21044 | QVQLQESGGGLVQAGGSLRLSCAAS | NetB |
| | | GGTFSRYGMGWFRQAPGKEREFVAA | |
| | | ISWSGDSTYYADSVKGRFTISRDNA | |
| | | KNTVYLQMNNLKPEDTAVYYCAASY | |
| | | NPGNIATIRREYEYDYWGQGTQVTV | |
| | | SS | |
| 845 | NBX21045 | QVQLQESGGGLVQPGGSLRLSCAAS | NetB |
| | | GSSGSIKDMGWYRQPPGKQRELVAR | |
| | | ISTGGTTTYADSVKGRFTISRDNAK | |
| | | NTVYLQMNNLKPEDTDVYYCSVLLN | |
| | | IRGPSSASYHWGQGTQVTVSS | |
| 846 | NBX21046 | QVQLQQSGGGLVQPGGSLRLSCAAS | NetB |
| | | GSSGSIKDMGWYRQPPGKQRELVAR | |
| | | ITTGGTTTYADSVKGRFTISRDNAK | |
| | | NTVYLQMNNLKPEDTDVYYCNVLLN | |
| | | YRGPSSASYHWGQGTQVTVSS | |
| 847 | NBX21047 | QVQLQESGGGLVQAGGSLRLSCVAS | NetB |
| | | GRSFSSYTMGWFRQAPGKEREFVTS | |
| | | VTWNGEVPYYADSVKGRFTISRDNA | |
| | | KNTVYLRMNSLKPEDSAVYYCAAGN | |
| | | PGRGYDYWGQGTQVTVSS | |
| 848 | NBX21048 | QVQLQESGGGLVQAGGSLRLSCAAS | NetB |
| | | GGTFSRYGMGWFRQAPGKEREFVAA | |
| | | ISWSGGSTYYADSVKGRFTISRDNA | |
| | | KNTVSLQMNSLKPEDTAVYYCAASY | |
| | | NPGNIATIRREYEYDYWGQGTQVTV | |
| | | SS | |
| 849 | NBX21049 | QVQLQESGGGLVQAGGSLRLSCAAS | NetB |
| | | GRTFSNYAMGWFRQAPGKEREFVAA | |
| | | ISRSGGSTYYADSVKGRFTISRDNA | |
| | | KNTVYLQMNSLKPEDTAVYYCAADS | |
| | | LRTHVSDRSYEYDSWGQGTQVTVSS | |
| 850 | NBX21050 | QVQLQESGGGLVQAGGSLRLSCAAS | NetB |
| | | GGTFSRYGMGWFRQAPGKEREFVAA | |
| | | ISWSGDNTYYADSVKGRFTISRDNA | |
| | | KNTVYLQMNSLKPEDMGVYYCAASY | |
| | | NPGNIATIRREYEYDYWGQGTQVTV | |
| | | SS | |
| 851 | NBX21051 | QVQLQESGGGLVQAGGSLRLSCAAS | NetB |
| | | GRTFSSYAMGWFRQAPGKEREFVAA | |
| | | ISRSGASTYYADSVKGRFTISRDNA | |
| | | KNTVYLQMDNLKPEDTAVYYCAADS | |
| | | LRTHVSHVSYEYDSWGQGTQVTVSS | |
| 852 | NBX21052 | QVQLQESGGGLVQAGGSLRLSCAAS | NetB |
| | | GRTFSSYAMGWFRQGPGKEREFVAA | |
| | | IARSGSSTYYADSVKGRFTISRDKA | |
| | | KNTVYLQMNSLKPEDTAVYYCAADS | |
| | | LRTHVSDRSDEYDYWGQGTQVTVSS | |
| 853 | NBX21053 | QVQLQESGGGLVQAGGSLRLSCAAS | NetB |
| | | GRTFSRYGMGWFRQAPGKDREFVAA | |
| | | ISWSGDSTYYVDSVKGRFTISRDNA | |
| | | KNTVYLQMNSLKPDDTAVYYCGASY | |
| | | NPGNIATIRREYEYDYWGQGTQVTV | |
| | | SS | |
| 854 | NBX21054 | QVQLQQSGGGLVQAGDSLRLSCAAS | NetB |
| | | GRTFSHYAMGWFRQAPGKEREFVAA | |
| | | IRTSAGITRYGDSAEGRFTISSDNA | |
| | | KNTVYLQMNSLKPEDTAVYYCAIDD | |
| | | DYAYYPPSLLDRYDYWGQGTQVTVS | |
| | | S | |
| 855 | NBX21055 | QVQLQESGGGLVQAGGSLRLSCAAS | NetB |
| | | GRTASSAAMGWFRQAPGKEREFVAA | |
| | | ISWSGGSTYYADSVKGRFTISRDNA | |
| | | KNTVYLQMNSLKPEDTAVYYCAADR | |
| | | RTHVSDRLGEYDYWGQGTQVTVSS | |
| 856 | NBX21056 | QVQLQESGGGLVQAGGSLRLSCAAS | NetB |
| | | GRTFSTYAMGWFRQAPGKEREFVAA | |
| | | ISRSGASTYYGDSVKGRFTISRDNA | |
| | | KNTVYLQMDSLKPEDTAVYYCAADS | |
| | | LRTHVSHVSYEYDSWGQGTQVTVSS | |
| 857 | NBX21057 | QVQLQESGGGLVQAGGSLRLSCAAS | NetB |
| | | GRTLSRYGMGWFRQAPGKEREFVAA | |
| | | ISWSGDRTYYADSVNGRFTISRDNA | |
| | | KNTVYLQMNSLKPEDMGVYYCAASY | |
| | | NPGNIATIRREYEYDYWGQGTQVTV | |
| | | SS | |
| 858 | NBX21068 | QVQLQESGGGLRQAGGSLRLSCAAS | NetB |
| | | GGTFSRYGLGWFRQAPGKEREFVAA | |
| | | ISWSGDRTYYADSVKGRFTISRDNA | |
| | | KNTVYLQMNSLKPEDTAVYYCGASY | |
| | | NPGNIATIRREYEYDYWGQGTQVTV | |
| | | SS | |
| 859 | NBX21069 | QVQLQQSGGGLVQAGGSLRLSCAAS | NetB |
| | | EGTFSRYGMGWFRQAPGKEREFVAA | |
| | | ISWSGDSTYYADSVKGRFTISRDNA | |
| | | KNTVYLQMNSLKPEDTAVYYCAASY | |
| | | NPGNIATIRREYEYDYWGQGTQVTV | |
| | | SS | |
| 860 | NBX21070 | QVQLQESGGGLVQAGGSLRLSCAAS | NetB |
| | | GRTFSMYTMGWFRQAPGKKREFVAT | |
| | | ISGSAGSTYYADSVKGRFTISRDTA | |
| | | KTTVYLQMNILKPEDTAVYYCASST | |
| | | ATHGTNRQLDYDYWGQGTQVTVSS | |
| 861 | NBX21071 | QVQLQESGGGLVQAGGSLRLSCAPS | NetB |
| | | GRTFSTGAMGWFRQAPGKEREFVAA | |
| | | ISWSGGSTYYADSVKGRFTISRDNA | |
| | | KNTVYLQMNSLKPEDTAVYYCAASL | |
| | | LTHVSDRALEYDYWGQGTQVTVSS | |
| 862 | NBX21072 | QVQLQQSGGGLVQAGGSLRLSCAAS | NetB |
| | | EGTFSRYGMGWFRQAPGKEREFVAA | |
| | | ISWSGDRTYYTDSVKGRFTISRDNA | |
| | | KNTVYLQMNSLKPEDTAVYYCAASY | |
| | | NPGNIATIRREYEYDYWGQGTQVTV | |
| | | SS | |
| 863 | NBX21073 | QVQLQESGGGLVQPGGSLRLSCAAS | NetB |
| | | GSIFSINVMGWYRQAPGKQRELVAA | |
| | | ATSGGQTKYAHSVKGRFTISRDNAK | |
| | | NTVYLQMNSLKPEDTAVYYCNGRSA | |
| | | SYYPTYDYWGQGTQVTVSS | |
| 864 | NBX21074 | QVQLQLQESGGGLVQAGDSLRLSCA | NetB |
| | | ASGRTFSHYAMGWFRQAPGKEREFV | |
| | | AAIRGNAGITRYADSAEGRFTISTD | |
| | | NAKNTVYLQMNSLEPEDTAVYYCAI | |
| | | DLDYAYYPPSLVDRYDYWGQGTQVT | |
| | | VSS | |
| 865 | NBX21075 | QVQLQESGGGLVQPGGSLRLSCAAS | NetB |
| | | GSSGSMKDMGWYRQPPGKQRELVAR | |
| | | ITTGGTTTYADSVKGRFTISRDNAK | |
| | | NTVYLQMNNLKPEDTDVYYCNVLLN | |
| | | YRGPSSASYHWGQGTQVTVSS | |
| 866 | NBX21076 | QVQLQESGGGLVQAGGSLRLSCAAS | NetB |
| | | GRTFSHYAMGWFRQAPGKEREFAAA | |
| | | ISRSGGSTYYADSVKGRFTISRDNA | |
| | | KNTVYLQMNSLKPEDTAVYYCAADS | |
| | | LRTHVSDRSYEYDSWGQGTQVTVSS | |
| 867 | NBX21077 | QVQLQESGGGLVQAGGSLRLSCAAS | NetB |
| | | GRTFSTYAMGWFRQAPGKEREFVAA | |
| | | ISRSGGSTYYADSVKGRFTISRDKA | |
| | | KNTVYLQMNSLKPEDTAVYYCAADS | |
| | | LRTHVSDRSYEYDYWGQGTQVTVSS | |
| 868 | NBX21078 | QVQLQESGGGLVQPGGSLRLSCAAS | NetB |
| | | GSIFSSDDMGWYRQAPGKQRELVAL | |
| | | ITSGGMTKYEDFVKGRFTISRDNSK | |
| | | DTVSLQMNSLKPEDTAVYYCNAFRS | |
| | | KVVDGIILKRRDYDYWGQGTQVTVS | |
| | | S | |
| 869 | NBX21079 | QVQLQQSGGGLVQAGGSLRLSCAAS | NetB |
| | | GRTFSSYTMGWFRQAPGKEREFVAT | |
| | | ISGSAGSTYYADSVKGRFTISRDTA | |
| | | KTTVYLQMNILKPEDTAVYYCAASE | |
| | | ATHGTNRQLDYDYWGQGTQVTVSS | |
| 870 | NBX21080 | QVQLQESGGGLVQAGGSLRLSCAAS | NetB |
| | | GSIFSDDIWGWYRQAPGKQRELVAH | |
| | | ITRGSRPYYYVDSVKGRFTISRDNA | |
| | | KTMVYLQMNSLKPEDTAVYYCNAVP | |
| | | ARDRWGQGTQVTVSS | |
| 871 | NBX21081 | QVQLQQSGGGLVQPGGSLRLSCAAS | NetB |
| | | GSIGSINVMGWYRQAPGNERELVAH | |
| | | VTSGGMTRYTESVKGRFTISRDGAK | |
| | | NTVYLQMNSLKPEDTAVYYCNGRSA | |
| | | SYYPTYNYWGQGTQVTVSS | |
| 872 | NBX21082 | QVQLQQSGGGLVQAGGSLRLSCAAS | NetB |
| | | GDTLSRYGMAWFRQAPGKEREFVAA | |
| | | ITWSGSSTYYADSVKGRFTISRDNA | |
| | | KNTVDLQMNSLKPKDTAVYYCAAGP | |
| | | NLGPITIMRDYEYDYWGQGTQVTVS | |
| | | S | |
| 873 | NBX21083 | QVQLQQSGGGLVQPGGSLRLSCAAS | NetB |
| | | GSIFSINVMGWYRQAPGKQRELVAS | |
| | | ITSGGKSRYADSVKGRFTISRDNGQ | |
| | | NTVYLQMNSLKPEDTANYYCNGRSA | |
| | | SYYPTYDYWGQGTQVTVSS | |
| 874 | NBX21084 | QVQLQQSGGGLVQPGGSLRLSCAAS | NetB |
| | | GSIGSINVMGWYRQAPGNERELVAH | |
| | | ATSGGMTRYTDSVKGRFTISRDGAK | |
| | | NTVYLQMNNLKPEDTAVYYCNGRSA | |
| | | SYYPTYNYWGQGTQVTVSS | |
| 875 | NBX21085 | QVQLQQSGGGLVQAGGSLRLSCAAS | NetB |
| | | GGTFSRYGMGWFRQAPGKEREFVAA | |
| | | ISWSGDRTYYADSVKGRFTISRDNA | |
| | | KDTVYLQMNSLKPEDTAVYYCAASY | |
| | | NPGNIATIRREYEYDYWGQGTQVTV | |
| | | SS | |
| 876 | NBX21086 | QVQLQESGGGLVQAGGSLRLSCVVS | NetB |
| | | GSTFSINRVAWFRQAPGKERQFVAG | |
| | | IRSNGITNYADSVKGRFTISSDNEK | |
| | | NTVYLQMNSLKPEDTAIYYCNDPLA | |
| | | PFSSDASWGQGTQVTVSS | |
| 877 | NBX22019 | QVQLQQSGGGLVQAGGSLRLSCAAS | CnaA |
| | | GRTFSSYAMGWFRQAPGKEREFVAS | |
| | | ISWSGGITWYADSVKGRFTISRDNA | |
| | | KNTVYLQMNSLKPEDTAVYYCAADA | |
| | | RSDSGSYYYPADFGSWGQGTQVTVS | |
| | | S | |
| 878 | NBX22020 | QVQLQESGGGLVQAGGSLRLSCAAS | CnaA |
| | | GGTFSSYPMAWFRQAPGKEREFVAA | |
| | | ISWSGGTTYYADSVKGRFTISRDNA | |
| | | KNTVYLQMNSLKPEDTAVYYCAAQS | |
| | | GPYGSSSSWEADFGSWGQGTQVTVS | |
| | | S | |
| 879 | NBX22021 | QVQLQESGGGLVQAGGSLRLSCTAS | CnaA |
| | | GRTLSSYIMAWFRQAPGKEREFVAA | |
| | | IAWSGSVTQYADSVKGRFTISRDNA | |
| | | KNTAYLQMNSLKPEDTAVYYCAGDL | |
| | | HGIGYEYKYWGQGTQVTVSS | |
| 880 | NBX22022 | QVQLQESGGGLVQSGGSLRLSCAAS | CnaA |
| | | GFSLDDYAIGWFRQAPGKEREGVSC | |
| | | ISRTSDGSTYFADSVKGRFTISSDN | |
| | | AKNTVYLQMNSLKPEDTAVYYCAAG | |
| | | FVCSGYGGGIRGYEYDYWGQGTQVT | |
| | | VSS | |
| 881 | NBX22023 | QVQLQESGGGLVQPGGSLRLSCAFS | CnaA |
| | | GNIFSINLMTWYRQAPGKQREFVAS | |
| | | ITSGGNTNYVNFAKGRFTISTDNAK | |
| | | NTMYLQLNSLOPEDTAVYYCHCDWC | |
| | | SWGQGTQVTVSS | |
| 882 | NBX22024 | QVQLQESGGGLVQAGGSLRLSCAAS | CnaA |
| | | GGTFSSYPMGWFRQAPGKEREFVAA | |
| | | ISWSGGGTYYADSVKGRFTISRDNA | |
| | | KNTVYLQMNSLKPEDTAVYYCAADS | |
| | | PVGPSGLTTHWGYWGQGTQVTVSS | |
| 883 | NBX22025 | QVQLQESGGGLVQAGGSLRVSCAAS | CnaA |
| | | GRTFSSYVMGWFRQAPGKEREFVAA | |
| | | ISWSGGVTYYADSVKGRFTISRDNA | |
| | | KNTVYLQMNSLKPDDTAVYYCAADA | |
| | | RSDSGRYYYPADFGSWGQGTQVTVS | |
| | | S | |
| 884 | NBX22029 | QVQLQQSGGGLVQAGGSLRLSCAAS | CnaA |
| | | GRTFSNYVMGWFRQPPGKEREFVAA | |
| | | ISWSGGTTSYADSVKGRFTISRDNA | |
| | | KNTVYLQMNSLKPDDTAVYYCAAEH | |
| | | GAGSRWYFPEDFGSWGQGTQVTVSS | |
| 885 | NBX22030 | QVQLQESGGGLVQPGGSLRLSCAFS | CnaA |
| | | GNIFSINLMTWYRQAPGKQREFVAS | |
| | | ITSSGNTNYVNSGKGRFTISTDNAK | |
| | | NTMYLQLNSLOPEDTAVYYCHCDWC | |
| | | SWGQGTQVTVSS | |
| 886 | NBX22031 | QVQLQESGGGLVQAGGSLRLSCAAS | CnaA |
| | | GGTFSSYPMGWFRQAPGKEREFVAA | |
| | | ISWSGGVTYYADSVKGRFTISRDNA | |
| | | KNTVYLQMNSLKPEDTAVYYCAAQS | |
| | | GPYGSSSSWEADFDSWGQGTQVTVS | |
| | | S | |
| 887 | NBX22032 | QVQLQESGGGLVQAGGSLRLSCVAS | CnaA |
| | | GRTFSSYVMAWFRQAPGKEREFVAA | |
| | | ISWSGGVTNYADSVKGRFIISRDNA | |
| | | KNTVYLQMNSLKPEDTAVYYCAAEV | |
| | | GYGSRWYYETDFGSWGQGTQVTVSS | |
| 888 | NBX22033 | QVQLQQSGGGLVQAGGSLRLSCAAS | CnaA |
| | | GRTLSSYAMGWFRQAPGKEREFVAA | |
| | | ISWSGGVTHYEDSVKGRFTISRDNA | |
| | | KNTVYLQMNSLKPEDTAVYYCAALP | |
| | | DGRSWYQADYWGQGTQVTVSS | |
| 889 | NBX22034 | QVQLQQSGGGLVQAGGSLRLSCAAS | CnaA |
| | | GRTFSSYAMGWFRQAPGKEREFVAA | |
| | | ISWSGGVTHYADSVKGRFTISRDNA | |
| | | KNTVYLQMNSLKPEDTAVYYCAAEN | |
| | | GYGSSWYFEADFGSWGQGTQVTVSS | |
| 890 | NBX22035 | QVQLQQSGGGLVQAGGSLRLSCAAS | CnaA |
| | | GRTFSGSGMGWFRQAPGKEREFVAA | |
| | | ISWTGSITYYADSVKGRFTISRENA | |
| | | KNTVYLQMNSLKPEDTAVYYCARAT | |
| | | MGPTSRSDAYDYWGQGTQVTVSS | |
| 891 | NBX22036 | QVQLQESGGGLVQPGGSLRLSCAFS | CnaA |
| | | GNIFSINVMSWYRQAPGKQREFVAD | |
| | | ITSGGNTNYVNFGKGRFTISTDNAK | |
| | | NTMYLQLNSLOPEDTAVYYCHCDWC | |
| | | SWGQGTQVTVSS | |
| 892 | NBX22037 | QVQLQQSGGGLVQPGGSLRLSCAFS | CnaA |
| | | GNIFSINVMSWYRQAPGKQREFVAD | |
| | | ITSGGDTNYVNSGKGRFTISTDNAK | |
| | | NTMYLOLNSLQPEDTAVYYCHCDWC | |
| | | SWGQGTQVTVSS | |
| 893 | NBX22038 | QVQLQESGGGLVQPGGSLRLSCAFS | CnaA |
| | | GNIFSINLMTWYRQAPGKQREFVAS | |
| | | ITSGGHTNYVNSGKGRFTISTDNAK | |
| | | NTMYLQLNSLOPEDTAVYYCHCDWC | |
| | | SWGQGTQVTVSS | |
| 894 | NBX22039 | QVQLQESGGGLVQPGDSLRLSCAAS | CnaA |
| | | GGTFSNYAMGWFRQAPGKEREFVAA | |
| | | ISWSGGVAHYADSVKGRFTISRDNA | |
| | | KNTVYLQMNSLKPEDTAVYTCAAEV | |
| | | GYGTSWYYEADFGSWGQGTQVTVSS | |
| 895 | NBX22040 | QVQLQESGGGLVQAGGSLRLSCAAS | CnaA |
| | | GRTFSGSGMGWFRQAPGKEREFVAV | |
| | | ISWTGSITYYADSVKGRFTISRENA | |
| | | KNTVYLQMNSLKPEDTAVYYCARST | |
| | | VGPTSRSDAYDYWGQGTQVTVSS | |
| 896 | NBX22041 | QVQLQESGGGLVQAGGSLRLSCTAS | CnaA |
| | | GRTLSDYIMGWFRQAPGKEREFVAA | |
| | | IAWSGSTTQYTDSVKGRFTISRDNA | |
| | | KNTAYLQMNSLKPEDTAVYYCAGDL | |
| | | HGIGYEYKYWGQGTQVTVSS | |
| 897 | NBX22042 | QVQLQESGGGLVQAGGSLRLSCAAS | CnaA |
| | | GRTFSGSPMGWFRQAPGKEREFVAV | |
| | | ISWTGSITYYADSVKGRFTISRENA | |
| | | KNTVYLQMNSLKPEDTAVYYCARST | |
| | | VGPTSRSDAYDYWGQGTQVTVSS | |
| 898 | NBX22043 | QVQLQESGGAVVQPGGSLRLSCAFS | CnaA |
| | | GNVLSINLMTWYRQAPGKQREFVAS | |
| | | ITSGGDTNYVNFGKGRFTIATDNAK | |
| | | NMMYLQLNSLOPEDTAVYYCHCDWC | |
| | | SWGQGTLVTVSS | |
| 899 | NBX22044 | QVQLQESGGGLVQAGGSLRLSCVAS | CnaA |
| | | GRAFSRYTMGWFRQAPGKEREFAAT | |
| | | IDWSGGIDWSGGRSTFYADSVKGRF | |
| | | TISRDNAENTVYLQMNSLKPEDTAV | |
| | | YYCAAIDYPGTRPAVSANEYDYWGQ | |
| | | GTQVTVSS | |
| 900 | NBX22045 | QVQLQESGGGLVQAGGSLRLSCTAS | CnaA |
| | | GRTLSNYIMAWFRQAPGREREFVAA | |
| | | IAWSGSVTQYADSVKGRFTISRDNA | |
| | | KNTAYLQMNSLKPEDTAVYYCAGDL | |
| | | HGIGYEYAYWGQGTQVTVSS | |
| 901 | NBX22046 | QVQLQESGGGLVQAGGSLRLSCTAS | CnaA |
| | | GRTLSSYIMAWFRQAPGKEREFVAA | |
| | | IGWSGSTTQYADSVKGRFTISRDNA | |
| | | KNTAYLQMNALKPEDTAVYYCAGDL | |
| | | QGIGYEYHNWGQGTQVTVSS | |
| 902 | NBX22055 | QVQLQESGGGLVQAGGSLRLSCAAS | CnaA |
| | | GGTFSNYVMGWFRQAPGKEREFVAA | |
| | | ISWSGGSTYYADSVKGRFTISRDNA | |
| | | KNTVYLQMNSLKPEDTAVYYCAAET | |
| | | GGGSRWYRTADFGSWGQGTQVTVSS | |
| 903 | NBX22056 | QVQLQQSGGGLVQAGGSLTLSCAAS | CnaA |
| | | GRTFSTYGMGWFRQAPGKEREFVAA | |
| | | ISASGGGTAYANSVKARFTISRDNA | |
| | | KNTVYLQMNSLKPEDTAVYYCAADR | |
| | | TTWGRQTPYDYWGQGTQVTVSS | |
| 904 | NBX22057 | QVQLQESGGGLVQAGGSLRLSCAAS | CnaA |
| | | GRTFSTYAMGWFRQAPGKEREFVTR | |
| | | IRWNTGSTDYADSVKGRFTISRDKA | |
| | | KNMVWLQMDSLKPEDTAVYYCALKR | |
| | | YGGGTSTYDQGYDTWGQGTQVTVSS | |
| 905 | NBX22058 | QVQLQQSGGGLVQAGGSLRLSCAAS | CnaA |
| | | GRTFSSGAMGWFRQAPGKEREFAAA | |
| | | ISRSGSSTLYADSVKGRFTITRDSA | |
| | | KSTVFLQMNSLKPEDTAVYYCAASL | |
| | | GRAYETSASGAYDYWGQGTQVTVSS | |
| 906 | NBX22059 | QVQLQESGGGLVQTGDSLRLSCAAS | CnaA |
| | | GRTFRSYGVAWFRQAPGKERDFVAS | |
| | | ISSSGGGTSYAGSVKDRFAIFRDNA | |
| | | KNTVYLQMNSLKPEDTAVYYCAGAV | |
| | | RDWGRDAIYDSWGQGTQVTVSS | |
| 907 | NBX22060 | QVQLQESGGGLVQPGGSLRLSCAAS | CnaA |
| | | GFTFSRYAMSWYRQAPGKERELVAR | |
| | | ITSAGGSTNYADSVKGRFTISRDNA | |
| | | KNTVYLQMNSLKPEDTAVYYCNAEM | |
| | | YSNADVMFNGYWGQGTQVTVSS | |
| 908 | NBX22061 | QVQLQESGGGLVQAGGSLRLSCAAS | CnaA |
| | | GRTISNYTMGWFRQAPGKEREFVAG | |
| | | INPSGGATYYAPFVKDRFTISRDSA | |
| | | KNTVYLQMNSLKPEDTAVYSCAAGS | |
| | | SHGRRWYIDGGRYDYWGQGTQVTIS | |
| | | S | |
| 909 | NBX22062 | QVQLQESGGGLVQPGGSLRLSCAAS | CnaA |
| | | GFTFSNYAMSWYRQAPGKERELVAR | |
| | | ITSTGGSTNYADSVKGRFTISRDNA | |
| | | KNTVYLQMNSLKPEDTAVYYCNAEI | |
| | | YTHYDVMFNGYWGQGTQVTVSS | |
| 910 | NBX22063 | QVQLQESGGGSVQAGGSLRLSCAAS | CnaA |
| | | GRTFSTYGMGWFRQAPGKEREFVAG | |
| | | ISASGGGTAYVSSVKARFTISRDNA | |
| | | KNTVYLQMNSLKPEDTAIYYCAAAR | |
| | | TGWGRQTTHDYWGQGTQVTVSS | |
| 911 | NBX22064 | QVQLQQSGGGLVQPGGSLRLSCKAS | CnaA |
| | | GFTFSSYSMRWYRQIPENEREFVAF | |
| | | ISSGGGDITSVADSVKGRFTITRDD | |
| | | TRNTVYLQMNSLKPEDTAVYYCMRV | |
| | | SGDYWGQGTQVTVSS | |
| 912 | NBX22065 | QVQLQESGGGLVQAGGSLRLSCTAS | CnaA |
| | | GRTFSSGAMGWFRQAPGKEREFVAA | |
| | | ISRSGSGTLYADSVKGRFTITRVSA | |
| | | KSTVFLQMNSLNPEDTAVYYCAASL | |
| | | GRAYETSASGAYDYWGQGTQVTVSS | |
| 913 | NBX22066 | QVQLQQSGGGLVQAGGSLRLSCTAS | CnaA |
| | | GRTFRSYGVGWFRQAPGKERDFVAG | |
| | | ISWSGGGTAYAGSVKDRFAIFRDNA | |
| | | KSTVYLQMNSLKPEDTAVYYCAGAV | |
| | | GDWGRDAIYDYWGQGTQVTVSS | |
| 914 | NBX22067 | QVQLQQSGGGLVQAGGSLRLSCAAS | CnaA |
| | | GRTFSSYGMGWFRQVPGKEREFVTG | |
| | | ISTSGGGTSYANFVNDRFTISRDNA | |
| | | KNTVYLQMNSLKPGDTAVYYCAASS | |
| | | RGWGRGVSYDYWGQGTQVTVSS | |
| 915 | NBX22068 | QVQLQQSGGGLVQAGGSLRLSCAAS | CnaA |
| | | GGTFSNYVMGWFRQAPGKEREYVAA | |
| | | ISWSGGNAIYADSVKGRFTISRDNA | |
| | | KNTVYLQMDRLKPEDTALYYCAAET | |
| | | GYGNRWYAPADFGSWGQGTQVTVSS | |
| 916 | NBX22069 | QVQLQESGGGSVQAGGSLRLSCAAS | CnaA |
| | | GHTFSTYGMGWFRQAPGKEREFVAG | |
| | | ISASGGGTAYVSSVKARFTISRDNA | |
| | | KNTVYLQMNSLKPEDTAIYYCAAAR | |
| | | TGWGROTTHDYWGQGTQVTVSS | |
| 917 | NBX22070 | QVQLQQSGGGLVQAGGSLRLSCAAS | CnaA |
| | | GRTFSSYGMGWFRQAPGKEREFVTG | |
| | | ISRSGGGTSFAPFVKGRFTISRDNA | |
| | | KNTVYLQMNSLKPGDTAIYYCAASS | |
| | | TGWGREDSYDYWGQGTQVTVDS | |
| 918 | NBX22083 | QVQLQESGGGLVQAGGSLRLSCAAS | CnaA |
| | | GRTFSSYVMGWFRQAPGKEREFVAA | |
| | | ISWSGGVTYYADSVKGRFTISRDNA | |
| | | KNTMYLQMNSLKPEDTAVYYCAASD | |
| | | SENSGSYYRNQDFGYWGQGTQVTVS | |
| | | S | |
| 919 | NBX22084 | QVQLQQSGGGLVQAGGSLRLSCAAS | CnaA |
| | | GRTFSNYAMGWFRQAPGKEREFVAA | |
| | | ISWSGGITWYADSVKGRFTISRDNA | |
| | | KNTVYLQMNSLKPEDTAVYYCAADR | |
| | | GTDSGSYYYTEDFGSWGQGTQVTVS | |
| | | S | |
| 920 | NBX22085 | QVQLQESGGGLVQAGGSLRLSCAAS | CnaA |
| | | GGTFSSYVMGWFRRAPGKERDFVAA | |
| | | ISWSGGVTQYADSVKGRFTISRDNA | |
| | | KNTVYLQMNSLKPEDTAVYYCAADR | |
| | | TAVVPAQIRSYNYWGQGTQVTVSS | |
| 921 | NBX22086 | QVQLQESGGGLVQAGGSLRLSCAAS | CnaA |
| | | GRTFSNYIMGWFRQAPGKEREFVAA | |
| | | ISWSGGVTHYADSVKGRFTISRDNA | |
| | | NNTVFLQMNSLKPEDTAVYYCAAEV | |
| | | GYGSSWYYEADFGSWGQGTQVTVSS | |
| 922 | NBX22087 | QVQLQESGGGLVQAGGSLRLSCAAS | CnaA |
| | | GRTFSSYAMGWFRQAPGKEREFVAA | |
| | | ISWSGGITYYADSVKGRFTISRDNA | |
| | | KNTVYLQMNSLKPEDTAVYYCAADL | |
| | | EGAGNFREFGSWGQGTQVTVSS | |
| 923 | NBX22088 | QVQLQESGGGLVQAGGSLRLSCAAS | CnaA |
| | | GRTFSSYVMGWFRRAPGKEREFVAA | |
| | | ISWSGGVTQYADSVKGRFTASRDNA | |
| | | KNTVYLQMSSLKPEDTAVYYCAADR | |
| | | TAIVPAQIRSYDYWGQGTQVTVSS | |
| 924 | NBX22089 | QVQLQESGGGLEQPGGSLRLSCLVS | CnaA |
| | | GRTGGSFDMGWFRRTPGKEREFIAA | |
| | | ITWSGGSTEYADSVKGRFTISRDNG | |
| | | KNTVQLQMNSLKPEDTAVYYCAAGG | |
| | | FGALGVEHRYRYWGQGTQVTVSS | |
| 925 | NBX22090 | QVQLQESGGGLVQPGGSLRLSCAFS | CnaA |
| | | GNIFSISLMTWYRQAPGKQREFVAS | |
| | | ITSGGNTDYVNSGKGRFTISTDNAK | |
| | | NTMYLQLNSLOPEDTAVYYCHCDWC | |
| | | SWGQGTQVTVSS | |
| 926 | NBX22093 | QVQLQESGGGLVQAGGSLRLACVAS | CnaA |
| | | GRTFKIYGVAWFRQAPGKERDFVAG | |
| | | ISSSGGGTAYAGSVKDRFAISRDNA | |
| | | KNTVYLQMNSLKPEDTAVYYCAVGG | |
| | | WGRDDIYDYWGQGTQVTVSS | |
| 927 | NBX22094 | QVQLQQSGGGLVQAGGSLRLSCAAS | CnaA |
| | | ARTFSEYIMAWFRQAPGKEREFVAA | |
| | | ISSSGATTAYSNSVKDRFTISRDNA | |
| | | KNTLFLQLNNLKLEDTAAYYCAANR | |
| | | FLSAARYDRQRYDYWGQGTQVTVSS | |
| 928 | NBX22095 | QVQLQESGGGSVQAGGSLTLSCAVS | CnaA |
| | | GHTFSTYGMGWFRQAPGKEREYVAG | |
| | | ISASGGGTAYVSSVKARFTISRDNA | |
| | | KNTVYLQMNSLKPEDTAIYYCAAAR | |
| | | ASWGRQTTHDYWGQGTQVTVSS | |
| 929 | NBX22096 | QVQLQESGGGLVQPGGSLRLSCAAS | CnaA |
| | | GLTLNNYALRWYRQSAGSERELVAF | |
| | | ISAGGDIISYAESVKGRFTITRDNT | |
| | | KNTVYLQMNSLKPEDTAVYRCNRVS | |
| | | GDYWGQGTQVTVSS | |
| 930 | NBX22097 | QVQLQESGGGLVQAGGSLRLSCAAS | CnaA |
| | | GRTFSTYGMGWFRQAPGKEQEFVSS | |
| | | ISASGGGTAYANSVKARFTISRDNA | |
| | | KNTVYLQMNNLKPEDTAVYYCAAAR | |
| | | TGWGROTTYDYWGQGTQVTVSS | |
| 931 | NBX22098 | QVQLQESGGGLVQAGGSLRLSCAAS | CnaA |
| | | GGSLSNYIVGWFRQAPGKEREFVAI | |
| | | ISWSGEVTDYADSVKGRFTISRDNA | |
| | | GNTVNLQMNNLNPEDTAVYYCAGEH | |
| | | NGRSWYDVGNYAYWGQGTQVTVSS | |
| 932 | NBX22099 | QVQLQESGGGLVQAGGSLTLSCGPS | CnaA |
| | | GRTFSSGAMGWFRQAPGKEREFVAA | |
| | | ISRSGSRTLYADSVKGRFTITRDSA | |
| | | KPTVFLQMNSLKPEDTAVYYCAASL | |
| | | GRGYENSDSGAYDYWGQGTQVTVAS | |
| 933 | NBX22100 | QVQLQQSGGGLVQPGGSLRLSCAAS | CnaA |
| | | GFNLNYYAIGWFRQAPGKEREGVSC | |
| | | ISSSDGSTVYADSVKGRFTISRDNA | |
| | | KVYLQMNSLKPEDTAVYYCAARAGG | |
| | | EFYYCSGDSSADYWGQGTQVTVSS | |
| 934 | NBX22101 | QVQLQESGGGLVQAGGSLRLSCVPG | CnaA |
| | | RTFSSGAMGWFRQAPGKEREFVASI | |
| | | SRSSSRTLYADSVKGRFTITRDSAK | |
| | | NTMFLQMNSLKPEDTAVYYCAASLS | |
| | | RGYETSDSGAYDYWGQGTQVTVSS | |
| 935 | NBX22102 | QVQLQESGGGLVQAGGSLRLSCVAS | CnaA |
| | | GRTFRMYGVGWFRQGPGKERAFVAG | |
| | | ISSSGAGTAFAGSVEGRFAISRDNA | |
| | | KNTVYLQMNNLKPEDTAVYYCAGAD | |
| | | RDWGRDAIYDYWGQGTQVTVSS | |
| 936 | NBX22103 | QVQLQQSGGGLVQPGGSLRLSCAAS | CnaA |
| | | GFTWNNYGMRWYRQTPGKEREYVAF | |
| | | INSGGDIISYADSVKGRFTITRDNL | |
| | | RNVVYLQMNSLKPEDTAVYYCNRIS | |
| | | GDYWGQGTQVTVSS | |
| 937 | NBX22104 | QVQLQESGGGLVQAGGSLRLSCVPG | CnaA |
| | | RTFSFGAMAWFRQAPGKEREFVASI | |
| | | SRSVSRTLYADSVKGRFTITRDSAK | |
| | | NTMFLQMNSLKPEDTAVYYCAASQG | |
| | | RGYETSATGAYDYWGQGTQVTVSS | |
| 938 | NBX22105 | QVQLQQSGGGLVQAGGSLRLSCAAS | CnaA |
| | | GRTFSDYIMGWFRQAPGKEREFVAA | |
| | | ISWSGGVTHYADSVKGRFTISRDNA | |
| | | KNTVYLQMNSLKPEDTAVYYCAADG | |
| | | RSDSGGSYYPADFGSWGQGTQVTVS | |
| | | S | |
| 939 | NBX22106 | QVQLQESGGDLVQAGGSLRLSCAAS | CnaA |
| | | GFTFDDYAIGWFRQAPGKEREGVSC | |
| | | IERSGSSTWYADSVKGRFTISSDNA | |
| | | KNTVYLQMNSLKPEDMAVYYCAAGR | |
| | | VCSGYGGRIQGYEYDYWGQGTQVTV | |
| | | SS | |
| 940 | NBX22107 | QVQLQQSGGGLVQAGGSLRLSCVPG | CnaA |
| | | RTFSFGAMGWFRQAPGKEREFVASI | |
| | | SRSVSRTLYADSVKGRFTITRDSAK | |
| | | NTMFLQMNSLKPEDTAVYYCAASQG | |
| | | RGYETSDTGAYDYWGQGTQVTVTS | |
| 941 | NBX22108 | QVQLQESGGGLVQAGGSLRLSCAAS | CnaA |
| | | GRTFTMYGVGWFRQAPGKEREFVAG | |
| | | ISSSGGGTSYAGSVKGRFAISKDNA | |
| | | KNTVYLQMNSLKPEDTAVYYCAVGG | |
| | | WGRDDIYEYWGQGTQVTVSS | |
| 942 | NBX22109 | QVQLQESGGGLVQAGGSLTLSCGPS | CnaA |
| | | GRTFSSGAMGWFRQAPGKEREFVAA | |
| | | ISRSGSTTLYADSVKGRFTITRDSA | |
| | | KPTVFLQMNSLKPEDTAVYYCAASL | |
| | | GRGYENSDSGAYDYWGQGTQVTVAS | |
| 943 | NBX22110 | QVQLQESGGGLVQAGGSLRLSCVPG | CnaA |
| | | RTFSSGAMGWFRQAPGKEREFVASI | |
| | | SRSSSRTLYADSVKGRFTITRDSAK | |
| | | NTMFLQMNSLKPEDTAVYYCAASLS | |
| | | RAYETSDSGAYDYWGQGTQVTVSS | |
| 944 | NBX22111 | QVQLQQSGGGSVQAGGSLRLSCVAS | CnaA |
| | | GRTFSTYGMGWFRQAPGKEREYVAG | |
| | | ISASGGGTAYVSSVKARFTISRDNA | |
| | | KNTVYLQMNSLKPEDTAIYYCAAAR | |
| | | TSWGRQTTHDYWGQGTQVTVSS | |
| 945 | NBX22112 | QVQLQESGGGVVQAGGSLRLSCAAS | CnaA |
| | | GRTFSDYVMGWFRQAPGKEREFVAA | |
| | | ISWSGGVTHYADSVKGRFTISRDNA | |
| | | QNTAYLQMDSLKPEDTAVYYCAADA | |
| | | RSDSGRWYYPADFGSWGQGTQVTVS | |
| | | S | |
| 946 | NBX22119 | QVQLQESGGGLVQAGGSLRLSCAAS | CnaA |
| | | GRTFSSYAMGWFRQAPGKEREFVAA | |
| | | ISWSGGITWYADSVKGRFTISRDNA | |
| | | KNTVYLQMNSLKPEDTAVYYCAADT | |
| | | GADSGSYYYPADFGSWGQGTQVTVS | |
| | | S | |
| 947 | NBX22120 | QVQLQESGGGLVQAGGSLRLSCAAS | CnaA |
| | | GGTFSSYIMGWFRQGPGKEREFVAA | |
| | | ISWSGGVTYYADSVKGRFTISRDNA | |
| | | KNTVYLQMNSLKPEDTAVYYCAADA | |
| | | RSDSGRYYYPADFGSWGQGTQVTVS | |
| | | S | |
| 948 | NBX22121 | QVQLQQSGGGLVQAGGSLRLSCAAS | CnaA |
| | | GRTLSNYAMAWFRQAPGKERALVAS | |
| | | ISGSGSNTNYPNSVKDRFTISRDNA | |
| | | KNTVYLQMNSLKPEDTAVYYCAARE | |
| | | GLALRYATYDYWGQGTQVTVSS | |
| 949 | NBX22122 | QVQLQESGGGLVQPGGSLRLSCAFS | CnaA |
| | | GNIFSIALMTWYRQAPGKQREFVAS | |
| | | ITSGGNTNYVNSGKGRFTISTDNAK | |
| | | NTMYLQLNSLOPEDTAVYYCHCDWC | |
| | | SWGQGTQVTVSS | |
| 950 | NBX22123 | QVQLQQSGGGSVQPGGSLRLSCAAS | CnaA |
| | | GRTFSTYGMGWFRQAPGKEREFVAG | |
| | | ISASGGGTAYIGSVKARFTISRDNA | |
| | | KNTVYLQMNSLKPEDTAIYYCAVAR | |
| | | TGWGREATHDYWGQGTQVTVSS | |
| 951 | NBX22124 | QVQLQQSGGRLVQAGGSLRLSCAAS | CnaA |
| | | GRTFSDYEMAWFRQGPGKEREFVAG | |
| | | ISGSGGTTAFANFVKGRFIISRDNA | |
| | | KNTMYLQMNNLKLEDTGVYYCAARV | |
| | | PRVGRFDENEYEYWGQGTQVTVSS | |
| 952 | NBX22125 | QVQLQQSGGGLVQAGGSLRLSCAAS | CnaA |
| | | GDTFSAYGMGWFRQTPGKEREFVAG | |
| | | ISSGGGGTAYAGSVKDRFTIFRDNA | |
| | | KNTVYLQMNSLKPEDTAVYYCARAV | |
| | | RSWGRAAEHDYWGQGTQVTVSS | |
| 953 | NBX22126 | QVQLQESGGGLVQAGVSLTLSCAAS | CnaA |
| | | GRTASTSTMAWFRQPPGKEREFVGV | |
| | | WSGGSTNYVNDRLIISRDNAKNTVY | |
| | | LQMNSLKPEDTAVYYCAASPRIWYR | |
| | | DTNYKRATWYDYWGQGTQVTVSS | |
| 954 | NBX22127 | QVQLQESGGGLVQAGGSLRLSCAAS | CnaA |
| | | GRTFSDYAMAWFRQVPGKEREFVAS | |
| | | ISWSGGVTWYADSVKGRFTISRDNA | |
| | | KNTVYLQMNSLKPEDTAVYYCAADA | |
| | | RSDHASYYYPADFGFWGQGTQVTVS | |
| | | S | |
| 955 | NBX22128 | QVQLQQSGGGLVQAGGSLRLSCAAS | CnaA |
| | | GRTFSGYIMAWFRQAPGKEREFVAA | |
| | | ISSSGATTAYSASVKDRFTISRDNA | |
| | | KNTSFLQMNNLKLEDTAAYYCAADQ | |
| | | FDSTARYDRRQYEYWGQGTQVTVSS | |
| 956 | NBX25006 | QVQLQQSGGGLVQAGGSLRLSCTAS | NetB |
| | | GSTLSTYAFNWFRQAPGKQRELVAT | |
| | | ISRGGKTTYADSVKGRFTVSRDNAK | |
| | | STVYLQMNSLKPEDTAVYYCDTVGY | |
| | | YPEIRSGQGTQVTVSS | |
| 957 | NBX25007 | QVQLQQSGGGLVQPGGSLRLSCAAS | NetB |
| | | GSIFGYAMGWYRQAPGKQREWVAEI | |
| | | SRGGTPNYADSVKGRFTISRDNAKN | |
| | | TVYLQMNSLKADDTAVYYCRCLGFD | |
| | | YWGQGTQVTVSS | |
| 958 | NBX25008 | QVQLQESGGGLVQAGGSLTLSCTAS | NetB |
| | | GSIFGIKAMGWYRQAPGKERDLVAQ | |
| | | ITEGGTTNYADSVKGRFTISRDTAK | |
| | | NTLYLQMNSLKPEDTAVYYCNSLWS | |
| | | LLEQYPRYFWGQGTQVTVSS | |
| 959 | NBX25009 | QVQLQESGGGLVQPGGSLRLSCAAS | NetB |
| | | GFTPVYYAISWFRQAPGKEREWVSC | |
| | | ISSVDGKTNYADSVKGRFTISRMNA | |
| | | KNTVYLQMNTLKPDDTAVYYCAAEG | |
| | | PPYDPGQLCPYNDMDYWGKGTLVTV | |
| | | SS | |
| 960 | NBX25010 | QVQLQESGGGSVRSGGSLTLSCTAS | NetB |
| | | LNSGSIAGMGWYRQAPGKERELVAG | |
| | | ITRGGSSNYGDSVKGRFTVSRDNAK | |
| | | NAMYLQMTSLKPEDTAVYRCFAYRK | |
| | | DDFGFEVLYWGQGTQVTVSS | |
| 961 | NBX25011 | QVQLQESGGGLVQAGGSLTLSCAAT | NetB |
| | | GSTFSNYAMNWYRQAPGKQRELVAT | |
| | | ISRGGVATYADSVKGRFTVSRDNAK | |
| | | NTVYLQANSLKPEDTALYYCDTVGY | |
| | | YSEVRQGQGTQVTVSS | |
| 962 | NBX25012 | QVQLQESGGGLVQAGGSLRLSCTAS | NetB |
| | | GSTLSTYAFNWFRQAPGKQRELVAT | |
| | | ISRGGRTTYAESAKGRFTVSRDNAK | |
| | | STVYLQMNSLKPEDTAVYYCDTVGY | |
| | | YPEIRSGQGTQVTVSS | |
| 963 | NBX25013 | QVQLQQSGGGSVQAGGSLRLSCAGS | NetB |
| | | GITFSAYAMGWYRQAPGKQRELVAD | |
| | | ISRAGITTYGDSVKGRFIISRDNEK | |
| | | NTVYLQMNSLKPEDTAVYYCRTLGF | |
| | | AYWGQGTQVTVSS | |
| 964 | NBX25014 | QVQLQESGGGSVQAGGSLRLSCAGS | NetB |
| | | GITFSAYAMGWYRQAPGKQRELVAD | |
| | | ISRAGITTYGDSVKGRFIISRDNEK | |
| | | NTVYLQMNSLKPEDTAVYYCRTLGF | |
| | | PYWGQGTQVTVSS | |
| 965 | NBX25015 | QVQLQESGGGLVQPGESLRLSCVAS | NetB |
| | | GSIAPYAMGWYRQAPGQQREWVAHI | |
| | | SRGSLPDYADSVKGRFFISRDNAKN | |
| | | TVYLQMNSLKPEDTAVYQCRTLGFD | |
| | | YWGQGTQVTVSA | |
| 966 | NBX25016 | QVQLQQSGGGLVQAGGSLRLSCAAS | NetB |
| | | GSTFSSYVMGWYRQAPGKQRELVAG | |
| | | ISSGGSTNYADSVKGRFTISRDNVK | |
| | | NTVSLQMNSLKPEDTAVYYCNLRDR | |
| | | FGHGYWGQGTQVTVSS | |
| 967 | NBX25017 | QVQLQQSGGGLVQPGGSLRLSCAGS | NetB |
| | | GTTLSNYAMGWYRQAPGKQRESVAD | |
| | | ISRGGTPDYADSVKGRFTISRDNAK | |
| | | NTVTLQMNRLEPEDTAVYYCKILGF | |
| | | DVWGQGTQVTVSS | |
| 968 | NBX25018 | QVQLQESGGGSVRAGGSLRLSCTAS | NetB |
| | | LNSGSIAGMGWYRQAPGKERELVAG | |
| | | ITRGGSSNYGDSVKGRFTVSRDNAK | |
| | | NTLYLQMSSLKPEDTAVYYCFAYRK | |
| | | DDFGFEVLYWGQGTQVTVSS | |
| 1791 | NBX0572 | QVQLQQSGGGSVQAGGSLRLSCAAS | CnaA |
| | | RPTISGYAMGWFRQAPGKEREFVAA | |
| | | ISGSGASTGYSNSVKGRFTVFRDNT | |
| | | QDTVYLQMNSLKPEDTAIYFCAVYS | |
| | | APDPTFDHYHMYSANYDNWGQGTQV | |
| | | TVSS | |
| 1792 | NBX0573 | QVQLQESGGGLVQAGTTLRLSCAVS | CnaA |
| | | GPTISSYAVGWFRQAPGKEREFVGV | |
| | | SWSASSTVYADSMKGRFAISRDNAK | |
| | | NTVSLQMNSLRPEDTAIYYCASMRA | |
| | | SYCAGYRCHQRAQTYDYWGQGTQVT | |
| | | VSS | |
| 1793 | NBX0575 | QVQLQESGGGLVQAGDSLRVSCLAS | CnaA |
| | | RRTLSSYVMGWFRQAPGKGREFVGG | |
| | | ISKDGRSTRYANFVEGRFTVSRDNA | |
| | | KNTVYLQMNSLKPEDTATYYCAASD | |
| | | YNGGGIPSLRLDYNYWGQGTQVTVS | |
| | | S | |
| 1794 | NBX0576 | QVQLQQSGGGLVQAGGSLRLSCAAT | CnaA |
| | | GSTNTFNKMDWYRQAPAKQRELVAT | |
| | | PMSRGDPYYADSVKGRFTISGDNAK | |
| | | NAVYLQMNNLKPEDTAVYYCRGRIG | |
| | | ERVYWGQGTQVTVSS | |
| 1795 | NBX0577 | QVQLQESGGGMVQAGGSLRLSCVAS | CnaA |
| | | GRAIGELSMGWYRQAPGKQRELVAR | |
| | | AIWSGGTVYGDSVRGRFTISRDSAK | |
| | | NTVYLQMNDLKPEDTALYYCNVGAN | |
| | | YWGQGTQVTVSS | |

TABLE 2
Unique SEQ IDs for V_{H}H CDRs of this disclosure

| | CDR1 Amino | CDR1 | CDR2 Amino | CDR2 | CDR3 Amino | CDR3 | |
|---|---|---|---|---|---|---|---|
| | Acid | SEQ ID | Acid | SEQ ID | Acid | SEQ ID | |
| NBX | Sequence | NO: | Sequence | NO: | Sequence | NO: | Antigen |
| NBX0301 | QRASSLFAM | 57 | ISWNGDKS | 107 | AAHRASFELGF | 157 | NetB |
| | | | | | ATHDYDF | | |
| NBX0302 | GSIFSISSA | 58 | IFSDGST | 108 | AVDGY | 158 | NetB |
| NBX0303 | GRTLSYWTM | 59 | INWSSGT | 109 | AAHRASFGLGY | 159 | NetB |
| | | | | | QTHEYDF | | |
| NBX0304 | GGTFSSYTM | 60 | ITWNSEVT | 110 | AAGRAGSGFTS | 160 | NetB |
| NBX0305 | GFTLDKYAV | 61 | ISSIDDST | 111 | MTIPLPYGSTCD | 161 | NetB |
| | | | | | IPSRSDLLAINY | | |
| NBX0306 | GFTVPYYYI | 62 | IASSSGKA | 112 | AALRKYGSTCYL | 162 | NetB |
| | | | | | HVLEYDY | | |
| NBX0309 | GSTFNNYMI | 63 | ISGSGAGT | 113 | ARRMSRSGIFGL | 163 | NetB |
| | | | | | RDYDS | | |
| NBX0311 | GRTFSNADM | 64 | ISWSGGRT | 114 | AAGGYSNLPTS | 164 | NetB |
| | | | | | YGY | | |
| NBX0316 | GRAFSTYGM | 65 | ISSSGAGS | 115 | AASTTSWGKFA | 165 | CnaA |
| | | | | | HYIY | | |
| NBX0317 | GGTFSSYIM | 66 | ISWSGGVT | 116 | AADSRISAGGSY | 166 | CnaA |
| | | | | | YEADFGS | | |
| NBX0318 | GSIMSIRVM | 67 | MSRGNTI | 117 | AALLDSYY | 167 | NetB |
| NBX0319 | ASIISIRVM | 68 | MSRGGTI | 118 | TALLDSYY | 168 | NetB |
| NBX0320 | GSIFSIRVM | 69 | MSRGGTI | 119 | AALLDSYY | 169 | NetB |
| NBX0321 | GFTLDKYAV | 70 | ISSIDDST | 120 | MTIPLPYGSTCR | 170 | NetB |
| | | | | | IPSRSDLLAINY | | |
| NBX0322 | GRTFSTYAM | 71 | ITRGGNT | 121 | AADRIIVPRDP | 171 | NetB |
| | | | | | MDY | | |
| NBX0326 | GRTFSAIHM | 72 | ISWSGGGT | 122 | AASDTDWGRS | 172 | CnaA |
| | | | | | ASYDY | | |
| NBX0327 | GGTFSSYVM | 73 | ISWSGGVT | 123 | AADSRISAGGSY | 173 | CnaA |
| | | | | | YEADFGS | | |
| NBX0328 | GRTFSSYTM | 74 | ISWSGT | 124 | AVGSRRLYYSSD | 174 | Cpa |
| | | | | | INY | | |
| NBX0329 | GLTVSRYTM | 75 | ISWSGT | 125 | AAGSRRLYYSN | 175 | Cpa |
| | | | | | DINY | | |
| NBX0330 | SRTFSNYAM | 76 | INGDTTFT | 126 | AARQWNPTMR | 176 | Cpa |
| | | | | | ERDYGY | | |
| NBX0331 | GRVFENYFM | 77 | TNWNTATN | 127 | AATGSRTYDVV | 177 | Cpb2 |
| | | | WNT | | DYYDY | | |
| NBX0332 | GRTFSSYSM | 78 | ITYSGITT | 128 | AASYSASRSYPF | 178 | Cpb2 |
| | | | | | GEYDY | | |
| NBX0333 | GRTFSSYSM | 79 | ITYSGIST | 129 | AASYSASRSYPF | 179 | Cpb2 |
| | | | | | GEYDY | | |
| NBX0334 | GFTFSNSAM | 80 | INIGGDSR | 130 | AKGLASTI | 180 | Cpb2 |
| NBX0335 | GFTFSNSAM | 81 | IEVGGGR | 131 | SKGLASTI | 181 | Cpb2 |
| NBX0336 | GFTFSNSAM | 82 | IGIDGGR | 132 | AKGLASTI | 182 | Cpb2 |
| NBX0337 | GFTFSNSAM | 83 | IGIGGGTT | 133 | AKGLASTI | 183 | Cpb2 |
| NBX0338 | GRSFSSYTM | 84 | ISWSGT | 134 | AVGSRRLYYSSD | 184 | Cpa |
| | | | | | INY | | |
| NBX0339 | GLTVSRYTM | 85 | ISWSGT | 135 | AAGSRRLHYSS | 185 | Cpa |
| | | | | | DINY | | |
| NBX0340 | GRTFSTSTL | 86 | IRYTSDYTART | 136 | AAAKYGMGYS | 186 | Cpa |
| | | | T | | DPSGYTY | | |
| NBX0341 | SRTFSNYAM | 87 | ITGDTAFT | 137 | AARQWNPTMR | 187 | Cpa |
| | | | | | ERDYGY | | |
| NBX0342 | GRRFRLYHM | 88 | ISWSGGTT | 138 | AVDRLIESFSDP | 188 | Cpb2 |
| | | | | | TAWPRM | | |
| NBX0343 | GFTFSNSAM | 89 | INIGGGTT | 139 | AKGLASTI | 189 | Cpb2 |
| NBX0344 | GFTFSNSAM | 90 | INIGGGTR | 140 | AKGLASTI | 190 | Cpb2 |
| NBX0345 | GRKFRLYHM | 91 | ISWSGGST | 141 | AVDRLIESFSDP | 191 | Cpb2 |
| | | | | | TAWPRM | | |
| NBX0346 | GFTFSNSAM | 92 | INIGGGT | 142 | AKGLASTI | 192 | Cpb2 |
| NBX0347 | GRTFSSYDM | 93 | ISYNI | 143 | AAVQRRGSYSY | 193 | Cpb2 |
| | | | | | DRAQSYDY | | |
| NBX0348 | GFTFSNSAM | 94 | IEIGGTR | 144 | AKGLASTI | 194 | Cpb2 |
| NBX0349 | GFTFSNSPM | 95 | INIGAGTT | 145 | AKGLASTI | 195 | Cpb2 |
| NBX0350 | GFTFSNSAM | 96 | INIGGGDK | 146 | AKGLASTI | 196 | Cpb2 |
| NBX0351 | GFTFSNSAM | 97 | IETGGTK | 147 | AKGLASTI | 197 | Cpb2 |
| NBX0352 | GFTFSNSPM | 98 | INIGEGTT | 148 | AKGLASTI | 198 | Cpb2 |
| NBX0353 | GFTFSNSPM | 99 | INIGGDTR | 149 | AKGLASTI | 199 | Cpb2 |
| NBX0354 | GFTFSNSAM | 100 | VNIDGGR | 150 | AKGLASTI | 200 | Cpb2 |
| NBX0355 | GFTFSNSAM | 101 | ISIDGGR | 151 | AKGLASTI | 201 | Cpb2 |
| NBX0356 | GGKFTLYHM | 102 | ISWSGRST | 152 | AVDRLIEKFSDP | 202 | Cpb2 |
| | | | | | TAWPRMDS | | |
| NBX0357 | GRTASM | 103 | ITRSSIYT | 153 | AADSTMSGSSR | 203 | Cpb2 |
| | | | | | YSSDYAY | | |
| NBX0358 | GFTFSNSPM | 104 | IDIGGNR | 154 | AKGLASTI | 20 | Cpb2 |
| NBX0359 | GRRFTLYHM | 105 | ISWSGGST | 155 | AVDRLIESFSDP | 205 | Cpb2 |
| | | | | | TAWPRMDY | | |
| NBX0360 | GRRFSLYHM | 106 | ISWSGGTT | 156 | AVDRLIESFSDP | 206 | Cpb2 |
| | | | | | TAWPRMDY | | |
| NBX0363 | ASIISIRVM | 341 | MSRGGTI | 459 | AALLDSYY | 577 | NetB |
| NBX0364 | GSIMSIRVM | 342 | MSRGGTI | 460 | TALLDSYY | 578 | NetB |
| NBX0365 | GSIFSIRVM | 343 | MSRGGTI | 461 | TALLDSYY | 579 | NetB |
| NBX0369 | ASIFSIRVM | 344 | MSRGNTI | 462 | AALLDSYY | 580 | NetB |
| NBX0370 | GSIMSIRVM | 345 | MSRGGTI | 463 | AALLDSYY | 581 | NetB |
| NBX0373 | ASIMSIRVM | 346 | MSRGNTI | 464 | AALLDSYY | 582 | NetB |
| NBX0374 | ASIMSIRVM | 347 | MSRGGTI | 465 | AALLDSYY | 583 | NetB |
| NBX0379 | GSIMSIRVM | 348 | MSRGNTI | 466 | AALLDSYY | 584 | NetB |
| NBX0380 | GSIISIRVM | 349 | MSRGGTI | 467 | AALLDSYY | 585 | NetB |
| NBX0381 | ASIISIRVM | 350 | MSRGNTI | 468 | AALLDSYY | 586 | NetB |
| NBX0501 | GSIFSINVM | 351 | ITSGGST | 469 | NAAQSRTSWLF | 587 | NetB |
| | | | | | PDEYDY | | |
| NBX0502 | GRTFSIYAM | 352 | INRGGGTT | 470 | AADRVTDTYYYL | 588 | NetB |
| | | | | | NPESYDY | | |
| NBX0503 | GSGRRVGYM | 353 | ISRAGAT | 471 | FASLIDAGTY | 589 | NetB |
| NBX0504 | GRTFSIYAM | 354 | INRSGGTT | 472 | AADRVTDTYYYL | 590 | NetB |
| | | | | | NPESYDY | | |
| NBX0505 | GMSFSLGTI | 355 | ITNADTT | 473 | NTATS | 591 | NetB |
| NBX0506 | GSGRRVGYM | 356 | ISRAGAT | 474 | FASVFDAGTY | 592 | NetB |
| NBX0507 | GSGRRVGYM | 357 | ISRAGAT | 475 | FASIFDAGTY | 593 | NetB |
| NBX0508 | GSGSRINYM | 358 | INRTGAA | 476 | FASYLGAGAY | 594 | NetB |
| NBX0509 | GRTFSTYTV | 359 | ITWNGGTI | 477 | VMGAAGQGW | 595 | NetB |
| | | | | | RY | | |
| NBX0510 | GSGSRINYM | 360 | INRTGAA | 478 | WASYLGAGTY | 596 | NetB |
| NBX0511 | GFTFSRNYM | 361 | IYSDDST | 479 | SKEGGL | 597 | NetB |
| NBX0512 | GSGRRVGYM | 362 | ISRAGAT | 480 | FASVFDAGTY | 598 | NetB |
| NBX0513 | GSGRRVGYM | 363 | ISRAGAT | 481 | FASLFDAGTY | 599 | NetB |
| NBX0514 | GRTFSGRTM | 364 | ITWSGGTT | 482 | ASDGPWRATTP | 600 | CnaA |
| | | | | | DAYDY | | |
| NBX0515 | GSIGTIDSM | 365 | IMFSGRT | 483 | YSNQY | 601 | CnaA |
| NBX0517 | EFSLLFGTI | 366 | VSSSDGST | 484 | ATRCTVVPGIT | 602 | CnaA |
| NBX0518 | GSITRVGGM | 367 | INEVGNT | 485 | WIPPIP | 603 | CnaA |
| NBX0519 | PFSLRLGVV | 368 | ISSSEGST | 486 | ATRCTVVPGIT | 604 | CnaA |
| NBX0520 | ARTSSSRAM | 369 | ISWSGGRT | 487 | AARRSDFTGDY | 605 | CnaA |
| | | | | | AYSGRSAYDY | | |
| NBX0521 | GSTFIFDKM | 370 | LMSRGDP | 488 | RGRAGERVY | 606 | CnaA |
| NBX0522 | GRTFSGVIV | 371 | TLWSGGST | 489 | AAKYGGSLSYM | 607 | CnaA |
| | | | | | HPTGYTY | | |
| NBX0523 | RIVFTISTM | 372 | INRSGALT | 490 | AASKANMPALP | 608 | CnaA |
| | | | | | ANYDY | | |
| NBX0524 | GSITRLGSM | 373 | ITAVGNT | 491 | WIPPIP | 609 | CnaA |
| NBX0525 | GSITRLGGM | 374 | IDTVGNT | 492 | WIPPIP | 610 | CnaA |
| NBX0526 | ERAFMYNM | 375 | RNWNVERT | 493 | ATTRVWPTQH | 611 | CnaA |
| | | | | | QMGQIEY | | |
| NBX0527 | SSFNTM | 376 | ITSGGTI | 494 | VADWQYGSTW | 612 | CnaA |
| | | | | | NY | | |
| NBX0528 | GRNFDYYSM | 377 | INWRGAVI | 495 | AAASSSSRLLEPI | 613 | CnaA |
| | | | | | GYNY | | |
| NBX0529 | GSMFSINDM | 378 | ISSGGTT | 496 | AGNLKRSETSYY | 614 | CnaA |
| | | | | | WK | | |
| NBX0530 | GRTFSRYHM | 379 | ISLSGGGT | 497 | TADRHEWGRL | 615 | CnaA |
| | | | | | MKGDY | | |
| NBX0531 | GRTSNSYNM | 380 | ISWTGGFT | 498 | AATSRSLTSAM | 616 | CnaA |
| | | | | | TREIRAYDY | | |
| NBX0532 | GSTFSFNKM | 381 | FMNDGNT | 499 | RGRAGMEVY | 617 | CnaA |
| NBX0533 | PLTLRLGPI | 382 | ISSRDDK | 500 | ATRCTVVPGIS | 618 | CnaA |
| NBX0534 | GRNFGYYTM | 383 | ITWRGVI | 501 | AAASSSSRPLEPI | 619 | CnaA |
| | | | | | GYNY | | |
| NBX0535 | GDIFSAAGM | 384 | VTWDGGTT | 502 | AAGNTGPFNLL | 620 | CnaA |
| | | | | | HSSAQYAY | | |
| NBX0536 | PLTLRLGAI | 385 | ITSTEDK | 503 | ATRCTVVPGIS | 621 | CnaA |
| NBX0537 | GSITRIGGM | 386 | INTVGNT | 504 | WIPPLP | 622 | CnaA |
| NBX0538 | GRSFSRYIM | 387 | IAPSGGSA | 505 | AARYDMDYEYK | 623 | NetB |
| | | | | | T | | |
| NBX0539 | GSGSRIGFM | 388 | INRTGAT | 506 | FASVVDAGTY | 624 | NetB |
| NBX0540 | GLTFSDYAM | 389 | ISLTAAST | 507 | AAQGRILRGRG | 625 | NetB |
| | | | | | LFKASDYDY | | |
| NBX0541 | GTISIFDPM | 390 | ISEGST | 508 | RLSRYYNSNIY | 626 | NetB |
| NBX0542 | RNIYGINVI | 391 | SANGGTT | 509 | KAELYTLQHNYE | 627 | NetB |
| | | | | | Y | | |
| NBX0543 | GTLSLFDPM | 392 | ISGLST | 510 | HLSRYYNSNIY | 628 | NetB |
| NBX0544 | GRVLSINAM | 393 | ITNGGST | 511 | LAEERPYYGGPL | 629 | NetB |
| | | | | | EY | | |
| NBX0545 | RTTFRVGTM | 394 | ITSGGST | 512 | FANIVDRPVS | 630 | NetB |
| NBX0546 | GSGSRIGLM | 395 | IKGTGTT | 513 | FASVLGAGTY | 631 | NetB |
| NBX0547 | GTISLFDSM | 396 | ITEGST | 514 | RLSRYYNSNIY | 632 | NetB |
| NBX0548 | ETSLNFDDM | 397 | INTFPAGTTA | 515 | NAGDY | 633 | NetB |
| NBX0549 | GSDSSINYM | 398 | ISRDGRS | 516 | YVDPLGRVPR | 634 | NetB |
| NBX0550 | GTVNLM | 399 | IKGTGTT | 517 | FASVLGAGTY | 635 | NetB |
| NBX0551 | GSIFSRNII | 400 | INTGGRT | 518 | NAPSLGY | 636 | NetB |
| NBX0552 | GSGSINYM | 401 | INRTGAA | 519 | FASALGAGVY | 637 | NetB |
| NBX0553 | GSGWRVGYM | 402 | ISRAGAT | 520 | FASIIDAGTY | 638 | NetB |
| NBX0561 | GENFSTYVM | 403 | HNWRGGGT | 521 | AARSGGSYTYT | 639 | CnaA |
| | | | | | GSYHY | | |
| NBX0801 | GRTFSSYAM | 404 | ISRSGGST | 522 | AANRYGSSSYQ | 640 | CnaA |
| | | | | | GQYAS | | |
| NBX0802 | GRTFSSYHM | 405 | ISRSGGFT | 523 | AAQQWPDPRN | 641 | CnaA |
| | | | | | PNGYDY | | |
| NBX0803 | GRTFINYGM | 406 | VSISGAGT | 524 | AAAKAGHWGR | 642 | CnaA |
| | | | | | DANYDY | | |
| NBX0804 | GRTLTAYGM | 407 | VSLSGAST | 525 | AAAKAGQWGR | 643 | CnaA |
| | | | | | DAKYDY | | |
| NBX0805 | GRTFSTYAM | 408 | ISWSGGRI | 526 | TADLKGLWALG | 644 | CnaA |
| | | | | | LPGHYASWDS | | |
| NBX0806 | GSIGSINIM | 409 | FTSGGST | 527 | RARRGWAIY | 645 | CnaA |
| NBX0807 | GRTFSSYGM | 410 | ISRTGSGT | 528 | AADSGGSWGR | 646 | CnaA |
| | | | | | GTTYDY | | |
| NBX0808 | ARASSIGAM | 411 | VTAGADTT | 529 | AAYNTAGWGE | 647 | CnaA |
| | | | | | PHQSYRY | | |
| NBX0809 | GLTFGNYDM | 412 | ISSSGAYT | 530 | AGRRSVVVRSF | 648 | CnaA |
| | | | | | DYDY | | |
| NBX0810 | GRIFNANGM | 413 | LYRSGST | 531 | NVNWALHDS | 649 | CnaA |
| NBX0811 | ERTFSSDGM | 414 | ISRTGSAT | 532 | AANSGGHWW | 650 | CnaA |
| | | | | | RGATYDY | | |
| NBX0812 | GTIFSANGM | 415 | LYRDGST | 533 | NVNWALHDS | 651 | CnaA |
| NBX0847 | TRASIVGAM | 416 | IAAGSPSTP | 534 | AAYNTANWGQ | 652 | CnaA |
| | | | | | PHQSYRH | | |
| NBX0866 | GSILNINVM | 417 | IYRDGST | 535 | NVVTYGSNRRD | 653 | CnaA |
| | | | | | F | | |
| NBX0867 | GRTFSSYAM | 418 | ISRSGGST | 536 | AANRYGSSSYQ | 654 | CnaA |
| | | | | | GQYGS | | |
| NBX0868 | GRTFSSYAM | 419 | ISRSGGST | 537 | AANRYGSSSYQ | 655 | CnaA |
| | | | | | GQYDY | | |
| NBX0869 | GTIFSINGM | 420 | LYRGGST | 538 | NVNWALQDS | 656 | CnaA |
| NBX0870 | TSDGSINVM | 421 | ITSLGSQ | 539 | RARRGWAIY | 657 | CnaA |
| NBX0871 | GRTFNIYAM | 422 | ISDSGGSA | 540 | AADLTGLWALG | 658 | CnaA |
| | | | | | LPGHYASWDS | | |
| NBX0872 | GFTFRSSAM | 423 | IGSDGENI | 541 | QLGRTVLDYF | 659 | CnaA |
| NBX0873 | GRTFINYGM | 424 | VSSSGAGT | 542 | AAAKAGQWGR | 660 | CnaA |
| | | | | | YANYDY | | |
| NBX0874 | GRTFSSYAM | 425 | ISRSGGTT | 543 | AANPYGSSSYQ | 661 | CnaA |
| | | | | | GQYGS | | |
| NBX0875 | GRAFSGYAM | 426 | ISRGGGTT | 544 | AANRYGSSSYQ | 662 | CnaA |
| | | | | | GQYGS | | |
| NBX0876 | GRTFINYGM | 427 | VSSSGAGT | 545 | AAAKAGHWGR | 663 | CnaA |
| | | | | | DANYDY | | |
| NBX0877 | GSISSITFM | 428 | IARSGTT | 546 | YVDRRGAVPT | 664 | NetB |
| NBX0878 | GSISSITFM | 429 | IARSGTT | 547 | YVDRRDVVPT | 665 | NetB |
| NBX0879 | GTGFPIITFM | 430 | ISRGGVA | 548 | YADRFSGSPT | 666 | NetB |
| NBX0880 | VSSIGTM | 431 | ISRVGTT | 549 | FANVISGPVY | 667 | NetB |
| NBX0881 | TRFFSNYAM | 432 | ISRDGAVP | 550 | AASRQGNPYAQ | 668 | NetB |
| | | | | | TSYDY | | |
| NBX0883 | GSADSIKIM | 433 | ITSGGTT | 551 | NALVSRRDSAA | 669 | NetB |
| | | | | | YFA | | |
| NBX0884 | ESIVSITPM | 434 | TTRDGAP | 552 | KARKDSHDY | 670 | NetB |
| NBX0885 | ETIGSIQRM | 435 | RTNGGTT | 553 | NAHIREYYSTYD | 671 | NetB |
| | | | | | Y | | |
| NBX0886 | GSISRIRDI | 436 | ISSGGST | 554 | NALFNPIDGPA | 672 | NetB |
| | | | | | RYY | | |
| NBX0887 | GSISRIYDM | 437 | ISRGGST | 555 | TALFNPVDGTA | 673 | NetB |
| | | | | | RYY | | |
| NBX0888 | GTIFSINVM | 438 | ITSGGQI | 556 | NAASSTWPPRD | 674 | NetB |
| | | | | | YDY | | |
| NBX0889 | RSISSIAAM | 439 | ITNGGST | 557 | NADERPYYGDS | 675 | NetB |
| | | | | | VLS | | |
| NBX0890 | GTGFPIITFM | 440 | INRGGVA | 558 | YADRFSGSPT | 676 | NetB |
| NBX0891 | GRTFSNYHM | 441 | ISRGTSTT | 559 | AADADRSTTIYS | 677 | NetB |
| | | | | | RDIYDY | | |
| NBX0892 | EGTFSNYRM | 442 | ISRDGAVP | 560 | AASRQGLPYVE | 678 | NetB |
| | | | | | TSYDY | | |
| NBX0893 | GSISSITFM | 443 | SARRGTT | 561 | YVDRRDEVPT | 679 | NetB |
| NBX0894 | GGTFSSYVM | 444 | IRWSRGST | 562 | AADGNPAKLVL | 680 | NetB |
| | | | | | DQYGMDY | | |
| NBX0895 | GSISEITYM | 445 | IARVGTT | 563 | YVDQRGVVPT | 681 | NetB |
| NBX0896 | ARASSIGAM | 446 | VNAGADTT | 564 | AAYNTAGWGE | 682 | CnaA |
| | | | | | PHQSYRY | | |
| NBX0897 | GSIFIISTM | 447 | ITSGGST | 565 | NAEVHVWGVP | 683 | CnaA |
| | | | | | GPRDY | | |
| NBX0898 | GRTFSSYAM | 448 | ISRSGGST | 566 | AANPYGSSSYQ | 684 | CnaA |
| | | | | | GQYAS | | |
| NBX0899 | GSIFSSNGM | 449 | LYRSGST | 567 | NVNWALHDS | 685 | CnaA |
| NBX08100 | GRTFSAYGM | 450 | VSGGGGGT | 568 | AAATAGHWGR | 686 | CnaA |
| | | | | | DANYDY | | |
| NBX08101 | GSIFSSNGM | 451 | LFRSGST | 569 | NVNWALHDS | 687 | CnaA |
| NBX08102 | GRTFSSYAM | 452 | ISRSGGTT | 570 | AANPYGSSSYQ | 688 | CnaA |
| | | | | | GQYGS | | |
| NBX08103 | GIIHSINVM | 453 | ISSGGRT | 571 | TMVWGLRYY | 689 | CnaA |
| NBX08104 | TSIFSSNGM | 454 | LFRSGST | 572 | NVNWALHDS | 690 | CnaA |
| NBX08105 | GRTFSSYAM | 455 | ISRGGGTT | 573 | AANPYGSSSYQ | 691 | CnaA |
| | | | | | GQYGS | | |
| NBX08106 | GSIFSSNGM | 456 | LYRSGST | 574 | NVNWALHDS | 692 | CnaA |
| NBX08107 | GSIFSSNGM | 457 | LYRSGST | 575 | NVNWALHDS | 693 | CnaA |
| NBX08108 | RSILSANGM | 458 | LYRSGST | 576 | NVNWALHDS | 694 | CnaA |
| NBX03161 | GRSLSTVSM | 969 | IAWSGGRT | 1243 | AGYRGVLFITTK | 1517 | Cpa |
| | | | | | SAYDY | | |
| NBX03162 | GFTFSSYWM | 970 | ISTGGTST | 1244 | AKLVAYGMDY | 1518 | Cpa |
| NBX03163 | GRTFSRYTM | 971 | ISWSGT | 1245 | AVGSRRLYYSSD | 1519 | Cpa |
| | | | | | INY | | |
| NBX03164 | GRTFSSYTM | 972 | ISWSGT | 1246 | VVGSRRLYYSSD | 1520 | Cpa |
| | | | | | INY | | |
| NBX03165 | ERTFSRYTM | 973 | ISWSGT | 1247 | AQGSRRLYYSSD | 1521 | Cpa |
| | | | | | IDY | | |
| NBX03166 | GFIVNSYWM | 974 | ISTNAYST | 1248 | ARGGTAQEGG | 1522 | Cpa |
| | | | | | MDY | | |
| NBX03167 | GHDFSKYTM | 975 | ISWSGT | 1249 | AVGSRRLYYSSD | 1523 | Cpa |
| | | | | | IDY | | |
| NBX03168 | GFTFSRYTM | 976 | ISWSGT | 1250 | ASGSRRLYYSSD | 1524 | Cpa |
| | | | | | IDY | | |
| NBX03169 | GRTFSRYTM | 977 | ISWSGT | 1251 | ASGSRRLYYSSD | 1525 | Cpa |
| | | | | | IDY | | |
| NBX03170 | GRTFSRYTM | 978 | ISWSGT | 1252 | VVGSRRLYYSSD | 1526 | Cpa |
| | | | | | IDY | | |
| NBX03171 | GRDFSRYTM | 979 | ISWSGT | 1253 | AVGSRRLYYSSD | 1527 | Cpa |
| | | | | | IDY | | |
| NBX03172 | GTFSSYTM | 980 | ISWSGT | 1254 | AVGSRRLYYSSD | 1528 | Cpa |
| | | | | | INY | | |
| NBX03173 | GRTFSSYAM | 981 | LSGSGATT | 1255 | AASGHFGLRGT | 1529 | Cpa |
| | | | | | YNYEY | | |
| NBX03174 | GRTGIKWTM | 982 | ITWSGDT | 1256 | AADRIYNEDRYY | 1530 | Cpa |
| | | | | | Y | | |
| NBX03175 | GSIFRIIAM | 983 | ITNGGAT | 1257 | AADHFGRTPY | 1531 | Cpa |
| NBX03176 | GRTFSSYTM | 984 | ISWSGT | 1258 | AVGSRRLYYSSD | 1532 | Cpa |
| | | | | | IDY | | |
| NBX03177 | SRTFSNYAM | 985 | ISGVTAFT | 1259 | AARQWNPTMR | 1533 | Cpa |
| | | | | | ERDYDY | | |
| NBX03179 | GRSFSRYTM | 986 | ISWSGT | 1260 | AVGTRRLYYSSD | 1534 | Cpa |
| | | | | | IDY | | |
| NBX03180 | GLTVSRYTM | 987 | ISWSGT | 1261 | AAGSRRLHYSS | 1535 | Cpa |
| | | | | | DIDY | | |
| NBX03181 | GLTFSRYTM | 988 | ISWSGT | 1262 | VVGSRRLYYSSD | 1536 | Cpa |
| | | | | | INY | | |
| NBX0570 | GTGRRFGYM | 989 | ISRAGAT | 1263 | FASVFDAGTY | 1537 | NetB |
| NBX0571 | GSIFSINVM | 990 | ATSGGMT | 1264 | NAAQSRTSRLFP | 1538 | NetB |
| | | | | | DEYDY | | |
| NBX0578 | GRDFSTYVM | 991 | SNWNDGGT | 1265 | AARSGGSYTYT | 1539 | CnaA |
| | | | | | GSYHY | | |
| NBX0579 | RTLYNNAM | 992 | ISSSGIST | 1266 | AASRAATVGVT | 1540 | CnaA |
| | | | | | PQEYAY | | |
| NBX0580 | GRTISNYGV | 993 | SWSASST | 1267 | AMMGASYCAG | 1541 | CnaA |
| | | | | | YRCHHAAQTYD | | |
| | | | | | Y | | |
| NBX0581 | GPPSRRYDM | 994 | VSWTGSST | 1268 | AVKNTYGSSSYY | 1542 | CnaA |
| | | | | | YTSSSYDY | | |
| NBX0582 | GEIDSAAGM | 995 | VTWDGGTT | 1269 | AAGNTGPFNLL | 1543 | CnaA |
| | | | | | YSSAQYLY | | |
| NBX0584 | GSMFSIHDM | 996 | VSSDGTT | 1270 | MGNLRRLDSGY | 1544 | CnaA |
| | | | | | ADSYYYK | | |
| NBX0585 | GRTADRYAI | 997 | IERSGGAT | 1271 | AARLALAGEYD | 1545 | CnaA |
| | | | | | Y | | |
| NBX0586 | GGSIRTDRYYW | 998 | IAWTAST | 1272 | VPIVGDSSWSG | 1546 | Cpa |
| | | | | | GS | | |
| NBX0587 | GIIFSINAM | 999 | ITGRGRT | 1273 | NEARNGLGSPA | 1547 | Cpa |
| | | | | | NS | | |
| NBX08116 | GSIFSIYTM | 1000 | ITSGGTTNY | 1274 | NALVNQRDGLA | 1548 | NetB |
| | | | | | TYY | | |
| NBX08117 | GSISSISGM | 1001 | ITSYGST | 1275 | HAVQGSTWW | 1549 | NetB |
| | | | | | GSGS | | |
| NBX08118 | SIGSIHRM | 1002 | ITNGGST | 1276 | YANIREYYSSYEY | 1550 | NetB |
| NBX08119 | GRTFSSYAM | 1003 | INRSGGTT | 1277 | AADRVGDDYYY | 1551 | NetB |
| | | | | | ISSQHYDY | | |
| NBX08120 | GTRGSINPV | 1004 | STGGAT | 1278 | NTESY | 1552 | NetB |
| NBX08121 | GRTFNGKGM | 1005 | INWSGDST | 1279 | AVSTYSTYWFTP | 1553 | NetB |
| | | | | | ARYDY | | |
| NBX08122 | GMTFSDHAM | 1006 | IGRGGTT | 1280 | NVVPLRAGIDTY | 1554 | NetB |
| NBX08123 | GSGRRVGYM | 1007 | ISRAGAT | 1281 | FASIIDAGTY | 1555 | NetB |
| NBX08124 | GIISSITYM | 1008 | IARSGAT | 1282 | YIDQRDVVPT | 1556 | NetB |
| NBX08125 | QSIFRINAM | 1009 | ITSGGVT | 1283 | HARSWSADY | 1557 | NetB |
| NBX08126 | GIMLRDEAL | 1010 | ITRGGAT | 1284 | RIIGTDT | 1558 | NetB |
| NBX08127 | GDTISSYTM | 1011 | ITWSGEVT | 1285 | AAGRAGTNWN | 1559 | NetB |
| | | | | | Y | | |
| NBX08128 | RIISSITDM | 1012 | RGGRI | 1286 | SALSSGKYY | 1560 | NetB |
| NBX08129 | APLNSIHAM | 1013 | STNGGST | 1287 | YAEVRHYSGNV | 1561 | NetB |
| | | | | | DQY | | |
| NBX08138 | GRIFGINGW | 1014 | LYRSGGT | 1288 | NVNWAESDS | 1562 | CnaA |
| NBX08139 | RGTFSNLGL | 1015 | LKRDGNRT | 1289 | AARLPRPNAVV | 1563 | CnaA |
| | | | | | DTTSEYDY | | |
| NBX08140 | GRTFNM | 1016 | ISGSGGIT | 1290 | AVARFGGAWV | 1564 | CnaA |
| | | | | | Y | | |
| NBX08141 | GRTFSDLGM | 1017 | ILSGTTRP | 1291 | AARTVGGLPI | 1565 | CnaA |
| NBX08142 | DTIFSSNGM | 1018 | LYRDGNT | 1292 | NINWPLHDS | 1566 | CnaA |
| NBX08143 | RGTFNNYGL | 1019 | LTRLGGRT | 1293 | AARLPRTNAVV | 1567 | CnaA |
| | | | | | DDISEYTY | | |
| NBX08144 | RGTFNNLGM | 1020 | LKRDGIRT | 1294 | AARLPRTNAVV | 1568 | CnaA |
| | | | | | DSTSEYDY | | |
| NBX08145 | GRIFGINGW | 1021 | YRSGGT | 1295 | NVNWAESDS | 1569 | CnaA |
| NBX08149 | GSPLSINVM | 1022 | ITSGGQT | 1296 | NARDATYYVEY | 1570 | NetB |
| | | | | | NY | | |
| NBX08150 | ARTFSDYAM | 1023 | IRENGGRT | 1297 | AAGRDRYLRFSP | 1571 | NetB |
| | | | | | DY | | |
| NBX08151 | GTIGSINPV | 1024 | STAGAT | 1298 | NTESY | 1572 | NetB |
| NBX08152 | GRTFSGYGM | 1025 | TWNEGST | 1299 | AASTAGYGLGS | 1573 | NetB |
| | | | | | SPNEYEY | | |
| NBX08153 | GRTFTRYGM | 1026 | INGRGDST | 1300 | AVSFTSTFWFSP | 1574 | NetB |
| | | | | | AQYDY | | |
| NBX08154 | GSLFSFETM | 1027 | TTGASP | 1301 | VRERVGSKEY | 1575 | NetB |
| NBX08155 | GLIFSASPM | 1028 | ISASGMIT | 1302 | NAPRANSDWP | 1576 | NetB |
| | | | | | RENS | | |
| NBX08156 | GFTLSSYYI | 1029 | FSNSDGNI | 1303 | AVGYACDYHEL | 1577 | NetB |
| | | | | | RQRFSS | | |
| NBX08157 | RSISSIQAM | 1030 | ITNGGTT | 1304 | NADERPYYGDA | 1578 | NetB |
| | | | | | INS | | |
| NBX11001 | GFAFDTSPM | 1031 | IFSDGST | 1305 | AMSGV | 1579 | CnaA |
| NBX11002 | GFTLDTYAV | 1032 | ISASGSMT | 1306 | AASTRPLCSRGG | 1580 | CnaA |
| | | | | | NYDY | | |
| NBX11003 | GFTLDYSAI | 1033 | ISSSGAYT | 1307 | AARSGVCSRSSS | 1581 | CnaA |
| | | | | | DFGS | | |
| NBX11004 | GGSITTNPYYW | 1034 | IDYSGST | 1308 | ARSLRPVVTAKR | 1582 | CnaA |
| | | | | | DDEY | | |
| NBX11005 | GSIFSINAM | 1035 | INWSGGIT | 1309 | AADLRVGASGPI | 1583 | CnaA |
| | | | | | SFAWAYDY | | |
| NBX11006 | GFSLDNYDI | 1036 | ISRSGGTT | 1310 | APVDITGPGRC | 1584 | CnaA |
| | | | | | PPARYEYGY | | |
| NBX11007 | GFAFDRSAM | 1037 | IFSDGST | 1311 | ATSGI | 1585 | CnaA |
| NBX11008 | GFAFDSSAM | 1038 | IFKDGST | 1312 | ATSGI | 1586 | CnaA |
| NBX11009 | GIGPNAI | 1039 | ISRSGGYT | 1313 | AADAGNFPSRN | 1587 | CnaA |
| | | | | | PVTYGY | | |
| NBX11010 | GFAFDRSPM | 1040 | IFSDGST | 1314 | ATSGI | 1588 | CnaA |
| NBX11011 | GFTLDYYAI | 1041 | ISSSGAYE | 1315 | AAARGGCPPNT | 1589 | CnaA |
| | | | | | YYSGSSYFWEY | | |
| | | | | | DF | | |
| NBX11012 | GFNLNYYDI | 1042 | ISRSGGTE | 1316 | APVDITGPGRC | 1590 | CnaA |
| | | | | | PPARYAYGS | | |
| NBX11013 | GFAFDSSPM | 1043 | IFKDGST | 1317 | ATSGI | 1591 | CnaA |
| NBX11014 | GFAFDSSPM | 1044 | IFKDGTT | 1318 | AMSGV | 1592 | CnaA |
| NBX11015 | GFAFDTSPM | 1045 | IFKDGST | 1319 | ATSGI | 1593 | CnaA |
| NBX11016 | GFNLDNYHI | 1046 | ISSSGGLT | 1320 | GRVNYCARDM | 1594 | CnaA |
| | | | | | SAYDT | | |
| NBX11017 | GFTRDYYTI | 1047 | ISSSDGWT | 1321 | AAAYDTAGWG | 1595 | CnaA |
| | | | | | AGGMDY | | |
| NBX11018 | GSIFSINAM | 1048 | ITWSGGST | 1322 | AADTRRWAGG | 1596 | CnaA |
| | | | | | SSWYGEEYDY | | |
| NBX11019 | GFTLDYYTI | 1049 | ISRSGAMT | 1323 | AATVKSVCFRG | 1597 | CnaA |
| | | | | | ELYDY | | |
| NBX11020 | EFSLANYAI | 1050 | ISSSGNYM | 1324 | ARARSGCSRNM | 1598 | CnaA |
| | | | | | YDSTDY | | |
| NBX11021 | GFTLDYYAI | 1051 | ISSSDGST | 1325 | AAIGSGPLTAQ | 1599 | CnaA |
| | | | | | GMCVMTRTPR | | |
| | | | | | DYDY | | |
| NBX11022 | GFAFDRSPM | 1052 | SLPDGST | 1326 | ATSGV | 1600 | CnaA |
| NBX11023 | GFAFDRSAM | 1053 | IFSDGTT | 1327 | ATSGI | 1601 | CnaA |
| NBX11024 | GFTFDSYVM | 1054 | ISQGGGAT | 1328 | AKGGLADGTGF | 1602 | CnaA |
| | | | | | YGSPGALI | | |
| NBX11025 | GFTFNTYVM | 1055 | ISQGGAAT | 1329 | AQGGLADGSGF | 1603 | CnaA |
| | | | | | YGSPGALI | | |
| NBX11026 | GFTLDYYAI | 1056 | IMSSDGYT | 1330 | AAAYDTAGWG | 1604 | CnaA |
| | | | | | ADGMDY | | |
| NBX11027 | GFAFDTSPM | 1057 | IFEDGTT | 1331 | ATSGI | 1605 | CnaA |
| NBX11028 | GSIFSINGM | 1058 | ISWSGSIT | 1332 | AADIRVGASGPI | 1606 | CnaA |
| | | | | | SFAWAYDY | | |
| NBX11029 | GFILDYYAI | 1059 | ISSSDGST | 1333 | AAVGSGPLSAQ | 1607 | CnaA |
| | | | | | GMCVMTRTSR | | |
| | | | | | DADY | | |
| NBX11032 | GFTLDGYAI | 1060 | ISPSGGMT | 1334 | KVMPLCDRMW | 1608 | CnaA |
| | | | | | SPLGGS | | |
| NBX11033 | GFTLEAYAI | 1061 | ISRSGGLT | 1335 | AAKQTQCSRW | 1609 | CnaA |
| | | | | | NPEYEY | | |
| NBX11034 | GFTFEASAM | 1062 | IFSDGTT | 1336 | ATSGI | 1610 | CnaA |
| NBX11035 | GGSITTNSYYW | 1063 | IDYSGST | 1337 | ARSLRPVVTAKR | 1611 | CnaA |
| | | | | | DDEY | | |
| NBX11036 | GFTFDAYVM | 1064 | ISQGGAAT | 1338 | AKGGLADGSGF | 1612 | CnaA |
| | | | | | YGSPGALI | | |
| NBX11037 | GFAFEASAM | 1065 | IFSDGTT | 1339 | ATSGI | 1613 | CnaA |
| NBX11038 | GGSITTNSYYW | 1066 | IDYSGNT | 1340 | ARSVRPVVTAK | 1614 | CnaA |
| | | | | | GDNEY | | |
| NBX11039 | GFAFDTSPM | 1067 | SFSDGTT | 1341 | ATSGI | 1615 | CnaA |
| NBX11040 | GGSITTNTYYW | 1068 | IDYSGST | 1342 | ARSVRPVVTAK | 1616 | CnaA |
| | | | | | SDDEY | | |
| NBX11041 | GGSITTNSYYW | 1069 | IDYSGNT | 1343 | ARSVRPVVTAK | 1617 | CnaA |
| | | | | | SDNEY | | |
| NBX11042 | GGSITTNSYYW | 1070 | IDYSGNT | 1344 | ARSVRPVVTAK | 1618 | CnaA |
| | | | | | RDNEY | | |
| NBX11043 | GFAFDSSAM | 1071 | ILSDGST | 1345 | ATSGI | 1619 | CnaA |
| NBX11044 | GFAFDSSPM | 1072 | IFGDGST | 1346 | AMSGV | 1620 | CnaA |
| NBX11045 | GFTLDYYDI | 1073 | IEGSGGST | 1347 | AVASSGSIGLCT | 1621 | CnaA |
| | | | | | VGRNAYNY | | |
| NBX11046 | GISRNAI | 1074 | ISRSGGYT | 1348 | AADAGNFPSRN | 1622 | CnaA |
| | | | | | PVTYGY | | |
| NBX11047 | GFTLDYYAI | 1075 | ISSSDGST | 1349 | AAVGSGPLSAQ | 1623 | CnaA |
| | | | | | GMCVMTRTPR | | |
| | | | | | DYDY | | |
| NBX11048 | GFAFDSSAM | 1076 | IFEDGST | 1350 | TTSGI | 1624 | CnaA |
| NBX11049 | GGSITTNHYYW | 1077 | IDYSGST | 1351 | ARSVRPVVTAK | 1625 | CnaA |
| | | | | | RDDEN | | |
| NBX11050 | GFAFDKSAM | 1078 | VFEDGST | 1352 | TTSGI | 1626 | CnaA |
| NBX11051 | GFTLEAYDI | 1079 | ISRSAGHT | 1353 | AAGQTQCTRW | 1627 | CnaA |
| | | | | | SSEYEY | | |
| NBX11052 | GFTFDRSPM | 1080 | IFEDGTT | 1354 | AMSGV | 1628 | CnaA |
| NBX11053 | GETLENYAI | 1081 | ISASGSTT | 1355 | ARTHQLCPRAR | 1629 | CnaA |
| | | | | | SPYDA | | |
| NBX11054 | GFTLDYYTI | 1082 | IMTSDDYT | 1356 | AAAYDTAGWG | 1630 | CnaA |
| | | | | | ADGMDY | | |
| NBX21001 | GSIFSIKDM | 1083 | ITTGGTT | 1357 | GVLLNYRGPSSA | 1631 | NetB |
| | | | | | SYH | | |
| NBX21002 | RSIFSINVM | 1084 | ITRGGSTY | 1358 | NAVPARDR | 1632 | NetB |
| NBX21003 | GFTFSRAAM | 1085 | IDSDGGTT | 1359 | SLFRGYSAYDLR | 1633 | NetB |
| | | | | | S | | |
| NBX21004 | RSIVSDNVM | 1086 | ITRGGSTY | 1360 | NTVPARDR | 1634 | NetB |
| NBX21005 | GRTFSRYGM | 1087 | ISWSGDST | 1361 | AASYNPGTIATI | 1635 | NetB |
| | | | | | RREYEYDY | | |
| NBX21006 | GRTYSSYAM | 1088 | ISWSGGST | 1362 | AADRRTHVSDR | 1636 | NetB |
| | | | | | LGEYDY | | |
| NBX21007 | GRTFSSYAM | 1089 | ISWSDEDT | 1363 | AADRSYTVMVR | 1637 | NetB |
| | | | | | QMRGMDY | | |
| NBX21008 | RSIVSINVM | 1090 | ITRGGSTY | 1364 | NTVPARDR | 1638 | NetB |
| NBX21009 | RSIFSINVW | 1091 | ITRGGSTY | 1365 | NTVPARDH | 1639 | NetB |
| NBX21010 | GRTFSRYGM | 1092 | ISWSGDST | 1366 | AASYNPGNIATI | 1640 | NetB |
| | | | | | RREYEYDY | | |
| NBX21011 | GRTFSRYGM | 1093 | ISWSGGST | 1367 | AASYNPGNIATI | 1641 | NetB |
| | | | | | SREYEYDY | | |
| NBX21012 | GRTFSRYGM | 1094 | ISWSGGST | 1368 | AASYNPGTIATI | 1642 | NetB |
| | | | | | RREYEYDY | | |
| NBX21013 | GRTFSSYAM | 1095 | IRSGNST | 1369 | AADVVTHLATR | 1643 | NetB |
| | | | | | FYEYDY | | |
| NBX21014 | GRTFSSYAM | 1096 | ISWSGGST | 1370 | AADRLTHYSDY | 1644 | NetB |
| | | | | | PADFGS | | |
| NBX21015 | GRTFSSYAM | 1097 | ISWSGSNT | 1371 | AADRLTHYSDY | 1645 | NetB |
| | | | | | PADFDS | | |
| NBX21016 | RSIFSANVM | 1098 | ITRGSSTY | 1372 | NLVPARDR | 1646 | NetB |
| NBX21023 | GSIFSIRDM | 1099 | ISSGGST | 1373 | SVLTNPGWNRP | 1647 | NetB |
| | | | | | LAYF | | |
| NBX21024 | GPTFSSYAMYA | 1100 | VSLTDGSK | 1374 | AGDLDSDVGYE | 1648 | NetB |
| | M | | | | H | | |
| NBX21025 | GRTFSSYAM | 1101 | ISWSGSNT | 1375 | AADRLTHYSDY | 1649 | NetB |
| | | | | | PADFGS | | |
| NBX21026 | GRTFSRYGM | 1102 | ISWSGGST | 1376 | AASYNPGNIATI | 1650 | NetB |
| | | | | | RREYEYDY | | |
| NBX21027 | RSIFSINVM | 1103 | ITRGGSTY | 1377 | NTVPARDR | 1651 | NetB |
| NBX21028 | GRAFSTYTM | 1104 | ISWTGGST | 1378 | AEKARTAVDVR | 1652 | NetB |
| | | | | | VTSGYDY | | |
| NBX21029 | GRIFSINVM | 1105 | ITRGGSTY | 1379 | NTVPARDR | 1653 | NetB |
| NBX21030 | GITFSSYAM | 1106 | ISTSGGST | 1380 | TARWDYPNTYE | 1654 | NetB |
| | | | | | YDY | | |
| NBX21031 | GGIFSFNVM | 1107 | ITRGGSTY | 1381 | NTVPARDR | 1655 | NetB |
| NBX21032 | GHTFSSYIM | 1108 | IRWSSGTT | 1382 | AADAGRDSIYD | 1656 | NetB |
| | | | | | Y | | |
| NBX21033 | GRTFSSYAM | 1109 | IRSGDST | 1383 | AADVVTHIATRF | 1657 | NetB |
| | | | | | YEYDY | | |
| NBX21034 | GRTFSSYAM | 1110 | ISWSGAST | 1384 | AADRLTHYSDY | 1658 | NetB |
| | | | | | PRDFGS | | |
| NBX21035 | RSIFRENVM | 1111 | ITRGSSTY | 1385 | NLVPARDP | 1659 | NetB |
| NBX21038 | GRTFSMYTM | 1112 | ISGSAGST | 1386 | ASEATHGTNRQ | 1660 | NetB |
| | | | | | LDYDY | | |
| NBX21039 | GGTFSRYGM | 1113 | ISWSGDST | 1387 | ASYNPGNIATIR | 1661 | NetB |
| | | | | | REYEYDY | | |
| NBX21040 | GGTFSRYGM | 1114 | ISWSGDST | 1388 | AASYNPGTIATI | 1662 | NetB |
| | | | | | RREYEYDY | | |
| NBX21041 | RGTFSRYGM | 1115 | ISWSGDST | 1389 | AASYNPGTIPTI | 1663 | NetB |
| | | | | | RREYEYDY | | |
| NBX21042 | GRTFSRLGM | 1116 | ISWSGDMT | 1390 | GASYNPGTIATI | 1664 | NetB |
| | | | | | RREYEYDY | | |
| NBX21043 | GGTFSRYGM | 1117 | ISWSGDSK | 1391 | AASYNPGNIATI | 1665 | NetB |
| | | | | | RREYEYDY | | |
| NBX21044 | GGTFSRYGM | 1118 | ISWSGDST | 1392 | AASYNPGNIATI | 1666 | NetB |
| | | | | | RREYEYDY | | |
| NBX21045 | GSSGSIKDM | 1119 | ISTGGTT | 1393 | SVLLNIRGPSSA | 1667 | NetB |
| | | | | | SYH | | |
| NBX21046 | GSSGSIKDM | 1120 | ITTGGTT | 1394 | NVLLNYRGPSSA | 1668 | NetB |
| | | | | | SYH | | |
| NBX21047 | GRSFSSYTM | 1121 | VTWNGEVP | 1395 | AAGNPGRGYDY | 1669 | NetB |
| NBX21048 | GGTFSRYGM | 1122 | ISWSGGST | 1396 | AASYNPGNIATI | 1670 | NetB |
| | | | | | RREYEYDY | | |
| NBX21049 | GRTFSNYAM | 1123 | ISRSGGST | 1397 | AADSLRTHVSD | 1671 | NetB |
| | | | | | RSYEYDS | | |
| NBX21050 | GGTFSRYGM | 1124 | ISWSGDNT | 1398 | ASYNPGNIATIR | 1672 | NetB |
| | | | | | REYEYDY | | |
| NBX21051 | GRTFSSYAM | 1125 | ISRSGAST | 1399 | AADSLRTHVSH | 1673 | NetB |
| | | | | | VSYEYDS | | |
| NBX21052 | GRTFSSYAM | 1126 | IARSGSST | 1400 | AADSLRTHVSD | 1674 | NetB |
| | | | | | RSDEYDY | | |
| NBX21053 | GRTFSRYGM | 1127 | ISWSGDST | 1401 | GASYNPGNIATI | 1675 | NetB |
| | | | | | RREYEYDY | | |
| NBX21054 | GRTFSHYAM | 1128 | IRTSAGIT | 1402 | AIDDDYAYYPPS | 1676 | NetB |
| | | | | | LLDRYDY | | |
| NBX21055 | GRTASSAAM | 1129 | ISWSGGST | 1403 | AADRRTHVSDR | 1677 | NetB |
| | | | | | LGEYDY | | |
| NBX21056 | GRTFSTYAM | 1130 | ISRSGAST | 1404 | AADSLRTHVSH | 1678 | NetB |
| | | | | | VSYEYDS | | |
| NBX21057 | GRTLSRYGM | 1131 | ISWSGDRT | 1405 | ASYNPGNIATIR | 1679 | NetB |
| | | | | | REYEYDY | | |
| NBX21068 | GGTFSRYGL | 1132 | ISWSGDRT | 1406 | GASYNPGNIATI | 1680 | NetB |
| | | | | | RREYEYDY | | |
| NBX21069 | EGTFSRYGM | 1133 | ISWSGDST | 1407 | AASYNPGNIATI | 1681 | NetB |
| | | | | | RREYEYDY | | |
| NBX21070 | GRTFSMYTM | 1134 | ISGSAGST | 1408 | SSTATHGTNRQ | 1682 | NetB |
| | | | | | LDYDY | | |
| NBX21071 | GRTFSTGAM | 1135 | ISWSGGST | 1409 | AASLLTHVSDRA | 1683 | NetB |
| | | | | | LEYDY | | |
| NBX21072 | EGTFSRYGM | 1136 | ISWSGDRT | 1410 | AASYNPGNIATI | 1684 | NetB |
| | | | | | RREYEYDY | | |
| NBX21073 | GSIFSINVM | 1137 | ATSGGQT | 1411 | NGRSASYYPTY | 1685 | NetB |
| | | | | | DY | | |
| NBX21074 | GRTFSHYAM | 1138 | IRGNAGIT | 1412 | AIDLDYAYYPPS | 1686 | NetB |
| | | | | | LVDRYDY | | |
| NBX21075 | GSSGSMKDM | 1139 | ITTGGTT | 1413 | NVLLNYRGPSSA | 1687 | NetB |
| | | | | | SYH | | |
| NBX21076 | GRTFSHYAM | 1140 | ISRSGGST | 1414 | AADSLRTHVSD | 1688 | NetB |
| | | | | | RSYEYDS | | |
| NBX21077 | GRTFSTYAM | 1141 | ISRSGGST | 1415 | AADSLRTHVSD | 1689 | NetB |
| | | | | | RSYEYDY | | |
| NBX21078 | GSIFSSDDM | 1142 | ITSGGMT | 1416 | NAFRSKVVDGII | 1690 | NetB |
| | | | | | LKRRDYDY | | |
| NBX21079 | GRTFSSYTM | 1143 | ISGSAGST | 1417 | ASEATHGTNRQ | 1691 | NetB |
| | | | | | LDYDY | | |
| NBX21080 | GSIFSDDIW | 1144 | ITRGSRPY | 1418 | NAVPARDR | 1692 | NetB |
| NBX21081 | GSIGSINVM | 1145 | VTSGGMT | 1419 | NGRSASYYPTY | 1693 | NetB |
| | | | | | NY | | |
| NBX21082 | GDTLSRYGM | 1146 | ITWSGSST | 1420 | AGPNLGPITIMR | 1694 | NetB |
| | | | | | DYEYDY | | |
| NBX21083 | GSIFSINVM | 1147 | ITSGGKS | 1421 | GRSASYYPTYDY | 1695 | NetB |
| NBX21084 | GSIGSINVM | 1148 | ATSGGMT | 1422 | NGRSASYYPTY | 1696 | NetB |
| | | | | | NY | | |
| NBX21085 | GGTFSRYGM | 1149 | ISWSGDRT | 1423 | AASYNPGNIATI | 1697 | NetB |
| | | | | | RREYEYDY | | |
| NBX21086 | GSTFSINRV | 1150 | IRSNGIT | 1424 | NDPLAPFSSDAS | 1698 | NetB |
| NBX22019 | GRTFSSYAM | 1151 | ISWSGGIT | 1425 | AADARSDSGSY | 1699 | CnaA |
| | | | | | YYPADFGS | | |
| NBX22020 | GGTFSSYPM | 1152 | ISWSGGTT | 1426 | AAQSGPYGSSS | 1700 | CnaA |
| | | | | | SWEADFGS | | |
| NBX22021 | GRTLSSYIM | 1153 | IAWSGSVT | 1427 | AGDLHGIGYEYK | 1701 | CnaA |
| | | | | | Y | | |
| NBX22022 | GFSLDDYAI | 1154 | ISRTSDGST | 1428 | AAGFVCSGYGG | 1702 | CnaA |
| | | | | | GIRGYEYDY | | |
| NBX22023 | NIFSINLM | 1155 | ITSGGNT | 1429 | HCDWCS | 1703 | CnaA |
| NBX22024 | GGTFSSYPM | 1156 | ISWSGGGT | 1430 | AADSPVGPSGL | 1704 | CnaA |
| | | | | | TTHWGY | | |
| NBX22025 | GRTFSSYVM | 1157 | ISWSGGVT | 1431 | AADARSDSGRY | 1705 | CnaA |
| | | | | | YYPADFGS | | |
| NBX22029 | GRTFSNYVM | 1158 | ISWSGGTT | 1432 | AAEHGAGSRW | 1706 | CnaA |
| | | | | | YFPEDFGS | | |
| NBX22030 | NIFSINLM | 1159 | ITSSGNT | 1433 | HCDWCS | 1707 | CnaA |
| NBX22031 | GGTFSSYPM | 1160 | ISWSGGVT | 1434 | AAQSGPYGSSS | 1708 | CnaA |
| | | | | | SWEADFDS | | |
| NBX22032 | GRTFSSYVM | 1161 | ISWSGGVT | 1435 | AAEVGYGSRWY | 1709 | CnaA |
| | | | | | YETDFGS | | |
| NBX22033 | GRTLSSYAM | 1162 | ISWSGGVT | 1436 | AALPDGRSWY | 1710 | CnaA |
| | | | | | QADY | | |
| NBX22034 | GRTFSSYAM | 1163 | ISWSGGVT | 1437 | AAENGYGSSWY | 1711 | CnaA |
| | | | | | FEADFGS | | |
| NBX22035 | GRTFSGSGM | 1164 | ISWTGSIT | 1438 | ARATMGPTSRS | 1712 | CnaA |
| | | | | | DAYDY | | |
| NBX22036 | NIFSINVM | 1165 | ITSGGNT | 1439 | HCDWCS | 1713 | CnaA |
| NBX22037 | NIFSINVM | 1166 | ITSGGDT | 1440 | HCDWCS | 1714 | CnaA |
| NBX22038 | NIFSINLM | 1167 | ITSGGHT | 1441 | HCDWCS | 1715 | CnaA |
| NBX22039 | GGTFSNYAM | 1168 | ISWSGGVA | 1442 | AAEVGYGTSWY | 1716 | CnaA |
| | | | | | YEADFGS | | |
| NBX22040 | GRTFSGSGM | 1169 | ISWTGSIT | 1443 | ARSTVGPTSRSD | 1717 | CnaA |
| | | | | | AYDY | | |
| NBX22041 | GRTLSDYIM | 1170 | IAWSGSTT | 1444 | AGDLHGIGYEYK | 1718 | CnaA |
| | | | | | Y | | |
| NBX22042 | GRTFSGSPM | 1171 | ISWTGSIT | 1445 | ARSTVGPTSRSD | 1719 | CnaA |
| | | | | | AYDY | | |
| NBX22043 | NVLSINLM | 1172 | ITSGGDT | 1446 | HCDWCS | 1720 | CnaA |
| NBX22044 | GRAFSRYTM | 1173 | IDWSGGIDW | 1447 | AAIDYPGTRPAV | 1721 | CnaA |
| | | | SGGRST | | SANEYDY | | |
| NBX22045 | GRTLSNYIM | 1174 | IAWSGSVT | 1448 | AGDLHGIGYEY | 1722 | CnaA |
| | | | | | AY | | |
| NBX22046 | GRTLSSYIM | 1175 | IGWSGSTT | 1449 | AGDLQGIGYEY | 1723 | CnaA |
| | | | | | HN | | |
| NBX22055 | GGTFSNYVM | 1176 | ISWSGGST | 1450 | AAETGGGSRW | 1724 | CnaA |
| | | | | | YRTADFGS | | |
| NBX22056 | GRTFSTYGM | 1177 | ISASGGGT | 1451 | AADRTTWGRQ | 1725 | CnaA |
| | | | | | TPYDY | | |
| NBX22057 | GRTFSTYAM | 1178 | IRWNTGST | 1452 | ALKRYGGGTSTY | 1726 | CnaA |
| | | | | | DQGYDT | | |
| NBX22058 | GRTFSSGAM | 1179 | ISRSGSST | 1453 | AASLGRAYETSA | 1727 | CnaA |
| | | | | | SGAYDY | | |
| NBX22059 | GRTFRSYGV | 1180 | ISSSGGGT | 1454 | AGAVRDWGRD | 1728 | CnaA |
| | | | | | AIYDS | | |
| NBX22060 | GFTFSRYAM | 1181 | ITSAGGST | 1455 | NAEMYSNADV | 1729 | CnaA |
| | | | | | MFNGY | | |
| NBX22061 | GRTISNYTM | 1182 | INPSGGAT | 1456 | AAGSSHGRRW | 1730 | CnaA |
| | | | | | YIDGGRYDY | | |
| NBX22062 | GFTFSNYAM | 1183 | ITSTGGST | 1457 | NAEIYTHYDVM | 1731 | CnaA |
| | | | | | FNGY | | |
| NBX22063 | GRTFSTYGM | 1184 | ISASGGGT | 1458 | AAARTGWGRQ | 1732 | CnaA |
| | | | | | TTHDY | | |
| NBX22064 | GFTFSSYSM | 1185 | SSGGGDIT | 1459 | MRVSGDY | 1733 | CnaA |
| NBX22065 | GRTFSSGAM | 1186 | ISRSGSGT | 1460 | AASLGRAYETSA | 1734 | CnaA |
| | | | | | SGAYDY | | |
| NBX22066 | GRTFRSYGV | 1187 | ISWSGGGT | 1461 | AGAVGDWGRD | 1735 | CnaA |
| | | | | | AIYDY | | |
| NBX22067 | GRTFSSYGM | 1188 | ISTSGGGT | 1462 | AASSRGWGRG | 1736 | CnaA |
| | | | | | VSYDY | | |
| NBX22068 | GGTFSNYVM | 1189 | ISWSGGNA | 1463 | AAETGYGNRW | 1737 | CnaA |
| | | | | | YAPADFGS | | |
| NBX22069 | GHTFSTYGM | 1190 | ISASGGGT | 1464 | AAARTGWGRQ | 1738 | CnaA |
| | | | | | TTHDY | | |
| NBX22070 | GRTFSSYGM | 1191 | ISRSGGGT | 1465 | AASSTGWGRED | 1739 | CnaA |
| | | | | | SYDY | | |
| NBX22083 | GRTFSSYVM | 1192 | ISWSGGVT | 1466 | AASDSENSGSYY | 1740 | CnaA |
| | | | | | RNQDFGY | | |
| NBX22084 | GRTFSNYAM | 1193 | ISWSGGIT | 1467 | AADRGTDSGSY | 1741 | CnaA |
| | | | | | YYTEDFGS | | |
| NBX22085 | GGTFSSYVM | 1194 | ISWSGGVT | 1468 | AADRTAVVPAQ | 1742 | CnaA |
| | | | | | IRSYNY | | |
| NBX22086 | GRTFSNYIM | 1195 | ISWSGGVT | 1469 | AAEVGYGSSWY | 1743 | CnaA |
| | | | | | YEADFGS | | |
| NBX22087 | GRTFSSYAM | 1196 | ISWSGGIT | 1470 | AADLEGAGNFR | 1744 | CnaA |
| | | | | | EFGS | | |
| NBX22088 | GRTFSSYVM | 1197 | ISWSGGVT | 1471 | AADRTAIVPAQI | 1745 | CnaA |
| | | | | | RSYDY | | |
| NBX22089 | GRTGGSFDM | 1198 | ITWSGGST | 1472 | AAGGFGALGVE | 1746 | CnaA |
| | | | | | HRYRY | | |
| NBX22090 | GNIFSISLM | 1199 | ITSGGNT | 1473 | HCDWCS | 1747 | CnaA |
| NBX22093 | GRTFKIYGV | 1200 | ISSSGGGT | 1474 | AVGGWGRDDI | 1748 | CnaA |
| | | | | | YDY | | |
| NBX22094 | ARTFSEYIM | 1201 | ISSSGATT | 1475 | AANRFLSAARY | 1749 | CnaA |
| | | | | | DRQRYDY | | |
| NBX22095 | GHTFSTYGM | 1202 | ISASGGGT | 1476 | AAARASWGRQ | 1750 | CnaA |
| | | | | | TTHDY | | |
| NBX22096 | GLTLNNYAL | 1203 | ISAGGDII | 1477 | RVSGDY | 1751 | CnaA |
| NBX22097 | GRTFSTYGM | 1204 | ISASGGGT | 1478 | AAARTGWGRQ | 1752 | CnaA |
| | | | | | TTYDY | | |
| NBX22098 | GGSLSNYIV | 1205 | ISWSGEVT | 1479 | AGEHNGRSWY | 1753 | CnaA |
| | | | | | DVGNYAY | | |
| NBX22099 | GRTFSSGAM | 1206 | ISRSGSRT | 1480 | AASLGRGYENS | 1754 | CnaA |
| | | | | | DSGAYDY | | |
| NBX22100 | GFNLNYYAI | 1207 | ISSSDGST | 1481 | AARAGGEFYYC | 1755 | CnaA |
| | | | | | SGDSSADY | | |
| NBX22101 | TFSSGAM | 1208 | ISRSSSRT | 1482 | AASLSRGYETSD | 1756 | CnaA |
| | | | | | SGAYDY | | |
| NBX22102 | GRTFRMYGV | 1209 | ISSSGAGT | 1483 | AGADRDWGRD | 1757 | CnaA |
| | | | | | AIYDY | | |
| NBX22103 | GFTWNNYGM | 1210 | INSGGDII | 1484 | NRISGDY | 1758 | CnaA |
| NBX22104 | TFSFGAM | 1211 | ISRSVSRT | 1485 | AASQGRGYETS | 1759 | CnaA |
| | | | | | ATGAYDY | | |
| NBX22105 | GRTFSDYIM | 1212 | ISWSGGVT | 1486 | AADGRSDSGGS | 1760 | CnaA |
| | | | | | YYPADFGS | | |
| NBX22106 | GFTFDDYAI | 1213 | IERSGSST | 1487 | AGRVCSGYGGR | 1761 | CnaA |
| | | | | | IQGYEYDY | | |
| NBX22107 | TFSFGAM | 1214 | ISRSVSRT | 1488 | AASQGRGYETS | 1762 | CnaA |
| | | | | | DTGAYDY | | |
| NBX22108 | GRTFTMYGV | 1215 | ISSSGGGT | 1489 | AVGGWGRDDI | 1763 | CnaA |
| | | | | | YEY | | |
| NBX22109 | GRTFSSGAM | 1216 | ISRSGSTT | 1490 | AASLGRGYENS | 1764 | CnaA |
| | | | | | DSGAYDY | | |
| NBX22110 | TFSSGAM | 1217 | ISRSSSRT | 1491 | AASLSRAYETSD | 1765 | CnaA |
| | | | | | SGAYDY | | |
| NBX22111 | GRTFSTYGM | 1218 | ISASGGGT | 1492 | AAARTSWGRQ | 1766 | CnaA |
| | | | | | TTHDY | | |
| NBX22112 | GRTFSDYVM | 1219 | ISWSGGVT | 1493 | AADARSDSGR | 1767 | CnaA |
| | | | | | WYYPADFGS | | |
| NBX22119 | GRTFSSYAM | 1220 | ISWSGGIT | 1494 | AADTGADSGSY | 1768 | CnaA |
| | | | | | YYPADFGS | | |
| NBX22120 | GGTFSSYIM | 1221 | ISWSGGVT | 1495 | AADARSDSGRY | 1769 | CnaA |
| | | | | | YYPADFGS | | |
| NBX22121 | GRTLSNYAM | 1222 | ISGSGSNT | 1496 | AAREGLALRYAT | 1770 | CnaA |
| | | | | | YDY | | |
| NBX22122 | NIFSIALM | 1223 | ITSGGNT | 1497 | HCDWCS | 1771 | CnaA |
| NBX22123 | GRTFSTYGM | 1224 | ISASGGGT | 1498 | AVARTGWGRE | 1772 | CnaA |
| | | | | | ATHDY | | |
| NBX22124 | GRTFSDYEM | 1225 | ISGSGGTT | 1499 | AARVPRVGRFD | 1773 | CnaA |
| | | | | | ENEYEY | | |
| NBX22125 | GDTFSAYGM | 1226 | ISSGGGGT | 1500 | ARAVRSWGRA | 1774 | CnaA |
| | | | | | AEHDY | | |
| NBX22126 | RTASTSTM | 1227 | WSG | 1501 | AASPRIWYRDT | 1775 | CnaA |
| | | | | | NYKRATWYDY | | |
| NBX22127 | GRTFSDYAM | 1228 | ISWSGGVT | 1502 | AADARSDHASY | 1776 | CnaA |
| | | | | | YYPADFGF | | |
| NBX22128 | GRTFSGYIM | 1229 | ISSSGATT | 1503 | AADQFDSTARY | 1777 | CnaA |
| | | | | | DRRQYEY | | |
| NBX25006 | GSTLSTYAF | 1230 | ISRGGKT | 1504 | DTVGYYPEIR | 1778 | NetB |
| NBX25007 | GSIFGYAM | 1231 | ISRGGTP | 1505 | RCLGFDY | 1779 | NetB |
| NBX25008 | GSIFGIKAM | 1232 | ITEGGTT | 1506 | NSLWSLLEQYP | 1780 | NetB |
| | | | | | RYF | | |
| NBX25009 | GFTPVYYAI | 1233 | ISSVDGKT | 1507 | AAEGPPYDPGQ | 1781 | NetB |
| | | | | | LCPYNDMDY | | |
| NBX25010 | LNSGSIAGM | 1234 | ITRGGSS | 1508 | AYRKDDFGFEV | 1782 | NetB |
| | | | | | LY | | |
| NBX25011 | GSTFSNYAM | 1235 | ISRGGVA | 1509 | DTVGYYSEVR | 1783 | NetB |
| NBX25012 | GSTLSTYAF | 1236 | ISRGGRT | 1510 | DTVGYYPEIR | 1784 | NetB |
| NBX25013 | GITFSAYAM | 1237 | ISRAGIT | 1511 | RTLGFAY | 1785 | NetB |
| NBX25014 | GITFSAYAM | 1238 | ISRAGIT | 1512 | RTLGFPY | 1786 | NetB |
| NBX25015 | GSIAPYAM | 1239 | ISRGSLP | 1513 | TLGFDY | 1787 | NetB |
| NBX25016 | GSTFSSYVM | 1240 | ISSGGST | 1514 | NLRDRFGHGY | 1788 | NetB |
| NBX25017 | GTTLSNYAM | 1241 | ISRGGTP | 1515 | KILGFDV | 1789 | NetB |
| NBX25018 | LNSGSIAGM | 1242 | ITRGGSS | 1516 | FAYRKDDFGFE | 1790 | NetB |
| | | | | | VLY | | |
| NBX0572 | RPTISGYAM | 1796 | ISGSGAST | 1801 | AVYSAPDPTFD | 1806 | CnaA |
| | | | | | HYHMYSANYD | | |
| | | | | | N | | |
| NBX0573 | PTISSYAV | 1797 | SWSASST | 1802 | ASMRASYCAGY | 1807 | CnaA |
| | | | | | RCHORAQTYDY | | |
| NBX0575 | RRTLSSYVM | 1798 | ISKDGRST | 1803 | ASDYNGGGIPSL | 1808 | CnaA |
| | | | | | RLDYNY | | |
| NBX0576 | GSTNTFNKM | 1799 | MSRGDP | 1804 | RGRIGERVY | 1809 | CnaA |
| NBX0577 | GRAIGELSM | 1800 | AIWSGGT | 1805 | NVGANY | 1810 | CnaA |

### 3. Antibodies Recombinantly Expressed

In another aspect, the present invention provides a method for producing V_{H}H in a suitable producing organism. Suitable producing organisms include, without limitation, bacteria, yeast, and algae. In certain embodiments, the producing bacterium is Escherichia coli. In certain embodiments, the producing bacterium is a member of the Bacillus genus. In certain embodiments, the producing bacterium is a probiotic. In certain embodiments, the yeast is Pichia pastoris. In certain embodiments, the yeast is Saccharomyces cerevisiae. In certain embodiments, the alga is a member of the Chlamydomonas or Phaeodactylum genera.

### 3. Antibodies Added to Feed

In yet another aspect, the present invention provides a polypeptide or pluralities thereof comprising a V_{H}H or V_{H}Hs that bind disease-causing agents and are administered to host animals via any suitable route as part of a feed product. In certain embodiments, the animal is selected from the list of host animals described, with that list being representative but not limiting. In certain embodiments, the route of administration to a recipient animal can be, but is not limited to: introduction to the alimentary canal orally or rectally, provided to the exterior surface (for example, as a spray or submersion), provided to the medium in which the animal dwells (including air based media), provided by injection, provided intravenously, provided via the respiratory system, provided via diffusion, provided via absorption by the endothelium or epithelium, or provided via a secondary organism such as a yeast, bacterium, algae, bacteriophages, plants and insects. In certain embodiments, the host is from the superorder Galloanserae. In certain embodiments, the host is a poultry animal. In certain embodiments, the poultry animal is a chicken, turkey, duck, quail, pigeon, squab or goose. In certain embodiments, the poultry animal is a chicken.

### B. Feed Product

In a further aspect, the present invention provides a polypeptide or pluralities thereof comprising a V_{H}H or V_{H}Hs that bind disease-causing agents and are administered to host animals in the form of a product. The form of the product is not limited, so long as it retains binding to the disease-causing agent in the desired form. In certain embodiments, the product is feed, pellet, nutritional supplement, premix, therapeutic, medicine, or feed additive, but is not limited to these forms.

### 1. Feeding Dosage

In a further aspect, the present invention provides a polypeptide or pluralities thereof comprising a V_{H}H or V_{H}Hs that bind disease-causing agents and are administered to host animals as part of a product at any suitable dosage regime. In practice, the suitable dosage is the dosage at which the product offers any degree of protection against a disease-causing agent, and depends on the delivery method, delivery schedule, the environment of the recipient animal, the size of the recipient animal, the age of the recipient animal and the health condition of the recipient animal among other factors. In certain embodiments, V_{H}Hs are administered to recipient animals at a concentration in excess of 1 mg/kg of body weight. In certain embodiments, V_{H}Hs are administered to recipient animals at a concentration in excess of 5 mg/kg of body weight. In certain embodiments, V_{H}Hs are administered to recipient animals at a concentration in excess of 10 mg/kg of body weight. In certain embodiments, V_{H}Hs are administered to recipient animals at a concentration in excess of 50 mg/kg of body weight. In certain embodiments, V_{H}Hs are administered to recipient animals at a concentration in excess of 100 mg/kg of body weight. In certain embodiments, V_{H}Hs are administered to recipient animals at a concentration less than 1 mg/kg of body weight. In certain embodiments, V_{H}Hs are administered to recipient animals at a concentration less than 500 mg/kg of body weight. In certain embodiments, V_{H}Hs are administered to recipient animals at a concentration less than 100 mg/kg of body weight. In certain embodiments, V_{H}Hs are administered to recipient animal at a concentration less than 50 mg/kg of body weight. In certain embodiments, V_{H}Hs are administered to recipient animals at a concentration less than 10 mg/kg of body weight.

### 2. Feeding Frequency

In a further aspect, the present invention provides a polypeptide or pluralities thereof comprising a V_{H}H or V_{H}Hs that bind disease-causing agents and are administered to host animals as part of a product at any suitable dosage frequency. In practice, the suitable dosage frequency is that at which the product offers any protection against a disease-causing agent, and depends on the delivery method, delivery schedule, the environment of the recipient animal, the size of the recipient animal, the age of the recipient animal and the health condition of the recipient animal, among other factors. In certain embodiments, the dosage frequency can be but is not limited to: constantly, at consistent specified frequencies under an hour, hourly, at specified frequencies throughout a 24-hour cycle, daily, at specified frequencies throughout a week, weekly, at specified frequencies throughout a month, monthly, at specified frequencies throughout a year, annually, and at any other specified frequency greater than 1 year.

### 3. Feed Additives

In a further aspect, the present invention provides a polypeptide or pluralities thereof comprising a V_{H}H or V_{H}Hs that bind disease-causing agents and are administered to host animals as part of a product that also comprises other additives or coatings. In practice, the most suitable coating or additive depends on the method of delivery, the recipient animal, the environment of the recipient, the dietary requirements of the recipient animal, the frequency of delivery, the age of the recipient animal, the size of the recipient animal, the health condition of the recipient animal In certain embodiments, these additives and coatings can include but are not limited to the following list and mixtures thereof: a vitamin, an antibiotic, a hormone, an antimicrobial peptide, a steroid, a probiotic, a probiotic, a bacteriophage, chitin, chitosan, B-1,3-glucan, vegetable extracts, peptone, shrimp meal, krill, algae, B-cyclodextran, alginate, gum, tragacanth, pectin, gelatin, an additive spray, a toxin binder, a short chain fatty acid, a medium chain fatty acid, yeast, a yeast extract, sugar, a digestive enzyme, a digestive compound, an essential mineral, an essential salt, or fibre.

### C. Non-Feed Uses

In a further aspect, the present invention provides a polypeptide or pluralities thereof comprising a V_{H}H or V_{H}Hs that bind disease-causing agents, and can be used in a non-feed use, such as but not limited to: a diagnostic kit, an enzyme-linked immunosorbent assay (ELISA), a western blot assay, an immunofluorescence assay, or a fluorescence resonance energy transfer (FRET) assay, in its current form and/or as a polypeptide conjugated to another molecule. In certain embodiments, the conjugated molecule is can be but is not limited to: a fluorophore, a chemiluminescent substrate, an antimicrobial peptide, a nucleic acid, or a lipid.

### D. Antigens

In a further aspect, the present invention provides a polypeptide or pluralities thereof comprising a V_{H}H or V_{H}Hs that bind disease-causing agents, including toxins, produced by a species of Clostridium. In certain embodiments, the species does not belong to the Clostridium genus but is capable of harbouring disease-causing agents shared by Clostridium species. In certain embodiments, the Clostridium species refers to both current and reclassified organisms. In certain embodiments, the Clostridium species is Clostridium perfringens.

In certain embodiments, the V_{H}H or plurality thereof is capable of binding to one or more disease-causing agents, originating from the same or different species. In certain embodiments, the disease-causing agent is a polypeptide with 80% or greater amino acid sequence identity to NetB (SEQ ID NO: 207). In certain embodiments, the disease-causing agent is a polypeptide with 80% or greater amino acid sequence identity to Cpa (SEQ ID NO: 208). In certain embodiments, the disease-causing agent is a polypeptide with 80% or greater amino acid sequence identity to Cpb2 (SEQ ID NO: 209). In certain embodiments, the disease-causing agent is a polypeptide with 80% or greater amino acid sequence identity to CnaA (SEQ ID NO: 210). In certain embodiments, the disease-causing agent is a polypeptide with 80% or greater amino acid sequence identity to the collagen-binding domain of CnaA (SEQ ID NO: 211). In certain embodiments, the disease-causing agent is an exposed peptide, protein, protein complex, nucleic acid, lipid, or combination thereof, that is associated to the surface of the Clostridium bacterium. In certain embodiments, the disease-causing agent is a pilus, fimbria, flagellum, secretion system or porin. In certain embodiments, the disease-causing agent is the Clostridium bacterium.

In certain embodiments, the disease-causing agent or a derivative thereof can be provided in excess and outcompete the activity of the pathogen expressed disease-causing agent. In certain embodiments, a polypeptide with 80% or greater amino acid sequence identity to CnaA (SEQ ID NO: 210) or the collagen-binding domain of CnaA (SEQ ID NO: 211) can be provided in excess to outcompete the activity of CnaA expressed by the Clostridium perfringens bacterium.

1. Antigen Sequences
a. NetB
>ABW71134.1 necrotic enteritis toxin B
precursor [Clostridium perfringens]
(SEQ ID NO: 207)
MKRLKIISITLVLTSVISTSLFSTQTQVFASELNDINKIELKNLS
GEIIKENGKEAIKYTSSDTASHKGWKATLSGTFIEDPHSDKKTAL
LNLEGFIPSDKQIFGSKYYGKMKWPETYRINVKSADVNNNIKIAN
SIPKNTIDKKDVSNSIGYSIGGNISVEGKTAGAGINASYNVQNTI
SYEQPDFRTIQRKDDANLASWDIKFVETKDGYNIDSYHAIYGNQL
FMKSRLYNNGDKNFTDDRDLSTLISGGFSPNMALALTAPKNAKES
VIIVEYQRFDNDYILNWETTQWRGTNKLSSTSEYNEFMFKINWQD
HKIEYYL
b. Cpa
>WP_057230321.1 phospholipase
[Clostridium perfringens]
(SEQ ID NO: 208)
MKRKICKALICAALATSLWAGASTKVYAWDGKIDGTGTHAMIVTQ
GVSILENDLSKNEPESVRKNLEILKENMHELQLGSTYPDYDKNAY
DLYQDHFWDPDTDNNFSKDNSWYLAYSIPDTGESQIRKFSALARY
EWQRGNYKQATFYLGEAMHYFGDIDTPYHPANVTAVDSAGHVKFE
TFAEERKEQYKINTAGCKTNEDFYADILKNKDFNAWSKEYARGFA
KTGKSIYYSHASMSHSWDDWDYAAKVTLANSQKGTAGYIYRFLHD
VSEGNDPSVGKNVKELVAYISTSGEKDAGTDDYMYFGIKTKDGKT
QEWEMDNPGNDFMTGSKDTYTFKLKDENLKIDDIQNMWIRKRKYT
AFPDAYKPENIKIIANGKVVVDKDINEWISGNSTYNIK
c. Cpb2
>AEP94971.1 Beta2-toxin (plasmid)
[Clostridium perfringens]
(SEQ ID NO: 209)
MKKLIVKSTMMLLFSCLLCLGIQLPNTVKANEVNKYQSVMVQYLE
AFKNYDIDTIVDISKDSRTVTKEEYKNMLMEFKYDPNQKLKSYEI
TGSRKIDNGEIFSVKTEFLNGAIYNMEFTVSYIDNKLMVSNMNRI
SIVNEGKCIPTPSFRTQVCTWDDELSQYIGDAVSFTRSSKFQYSS
NTITLNFRQYATSGSRSLKVKYSVVDHWMWGDDIRASQWVYGENP
DYARQIKLYLGSGETFKNYRIKVENYTPASIKVFGEGYCY
d. CnaA
>ALJ54440.1 putative collagen adhesin
[Clostridium perfringens]
(SEQ ID NO: 210)
MKINKKIFSMLFMVIVLFTCISSNFSVSASSIQRGRDISNEVVTS
LVATPNSINDGGNVQVRLEFKENHQRNIQSGDTITVKWTNSGEVF
FEGYEKTIPLYIKDQNVGQAVIEKTGATLTFNDKIDKLDDVGGWA
TFTLQGRNITSGNHEHTGIAYIISGSKRADVNITKPESGTTSVFY
YKTGSMYTNDTNHVNWWLLVNPSKVYSEKNVYIQDEIQGGQTLEP
DSFEIVVTWYDGYVEKFKGKEAIREFHNKYPNSNISVSENKITVN
ISQEDSTQKFINIFYKTKITNPKQKEFVNNTKAWFKEYNKPAVNG
ESFNHSVQNINADAGVNGTVKGELKIIKTLKDKSIPIKDVQFKMR
RVDNTVIKDGKKELLLTTDDKGIANVKGLPVGKYEVKEISAPEWI
AFNPLIAPKLEFTISDQDTEGKLWAVENELKTISIPVEKVWVGQT
SERAEIKLFADGIEVDKVILNADNNWKHTFENKPEYNSETKQKIN
YSVSETTISGYESNITGDAKNGFIVTNTELPDLTIGKEVIGELGD
KTKVFNFELTLKQADGKPINGKFNYIGSVDDRYKKESIKPSDGEI
TFIEGKATITLSHGQEITIKDLPYGVTYKVMEKEANENGYLTTYN
GNNEVTTGELKQDTKVQVVNNKEFVPTTGISTTTEQGTMVGMVIF
SIGILMVMIVVLLQLNKGLKR
e. CnaA Collagen Binding Domain
(SEQ ID NO: 211)
GRDISNEVVTSLVATPNSINDGGNVQVRLEFKENHQRNIQSGDTI
TVKWTNSGEVFFEGYEKTIPLYIKDQNVGQAVIEKTGATLTFNDK
IDKLDDVGGWATFTLQGRNITSGNHEHTGIAYIISGSKRADVNIT
KPESGTTSVFYYKTGSMYTNDTNHVNWWLLVNPSKVYSEKNVYIQ
DEIQGGQTLEPDSFEIVVTWYDGYVEKFKGKEAIREFHNKYPNSN
ISVSENKITVNISQEDSTQKFINIFYKTKITNPKQKEFVNNTKAW
FKEYNKPAVNGESFNHSVQNINADAGVNGTVK

### Examples

The following illustrative examples are representative of the embodiments of the applications, systems and methods described herein and are not meant to be limiting in any way.

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### 1. Production of Antigens

Recombinant antigens can be purified from an E. coli expression system. For example, an antigen can be expressed at 18° C. in E. coli BL21 (DE3) cells grown overnight in autoinducing media (Formedium). Cells are then lysed by sonication in buffer A (250 mM NaCl, 50 mM CaCl₂), 20 mM Imidazole and 10 mM HEPES, pH 7.4) with 12.5 μg/ml DNase I, and 1× Protease inhibitor cocktail (Bioshop). The lysate is cleared by centrifugation at 22000×g for 30 minutes at 4° C., applied to a 5 ml HisTrap HP column (GE Healthcare) pre-equilibrated with buffer A, washed with ten column volumes of buffer A and eluted with a gradient of 0% to 60% (vol/vol) buffer B (250 mM NaCl, 50 mM CaCl₂, 500 mM imidazole and 10 mM HEPES, pH 7.4). The protein is then dialyzed overnight in the presence of TEV against buffer C (250 mM NaCl, 10 mM HEPES, pH 7.4 and 5 mM β-mercaptoethanol) at 4° C. The dialyzed protein is applied to a HisTrap HP column (GE Biosciences) pre-equilibrated with buffer C. 6×His-tagged TEV and 6×His-tag are bound to the column and the antigen is collected in the flowthrough. The sample is dialyzed overnight against buffer D (5 mM NaCl and 10 mM Tris pH 8.8) and then applied to a 5 ml HiTrap Q HP column (GE Healthcare). The protein is eluted with a gradient of 0% to 50% (vol/vol) buffer E (1.0 M NaCl and 10 mM Tris pH 8.8). Lastly, the eluate is loaded onto a Superdex 75 Increase 10/300 GL gel filtration column (GE Healthcare) using buffer F (400 mM NaCl and 20 mM HEPES pH 7.4). The protein sample is then concentrated to 1 mg/mL using Amicon concentrators with appropriate molecular weight cut-off (MWCO; Millipore). The purified protein is stored at −80° C.

### 2. Production of NBXs and Panning

### Llama Immunisation

A single llama is immunized with purified disease-causing agents, such as the antigens listed, which may be accompanied by adjuvants. The llama immunization is performed using 100 μg of each antigen that are pooled and injected for a total of four injections. At the time of injection, the antigens are thawed, and the volume increased to 1 ml with PBS. The 1 ml antigen-PBS mixture is then mixed with 1 ml of Complete Freund's adjuvant (CFA) or Incomplete Freund's adjuvant (IFA) for a total of 2 ml. A total of 2 ml is immunized per injection. Whole llama blood and sera are then collected from the immunized animal on days 0, 28, 49, 70. Sera from days 28, 49 and 70 are then fractionated to separate V_{H}H from conventional antibodies. ELISA can be used to measure reactivity against target antigens in polyclonal and VHH-enriched fractions. Lymphocytes are collected from sera taken at days 28, 49, and 70.

### Panning

RNA isolated from purified llama lymphocytes is used to generate cDNA for cloning into phagemids. The resulting phagemids are used to transform E. coli TG-1 cells to generate a library of expressed V_{H}H genes. The phagemid library size can be ˜2.5×10⁷ total transformants and the estimated number of phagemid containing V_{H}H inserts can be estimated to be ˜100%. High affinity antibodies are then selected by panning against the antigens used for llama immunization. Two rounds of panning are performed and antigen-binding clones arising from round 2 are identified using phage ELISA. Antigen-binding clones are sequenced, grouped according to their CDR regions, and prioritized for soluble expression in E. coli and antibody purification.

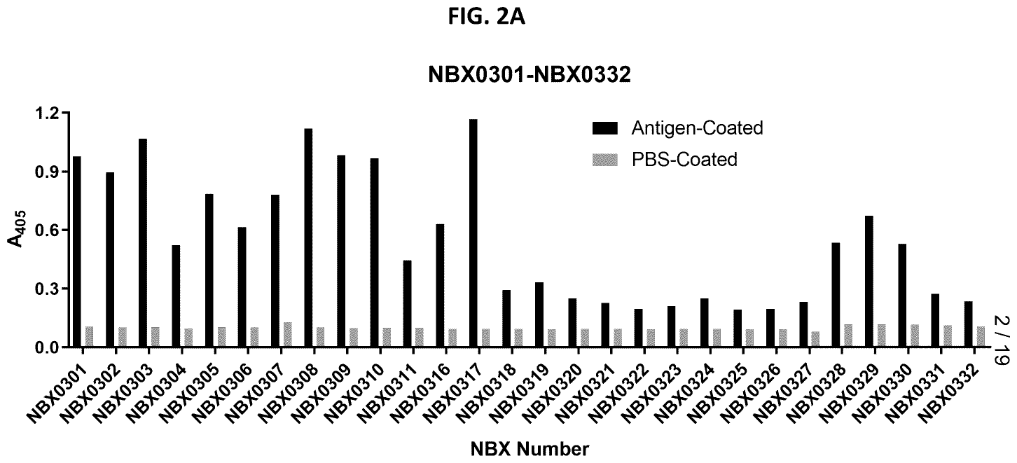

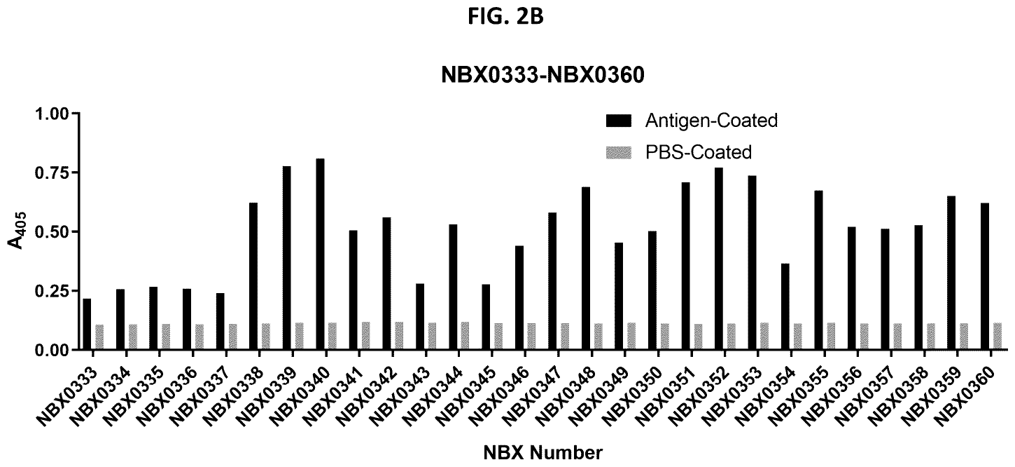

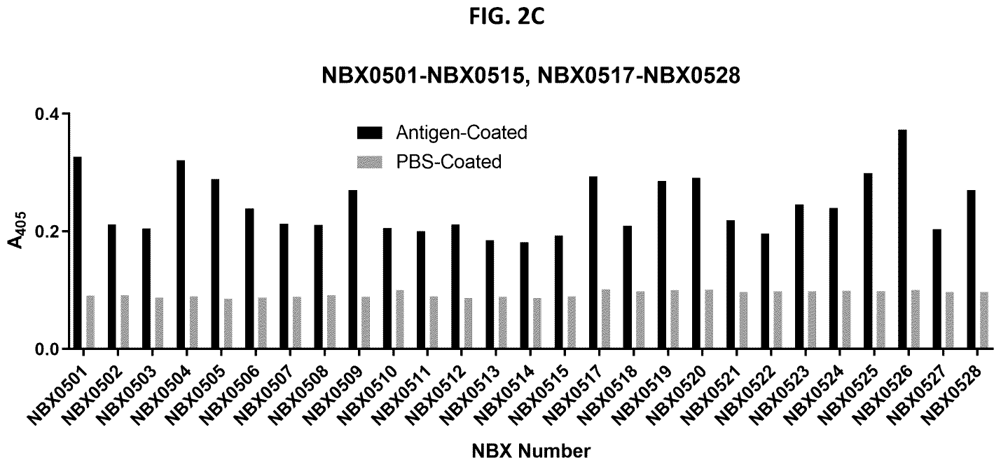

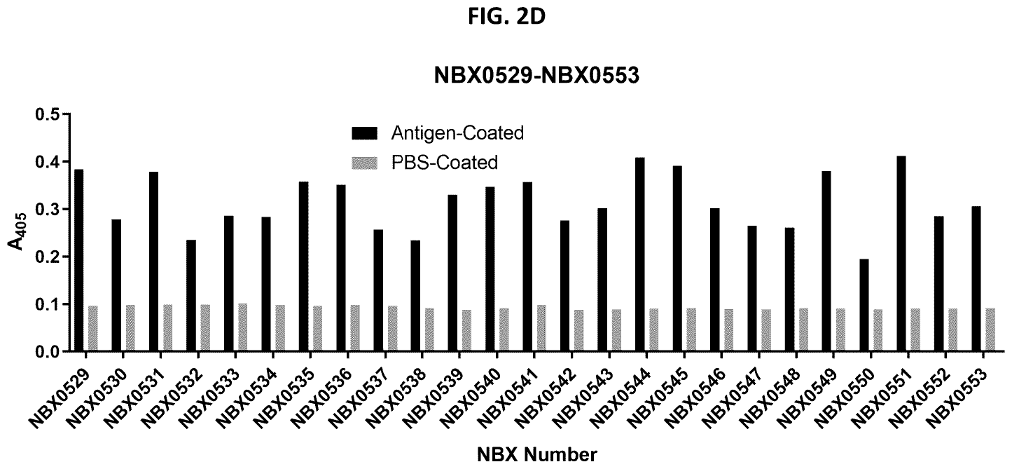

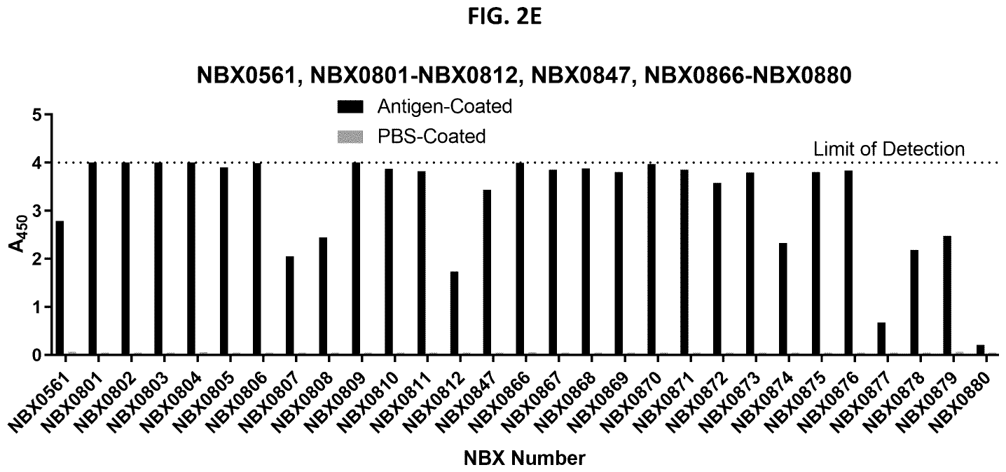

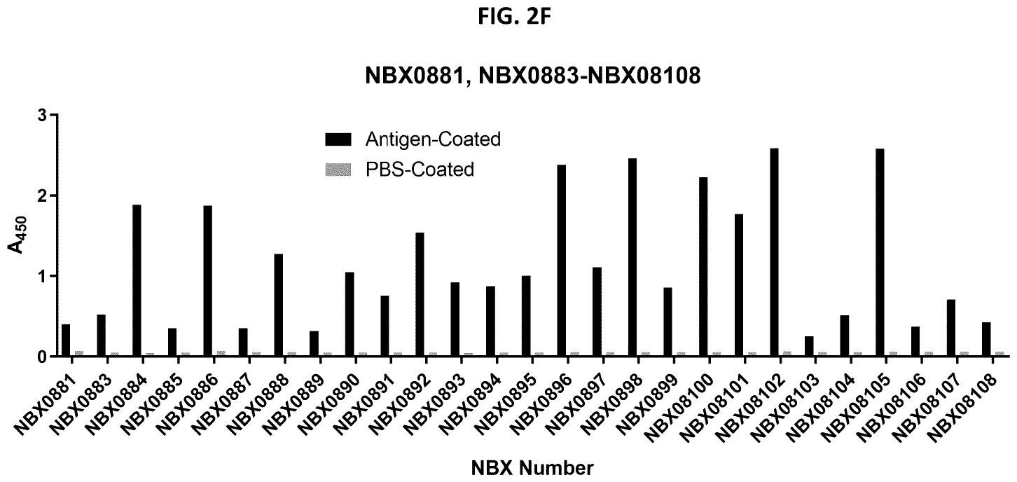

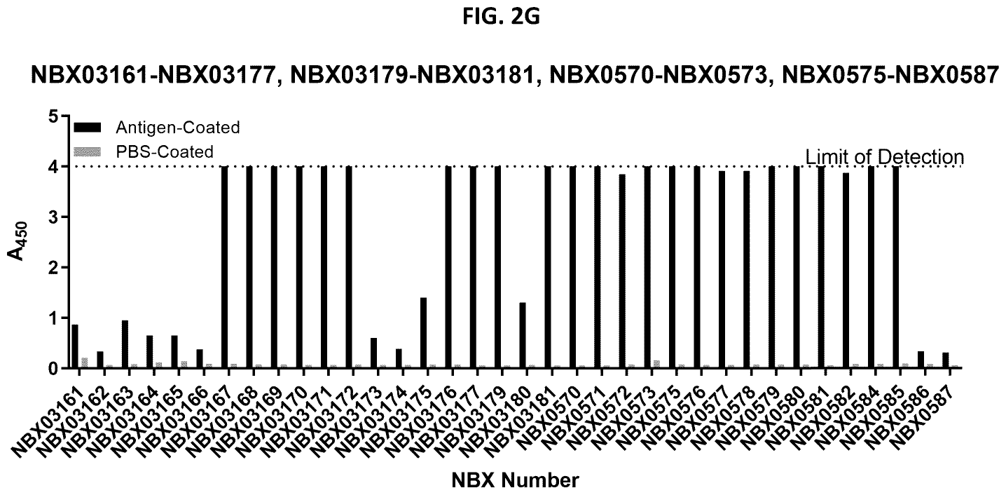

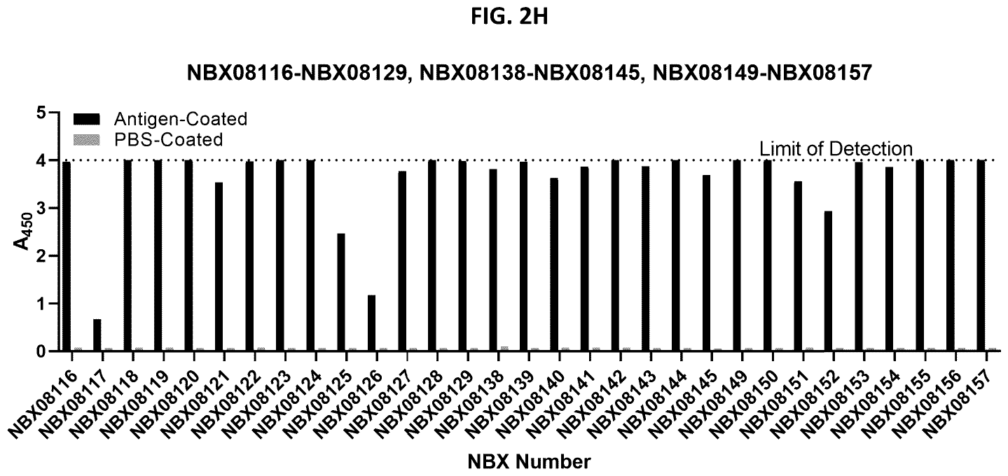

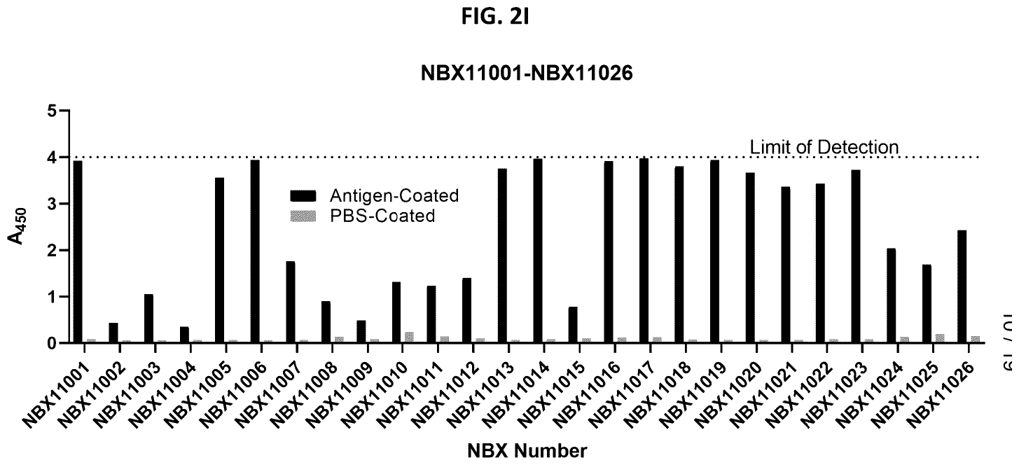

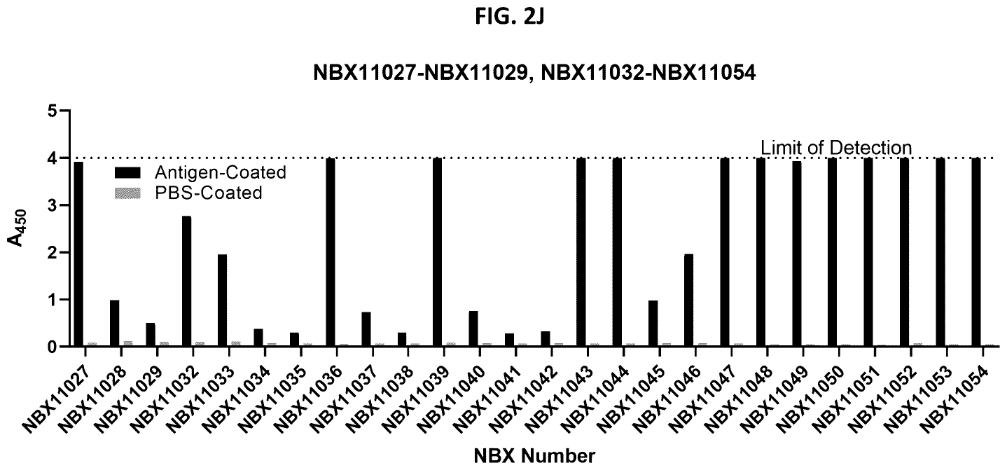

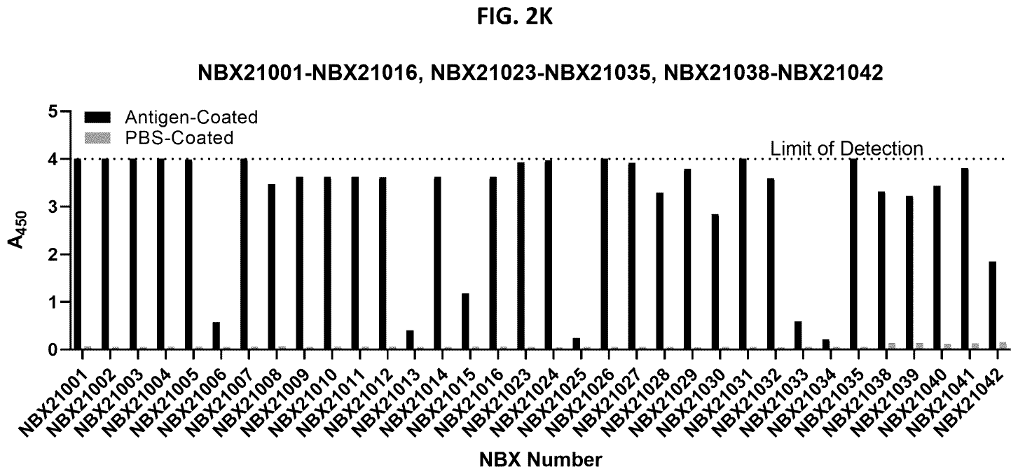

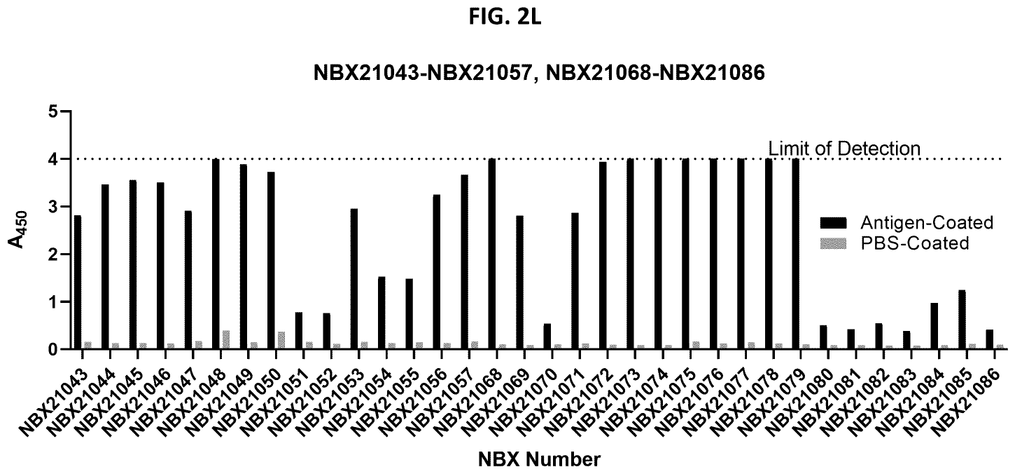

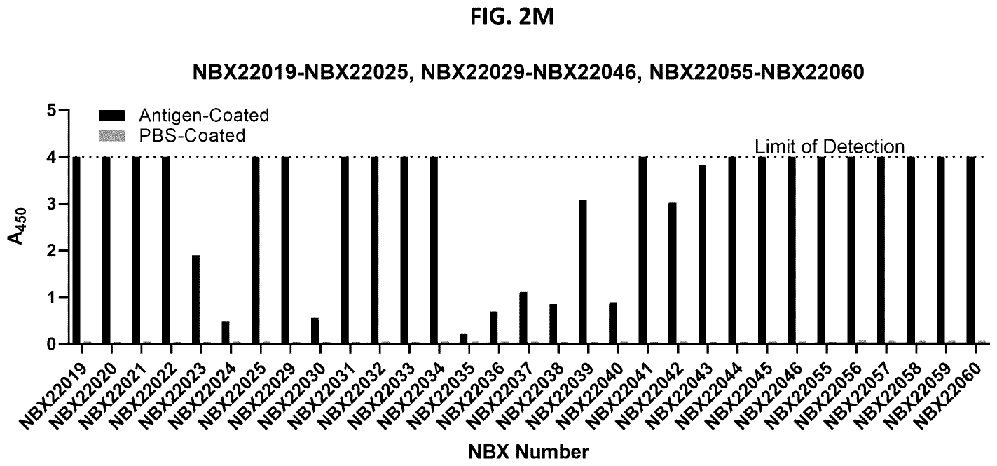

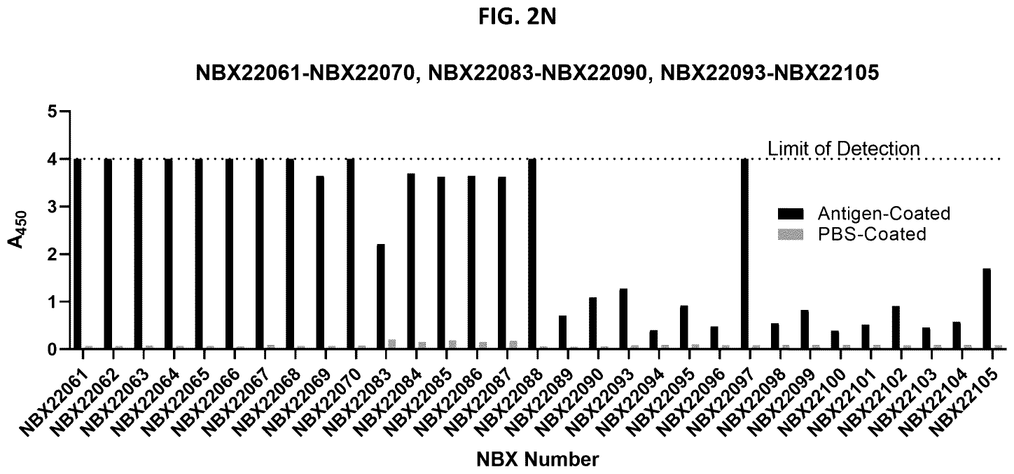

FIG. 2 shows the phage ELISA results for antibodies of this disclosure. Black bars show binding to wells coated with the antigen specified in Tables 1 and 2 dissolved in phosphate-buffered saline (PBS). Grey bars are negative controls that show binding to wells coated with PBS only. In all cases binding to the antigen target is at least twice above binding to the PBS-coated wells. Data for NBX0301 to NBX0332 are shown in panel A. Data for NBX0333-NBX0360 are shown in panel B. Data for NBX0501-NBX0515 and NBX0517-NBX0528 are shown in panel C. Data for NBX0529-NBX0553 are shown in panel D. Data for NBX0561, NBX0801-NBX0812, NBX0847, and NBX0866-NBX0880 are shown in panel E. Data for NBX0881 and NBX0883-NBX08108 are shown in panel F. Data for NBX03161-NBX03177, NBX03179-NBX03181, NBX0570-NBX0573, and NBX0575-NBX0587 are shown in panel G. Data for NBX08116-NBX08129, NBX08138-NBX08145, and NBX08149-NBX08157 are shown in panel H. Data for NBX11001-NBX11026 are shown in panel I. Data for NBX11027-NBX11029 and NBX11032-NBX11054 are shown in panel J. Data for NBX21001-NBX21016, NBX21023-NBX21035, and NBX21038-NBX21042 are shown in panel K. Data for NBX21043-NBX21057 and NBX21068-NBX21086 are shown in panel L. Data for NBX22109-NBX22025, NBX22029-NBX22046, and NBX22055-NBX22060 are shown in panel M. Data for NBX22061-NBX22070, NBX22083-NBX22090, and NBX22093-NBX22105 are shown in panel N. Data for NBX22106-NBX22112, NBX22119-NBX22128, and NBX25006-NBX25018 are shown in panel O.

Purification of V_{H}Hs from E. coli

TEV protease-cleavable, 6×His-thioredoxin-NBX fusion proteins are expressed in the cytoplasm of E. coli grown in autoinducing media (Formedium) for 24 hours at 30° C. Bacteria are collected by centrifugation, resuspended in buffer A (10 mM HEPES, pH 7.5, 250 mM NaCl, 20 mM Imidazole) and lysed using sonication. Insoluble material is removed by centrifugation and the remaining soluble fraction is applied to a HisTrap column (GE Biosciences) pre-equilibrated with buffer A. The protein is eluted from the column using an FPLC with a linear gradient between buffer A and buffer B (10 mM HEPES, pH 7.5, 500 mM NaCl, 500 mM Imidazole). The eluted protein is dialyzed overnight in the presence of TEV protease to buffer C (10 mM HEPES, pH 7.5, 500 mM NaCl). The dialyzed protein is applied to a HisTrap column (GE Biosciences) pre-equilibrated with buffer C. 6×His-tagged TEV and 6×His-tagged thioredoxin are bound to the column and highly purified NBX is collected in the flowthrough. NBX proteins are dialyzed overnight to PBS and concentrated to ˜ 10 mg/ml.

Purification of V_{H}Hs from Pichia Pastoris

Pichia pastoris strain GS115 with constructs for the expression and secretion of 6×His-tagged V_{H}H are grown for 5 days at 30° C. with daily induction of 0.5% (vol/vol) methanol. Yeast cells are removed by centrifugation and the NBX-containing supernatant is spiked with 10 mM imidazole. The supernatant is applied to a HisTrap column (GE Biosciences) pre-equilibrated with buffer A (10 mM HEPES, pH 7.5, 500 mM NaCl). The protein is eluted from the column using an FPLC with a linear gradient between buffer A and buffer B (10 mM HEPES, pH 7.5, 500 mM NaCl, 500 mM Imidazole). NBX proteins are dialyzed overnight to PBS and concentrated to ˜10 mg/ml.

### 3. NBX Neutralization of NetB Cytotoxicity

Hepatocellular carcinoma-derived epithelial cells (LMH cells) from Gallus gallus strain Leghorn are adhered to the surface of a tissue-culture treated and gelatin-coated 96-well microtitre plate at 64,000 cells/well overnight at 37° C. and 5% CO₂. Recombinantly expressed NetB is preincubated with NBX at a range of concentrations or the buffer in which the NBXs are dissolved (20 mM HEPES pH 7.4, 150 mM NaCl) for 15 minutes at 37° C. and 5% CO₂. After 15 minutes the toxin/NBX mixtures are added to triplicate wells of LMH cells. The final concentration of NetB is 5 nM. The final concentrations of NBXs are 1, 3, 9, 27, 81, 243, 729, and 2187 nM. LMH cells with toxin/NBX mixtures are incubated for 5 hours at 37° C. and 5% CO₂. Cytotoxicity induced by NetB is measured using the Pierce LDH Cytotoxicity Assay Kit (Thermo Scientific) following the manufacturer's instructions. NetB percent cytotoxicity in the presence of NBX is determined relative to NetB cytotoxicity in the absence of NBX. A non-linear fit of the inhibitor concentration versus response is determined using GraphPad Prism 8 which generates the 50% inhibitory concentration (IC₅₀) which approximates the NBX concentration required to block 50% of the cytotoxicity of 5 nM NetB.

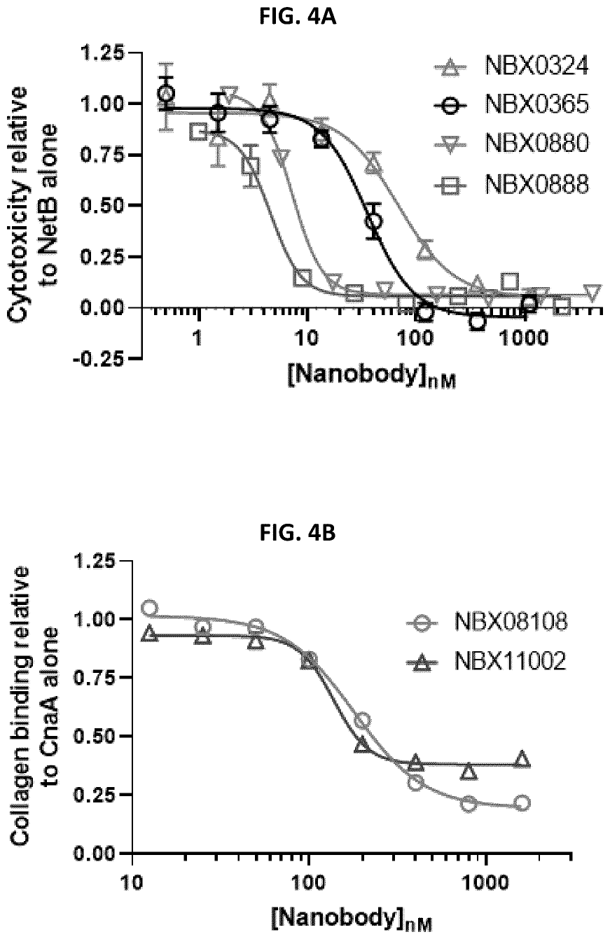

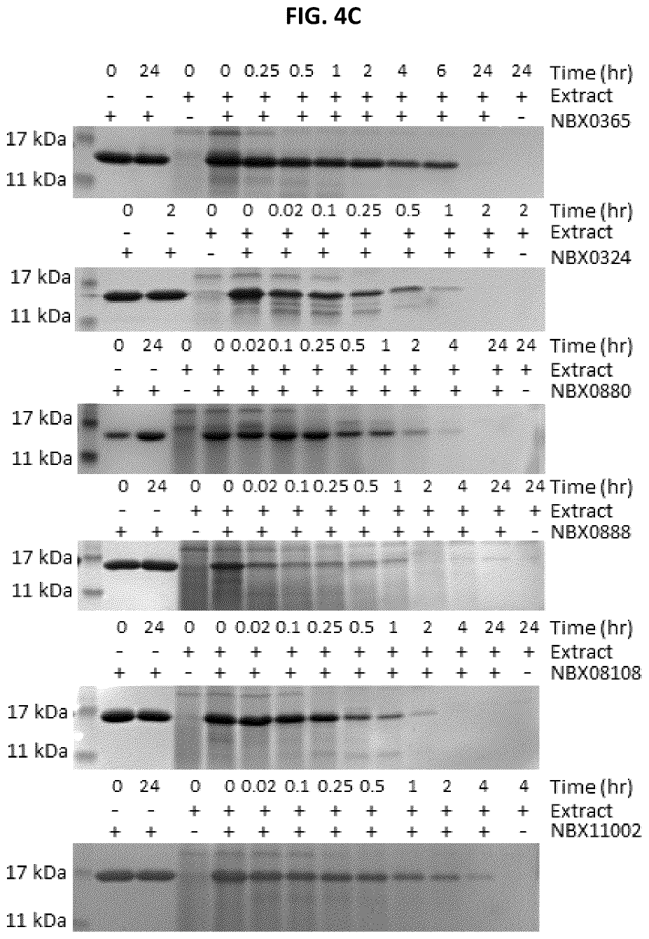

FIG. 4A shows the complete curves for four NBXs (NBX0324, NBX0365, NBX0880, and NBX0888). All four of which have IC₅₀ values below 100 nM and can inhibit NetB cytotoxicity by at least 90%.

Table 3 indicates, for all NBXs tested, whether the NBX can neutralize the activity of NetB against LMH cells with an IC₅₀-value less than 1 uM and/or less than 50 nM.

TABLE 3
Summary table for NBXs that neutralize NetB

| NBX Number | IC₅₀ < 1 μM | IC₅₀ < 50 nM |
|---|---|---|
| NBX0301 | Yes | No |
| NBX0303 | Yes | Yes |
| NBX0304 | No | No |
| NBX0305 | Yes | Yes |
| NBX0307 | Yes | Yes |
| NBX0308 | Yes | No |
| NBX0309 | Yes | Yes |
| NBX0310 | Yes | Yes |
| NBX0311 | Yes | No |
| NBX0318 | Yes | Yes |
| NBX0319 | Yes | Yes |
| NBX0322 | Yes | No |
| NBX0323 | Yes | No |
| NBX0324 | Yes | Yes |
| NBX0362 | Yes | No |
| NBX0364 | Yes | Yes |
| NBX0365 | Yes | Yes |
| NBX0366 | Yes | Yes |
| NBX0370 | Yes | No |
| NBX0371 | Yes | Yes |
| NBX0372 | Yes | No |
| NBX0373 | Yes | No |
| NBX0375 | Yes | Yes |
| NBX0376 | Yes | No |
| NBX0378 | Yes | No |
| NBX0379 | Yes | No |
| NBX0501 | Yes | Yes |
| NBX0502 | No | No |
| NBX0503 | Yes | Yes |
| NBX0504 | No | No |
| NBX0505 | Yes | Yes |
| NBX0506 | Yes | Yes |
| NBX0507 | Yes | Yes |
| NBX0508 | Yes | No |
| NBX0509 | No | No |
| NBX0510 | Yes | Yes |
| NBX0511 | Yes | Yes |
| NBX0512 | Yes | No |
| NBX0513 | Yes | No |
| NBX0538 | Yes | Yes |
| NBX0539 | Yes | Yes |
| NBX0540 | Yes | Yes |
| NBX0541 | Yes | No |
| NBX0542 | Yes | Yes |
| NBX0543 | Yes | No |
| NBX0544 | Yes | Yes |
| NBX0545 | Yes | Yes |
| NBX0546 | Yes | Yes |
| NBX0547 | Yes | No |
| NBX0548 | Yes | Yes |
| NBX0549 | Yes | Yes |
| NBX0550 | Yes | Yes |
| NBX0551 | Yes | Yes |
| NBX0552 | Yes | Yes |
| NBX0553 | Yes | Yes |
| NBX0877 | Yes | Yes |
| NBX0878 | Yes | Yes |
| NBX0879 | Yes | No |
| NBX0880 | Yes | Yes |
| NBX0881 | Yes | Yes |
| NBX0883 | Yes | Yes |
| NBX0884 | Yes | No |
| NBX0885 | Yes | Yes |
| NBX0886 | Yes | Yes |
| NBX0887 | Yes | Yes |
| NBX0888 | Yes | Yes |
| NBX0892 | Yes | Yes |
| NBX0893 | Yes | Yes |
| NBX0894 | Yes | Yes |
| NBX0895 | Yes | Yes |
| NBX08116 | Yes | Yes |
| NBX08117 | No | No |
| NBX08121 | No | No |
| NBX08122 | Yes | No |
| NBX08123 | Yes | Yes |
| NBX08127 | Yes | No |
| NBX08129 | Yes | Yes |
| NBX08149 | Yes | Yes |
| NBX08152 | Yes | Yes |
| NBX08155 | Yes | Yes |
| NBX21001 | Yes | Yes |
| NBX21009 | Yes | Yes |
| NBX21024 | Yes | Yes |
| NBX21029 | Yes | No |
| NBX21043 | Yes | Yes |
| NBX21044 | Yes | Yes |
| NBX21045 | Yes | Yes |
| NBX21046 | No | No |
| NBX21047 | No | No |
| NBX21049 | Yes | No |
| NBX21052 | Yes | Yes |
| NBX21055 | Yes | No |
| NBX21056 | Yes | Yes |
| NBX21057 | Yes | Yes |
| NBX21068 | Yes | Yes |
| NBX21069 | Yes | No |
| NBX21070 | Yes | No |
| NBX21071 | Yes | No |
| NBX21072 | Yes | Yes |
| NBX21073 | Yes | Yes |
| NBX21074 | No | No |
| NBX21075 | Yes | No |
| NBX21076 | No | No |
| NBX21077 | Yes | No |
| NBX21078 | Yes | No |
| NBX21080 | Yes | Yes |
| NBX21081 | Yes | Yes |
| NBX21082 | Yes | Yes |
| NBX21083 | Yes | Yes |
| NBX21084 | Yes | Yes |
| NBX21085 | Yes | Yes |
| NBX21086 | Yes | Yes |
| NBX25006 | No | No |
| NBX25007 | Yes | No |
| NBX25008 | Yes | No |
| NBX25009 | Yes | Yes |
| NBX25010 | Yes | Yes |
| NBX25011 | No | No |
| NBX25012 | No | No |
| NBX25013 | Yes | Yes |
| NBX25014 | Yes | Yes |
| NBX25015 | Yes | No |
| NBX25016 | No | No |
| NBX25017 | Yes | Yes |
| NBX25018 | Yes | No |

### 4. NBX Reduction of CnaA Collagen Binding

In a 96-well microtiter plate, 2 μg of collagen is incubated in 100 μl of PBS per well overnight at 4° C. The plate is washed with 200 μl of PBS and then blocked with 200 μl of 5% skim milk in PBS for 2 hours at 37° C. During the blocking step, 200 nM or 2 uM of individual NBXs are mixed with or without 100 nM of 6×-Histidine and Maltose-binding-protein (MBP) tagged CnaA in PBS for 30 minutes at 37° C. The plate is washed with 200 μl of PBS three times, and 100 μl of NBXs or NBX/MBP-CnaA mixture is added to each well for a 2-hour incubation at 37° C. After washing with 200 μl of PBS three times, 100 μl of 0.125 μg/ml of anti-His conjugated with HRP is added to each well and incubated for 1 hour at room temperature. The plate is then washed with 200 μl of PBS three times, and 100 μl of TMB substrate is added to each well and allowed to develop for 30 minutes. To stop the reaction, 50 μl of 1 M HCl is added to each well. Absorbance of the plate at 450 nm is read to quantify binding. To quantify the reduction of CnaA binding to collagen in the presence of NBX, a percent reduction is calculated relative to the binding of CnaA in the absence of NBX (100% binding).

Table 4 indicates, for all NBXs tested, whether the NBX can reduce binding of CnaA to collagen by more than 50% when the NBX is supplied at 2 μM and/or at 200 nM.

TABLE 4
Summary table for NBXs that neutralize CnaA

| | Collagen-binding | Collagen-binding |
|---|---|---|
| NBX | reduced by >50% | reduced by >50% |
| Number | at 2 μM* | at 200 nM |
| NBX0316 | Yes | Yes |
| NBX0317 | Yes | Yes |
| NBX0325 | Yes | Yes |
| NBX0326 | Yes | Yes |
| NBX0327 | No | No |
| NBX0514 | No | No |
| NBX0515 | No | No |
| NBX0518 | No | No |
| NBX0520 | Yes | No |
| NBX0521 | No | No |
| NBX0522 | Yes | Yes |
| NBX0523 | No | No |
| NBX0524 | No | No |
| NBX0526 | No | No |
| NBX0527 | No | No |
| NBX0528 | Yes | Yes |
| NBX0529 | No | No |
| NBX0530 | Yes | Yes |
| NBX0531 | Yes | Yes |
| NBX0532 | No | No |
| NBX0533 | No | No |
| NBX0534 | Yes | Yes |
| NBX0535 | Yes | Yes |
| NBX0537 | No | No |
| NBX0801 | Yes | No |
| NBX0802 | Yes | No |
| NBX0803 | Yes | Yes |
| NBX0804 | Yes | Yes |
| NBX0805 | No | No |
| NBX0806 | No | No |
| NBX0807 | Yes | Yes |
| NBX0808 | Yes | No |
| NBX0809 | Yes | Yes |
| NBX0811 | Yes | Yes |
| NBX0812 | Yes | Yes |
| NBX0847 | Yes | No |
| NBX0866 | Yes | Yes |
| NBX0867 | Yes | No |
| NBX0868 | Yes | No |
| NBX0869 | Yes | Yes |
| NBX0870 | No | No |
| NBX0871 | No | No |
| NBX0872 | Yes | No |
| NBX0873 | Yes | Yes |
| NBX0874 | Yes | Yes |
| NBX0875 | Yes | Yes |
| NBX0876 | Yes | Yes |
| NBX0896 | Yes | No |
| NBX0897 | Yes | No |
| NBX0898 | Yes | No |
| NBX0899 | Yes | Yes |
| NBX08100 | Yes | Yes |
| NBX08101 | Yes | Yes |
| NBX08102 | Yes | Yes |
| NBX08103 | Yes | No |
| NBX08104 | Yes | Yes |
| NBX08105 | Yes | Yes |
| NBX08106 | Yes | Yes |
| NBX08107 | Yes | Yes |
| NBX08108 | Yes | Yes |
| NBX0517 | N.D. | No |
| NBX0519 | N.D. | No |
| NBX0561 | Yes | No |
| NBX0572 | N.D. | No |
| NBX0573 | N.D. | No |
| NBX0575 | N.D. | Yes |
| NBX0576 | N.D. | No |
| NBX0577 | N.D. | No |
| NBX0578 | N.D. | No |
| NBX0579 | N.D. | No |
| NBX0580 | N.D. | No |
| NBX0581 | N.D. | No |
| NBX0582 | N.D. | No |
| NBX0584 | N.D. | No |
| NBX0585 | N.D. | No |
| NBX08138 | N.D. | Yes |
| NBX08139 | N.D. | No |
| NBX08140 | N.D. | No |
| NBX08141 | N.D. | Yes |
| NBX08142 | N.D. | Yes |
| NBX08143 | N.D. | Yes |
| NBX08144 | N.D. | Yes |
| NBX08145 | N.D. | Yes |
| NBX11001 | N.D. | No |
| NBX11002 | N.D. | Yes |
| NBX11004 | N.D. | Yes |
| NBX11005 | N.D. | No |
| NBX11007 | N.D. | No |
| NBX11008 | N.D. | Yes |
| NBX11009 | N.D. | Yes |
| NBX11010 | N.D. | Yes |
| NBX11011 | N.D. | Yes |
| NBX11012 | N.D. | Yes |
| NBX11013 | N.D. | Yes |
| NBX11014 | N.D. | Yes |
| NBX11015 | N.D. | Yes |
| NBX11016 | N.D. | Yes |
| NBX11017 | N.D. | Yes |
| NBX11018 | N.D. | Yes |
| NBX11019 | N.D. | Yes |
| NBX11020 | N.D. | Yes |
| NBX11021 | N.D. | No |
| NBX11022 | N.D. | Yes |
| NBX11023 | N.D. | No |
| NBX11024 | N.D. | No |
| NBX11025 | N.D. | No |
| NBX11026 | N.D. | No |
| NBX11027 | N.D. | Yes |
| NBX11028 | N.D. | No |
| NBX11029 | N.D. | No |
| NBX11032 | N.D. | Yes |
| NBX11034 | N.D. | No |
| NBX11035 | N.D. | Yes |
| NBX11036 | N.D. | Yes |
| NBX11037 | N.D. | No |
| NBX11038 | N.D. | Yes |
| NBX11039 | N.D. | No |
| NBX11040 | N.D. | Yes |
| NBX11041 | N.D. | Yes |
| NBX11042 | N.D. | Yes |
| NBX11043 | N.D. | Yes |
| NBX11044 | N.D. | Yes |
| NBX11045 | N.D. | No |
| NBX11046 | N.D. | Yes |
| NBX11047 | N.D. | No |
| NBX11048 | N.D. | Yes |
| NBX11049 | N.D. | Yes |
| NBX11050 | N.D. | No |
| NBX11051 | N.D. | No |
| NBX11052 | N.D. | No |
| NBX11053 | N.D. | No |
| NBX11054 | N.D. | No |
| NBX22019 | N.D. | No |
| NBX22020 | N.D. | Yes |
| NBX22021 | N.D. | No |
| NBX22022 | N.D. | No |
| NBX22023 | N.D. | No |
| NBX22024 | N.D. | No |
| NBX22025 | N.D. | No |
| NBX22029 | N.D. | No |
| NBX22030 | N.D. | No |
| NBX22031 | N.D. | Yes |
| NBX22032 | N.D. | No |
| NBX22033 | N.D. | No |
| NBX22034 | N.D. | No |
| NBX22035 | N.D. | No |
| NBX22039 | N.D. | No |
| NBX22040 | N.D. | No |
| NBX22041 | N.D. | No |
| NBX22042 | N.D. | Yes |
| NBX22043 | N.D. | No |
| NBX22044 | N.D. | No |
| NBX22045 | N.D. | No |
| NBX22046 | N.D. | No |
| NBX22055 | N.D. | No |
| NBX22056 | N.D. | Yes |
| NBX22057 | N.D. | No |
| NBX22058 | N.D. | No |
| NBX22059 | N.D. | No |
| NBX22060 | N.D. | No |
| NBX22061 | N.D. | Yes |
| NBX22062 | N.D. | No |
| NBX22063 | N.D. | Yes |
| NBX22064 | N.D. | No |
| NBX22065 | N.D. | Yes |
| NBX22066 | N.D. | No |
| NBX22067 | N.D. | Yes |
| NBX22068 | N.D. | Yes |
| NBX22069 | N.D. | Yes |
| NBX22070 | N.D. | Yes |
| NBX22083 | N.D. | No |
| NBX22084 | N.D. | No |
| NBX22085 | N.D. | No |
| NBX22086 | N.D. | No |
| NBX22087 | N.D. | No |
| NBX22088 | N.D. | No |
| NBX22089 | N.D. | No |
| NBX22090 | N.D. | No |
| NBX22093 | N.D. | Yes |
| NBX22094 | N.D. | Yes |
| NBX22095 | N.D. | Yes |
| NBX22096 | N.D. | No |
| NBX22097 | N.D. | Yes |
| NBX22098 | N.D. | Yes |
| NBX22099 | N.D. | No |
| NBX22100 | N.D. | Yes |
| NBX22101 | N.D. | Yes |
| NBX22102 | N.D. | Yes |
| NBX22103 | N.D. | Yes |
| NBX22104 | N.D. | Yes |
| NBX22105 | N.D. | Yes |
| NBX22106 | N.D. | No |
| NBX22107 | N.D. | Yes |
| NBX22108 | N.D. | No |
| NBX22109 | N.D. | Yes |
| NBX22110 | N.D. | Yes |
| NBX22111 | N.D. | Yes |
| NBX22112 | N.D. | Yes |
| NBX22119 | N.D. | No |
| NBX22120 | N.D. | No |
| NBX22121 | N.D. | Yes |
| NBX22122 | N.D. | Yes |
| NBX22123 | N.D. | Yes |
| NBX22124 | N.D. | No |
| NBX22125 | N.D. | Yes |
| NBX22126 | N.D. | Yes |
| NBX22127 | N.D. | No |
| NBX22128 | N.D. | Yes |

*N.D.: CnaA-Collagen binding reduction not determined at NBX concentration of 2 μM. Binding reduction only determined at NBX concentration of 200 nM.

For a selection of NBXs the CnaA-collagen binding assay was modified to test a larger range of NBX concentrations and calculate IC₅₀ values. The concentrations of NBXs tested were 12.5, 25, 50, 100, 200, 400, 800, and 1600 nM.

FIG. 4B shows the inhibition of CnaA binding to collagen for two NBXs (NBX08108 and NBX11002) which reduced CnaA binding to collagen by at least 50% with IC₅₀ values below 200 nM.

### 5. NBX Neutralization of Cpa Lecithinase Activity

Cpa is mixed with NBX or PBS to achieve a final concentration of 100 nM (Cpa) and 1 uM (NBX) in a total store-bought, free-range eggs by separation from the white. The yolk is punctured carefully then 5 ml is removed and mixed thoroughly with 45 ml PBS to create a 10% solution. The solution is centrifuged at 500 g to remove large aggregates and then passed through a 0.45 um GD/X syringe filter. 60 μl of the filtered yolk solution is added to the Cpa or Cpa/NBX wells to achieve a final concentration of 5% v/v egg yolk. The plate is incubated for 1 hr at 37° C. after which the optical density of the plate is measured at 620 nm. NBX neutralization of Cpa lecithinase activity is determined relative to Cpa lecithinase activity in the absence of NBX (100%).

Table 5 indicates, for all NBXs tested, whether the NBX can reduce Cpa lecithinase activity by more than 40% when the NBX is supplied at 1 uM.

TABLE 5
Summary table for NBXs that neutralize Cpa

| | Cpa lecithinase activity |
|---|---|
| NBX Number | reduced by >40% at 1 μM |
| NBX0329 | Yes |
| NBX0330 | No |
| NBX0338 | Yes |
| NBX0339 | Yes |
| NBX0340 | No |
| NBX0341 | No |
| NBX03161 | No |
| NBX03162 | No |
| NBX03163 | Yes |
| NBX03164 | Yes |
| NBX03165 | Yes |
| NBX03167 | Yes |
| NBX03168 | Yes |
| NBX03169 | Yes |
| NBX03170 | Yes |
| NBX03171 | Yes |
| NBX03172 | Yes |
| NBX03173 | No |
| NBX03174 | No |
| NBX03175 | No |
| NBX03176 | Yes |
| NBX03177 | No |
| NBX03179 | Yes |
| NBX03180 | Yes |
| NBX03181 | Yes |
| NBX0587 | No |

### 6. Untagged CnaA Provided in Excess Outcompetes Tagged CnaA for Collagen Binding

In a 96-well microtiter plate, 2 μg of collagen is incubated in 100 μl of PBS per well overnight at 4° C. The plate is washed with 200 μl of PBS and then blocked with 200 μl of 5% skim milk in PBS for 2 hours at 37° C. During the blocking step, 100 nM of 6×-Histidine and Maltose-binding-protein (MBP) tagged CnaA is mixed with between 0 and 2000 nM untagged CnaA in PBS for 30 minutes at 37° C. The plate is washed with 200 μl of PBS three times, and 100 μl of MBP-CnaA or MBP-CnaA/untagged CnaA mixture is added to each well for a 2-hour incubation at 37° C. After washing with 200 μl of PBS three times, 100 μl of 0.125 ug/ml of anti-His conjugated with HRP is added to each well and incubated for 1 hour at room temperature. The plate is then washed with 200 μl of PBS three times, and 100 μl of TMB substrate is added to each well and allowed to develop for 30 minutes. To stop the reaction, 50 μl of 1 M HCl is added to each well. Absorbance of the plate at 450 nm is read to quantify binding.

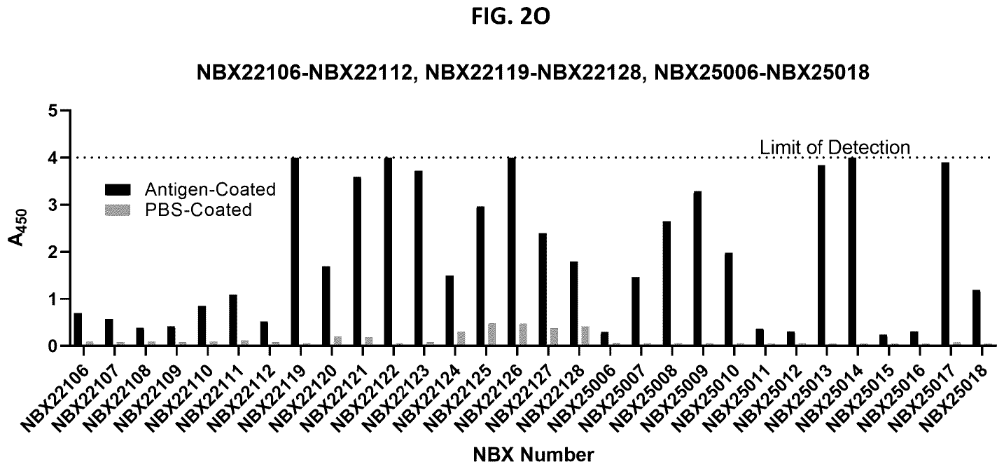

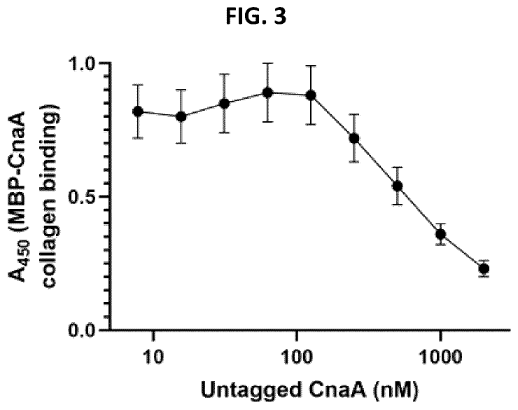

FIG. 3 shows the reduction of binding of MBP-CnaA to collagen in the presence of increasing concentrations of untagged CnaA.

### 7. NBX Stability in Chicken Jejunal Extracts

Experiments were conducted by thawing frozen chicken jejunum extract and NBX at room temperature, and immediately placing both on ice. Chicken jejunum extract and protein were spun at 10,000 RCF for 1 minute to pellet and remove any precipitation. PBS and saline were prechilled on ice. Two reactions were set up in volumes of 10 μl on ice. The first reaction contained no chicken jejunum extract and consisted of 5 μg NBX in 3.2 μL PBS and 4.8 μL of 150 mM NaCl. The second reaction contained chicken jejunum extract and was generated using the following ratios: 2.4 μL chicken jejunum extract, 5 μg NBX in 0.8 μL PBS, and 4.8 μL of 150 mM NaCl. The tubes were incubated on ice for 5 minutes, followed by 42° C. for up to 24 hours. The final incubation temperature (42° C.) is the internal temperature of a chicken. After incubation, 8 μL of preheated 2×SDS sample buffer was added to stop the reaction, and the sample was boiled at 95-100° C. for 5 minutes. The stability of each NBX was assessed by the presence or absence of the NBX on an 18% SDS-PAGE gel.

FIG. 4C shows the stability of six NBXs (NBX0324, NBX0365, NBX0880, NBX0888, NBX08108, and NBX11002) in the presence of the chicken jejunum extract.

### 8. NBX Reduction of Poultry Necrotic Enteritis

Preparation of Bacillus subtilis Secreting NBXs

Bacillus subtilis strain PY79 (obtained from the Bacillus Genetic Stock Center). Bacillus subtilis clones secreting individual NBXs were created for NBX0324 (SEQ ID 20), NBX0365 (SEQ ID 216), NBX0880 (SEQ ID 313), NBX0888 (SEQ ID 320), NBX08108 (SEQ ID 340), and NBX11002 (SEQ ID 758). NBX genes were integrated into the Bacillus subtilis genome using established protocols known in the art (^{9,10}).

### Preparation of Enteric Coated Yeast Extracts Containing NBXs

Pichia pastoris strains BG10 and BG11 and the expression vector pD912 were obtained from ATUM. NBX0324 (SEQ ID 20), NBX0365 (SEQ ID 216), NBX0880 (SEQ ID 313), NBX0888 (SEQ ID 320), NBX08108 (SEQ ID 340), and NBX11002 (SEQ ID 758) were cloned individually into pD912 and clones of P. pastoris strains BG10 and BG11 expressing each of the six NBXs were created following protocols provided by ATUM. P. pastoris strain BG10 expressing NBX0365 (SEQ ID 216) was commercially grown in a fermenter by EUCODIS Bioscience. P. pastoris strain BG11 expressing NBX0324 (SEQ ID 20), NBX0880 (SEQ ID 313), NBX0888 (SEQ ID 320), NBX08108 (SEQ ID 340), or NBX11002 (SEQ ID 758) were commercially grown in fermenters by the National Center in Environmental Technology and Electrochemistry (CNETE). For all fermentation products, the cell biomass was removed by centrifugation, the NBX containing supernatants were filtered to remove media components and other small molecules, concentrated, and freeze-dried.

The dried yeast extracts containing NBXs were mixed and encapsulated in a proprietary lipid-based matrix by Jefo Nutrition at an inclusion rate of 18%. The purpose of the matrix is to protect the NBXs through the upper gastrointestinal tract. Necrotic enteritis challenge

Five hundred sixty chicks were assigned to seven treatment groups with eight cages per treatment and ten chicks per cage. Treatment groups were assigned to cages using randomized complete block. The study began when chicks were placed (day-of-hatch; day 0), at which time chicks were allocated to experimental cages. Only healthy birds were selected. Chicks were raised in Petersime style battery cages (stocking density of 0.63 square feet per bird) in a solid-sided barn and maintained under ambient humidity. Feed and water were available ad libitum throughout the trial. Each cage contained one trough feeder and one trough drinker (10 bird to feeder/drinker ratio, 24-inch×3.5-inch trough).

All birds were weighed by pen on days 0, 14, 22, and 28. Feed added to each pen's feeder was weighed at the beginning of each formulation period on days 0 and 14. Any additional bags of feed were weighed (and documented) for each pen (as required) during each formulation period. Feed was distributed as needed to feeders from pre-weighed bags (assigned to each pen) throughout each period. Feed remaining in feeders (and feed bags if applicable) were weighed and disposed of on days 14, 22, and 28. Empty pan feeder weights were recorded prior to study initiation. The trial was terminated on day 28.

The necrotic enteritis challenge model consisted of approximately 2,000-5,000 Eimeria maxima oocysts gavaged on day 14 as the predisposing factor and Clostridium perfringens gavaged on days 19 and 20 using 1.0 mL at a concentration of 1.0×10⁸ CFU/mL per Table 6.

Table 6 describes the various treatment groups which consisted of non-challenged and untreated control (T1), challenged and untreated control (T2), challenged and antibiotic treated control (T3), challenged and treated with a cocktail of six NBXs collected as a supernatant extract from Pichia pastoris fermentation and enteric coated (T4), challenged and treated with an off-target NBX collected as supernatant extract from Pichia pastoris fermentation and enteric coated (T5), challenged and treated with Bacillus subtilis spores from six strains each secreting a different NBX (T6), and challenged and treated with Bacillus subtilis spores of a strain that does not secrete and NBX (T7).

TABLE 6
Necrotic Enteritis Challenge Experimental Design

| | | Necrotic | Dose of Test Article | Dose of Test |
|---|---|---|---|---|
| Treatment | | Enteritis | in Starter Feed | Article in Feed |
| Group | Treatment | Challenge | (days 0-18) | (days 18-28) |
| T1 | Non-challenge | No | None | None |
| | Control | | | |
| T2 | Challenge Control | Yes | None | None |
| T3 | Bacitracin methylene | Yes | None | 55 gram/metric |
| | disalicylate (BMD) | | | ton |
| T4 | Enteric coated active | Yes | None | 5.3 gram/kg |
| | NBX | | | |
| T5 | Enteric coated off- | Yes | None | 4.3 gram/kg |
| | target NBX | | | |
| T6 | Bacillus subtilis | Yes | 6 × 10⁹ CFU per kg | 6 × 10⁹ CFU per kg |
| | secreting active NBX | | | |
| T7 | Bacillus subtilis with | Yes | 6 × 10⁹ CFU per kg | 6 × 10⁹ CFU per kg |
| | no NBX secretion | | | |

The six NBXs present in treatment groups T4 and T6 are NBX0324 (SEQ ID 20), NBX0365 (SEQ ID 216), NBX0880 (SEQ ID 313), NBX0888 (SEQ ID 320), NBX08108 (SEQ ID 340), and NBX11002 (SEQ ID 758).

Table 7 indicates the effects of different treatments on necrotic enteritis mortality. Enteric coated active NBXs reduced mortality similarly to the antibiotic control. Bacillus subtilis secreting active NBX showed the greatest reduction in mortality and was the only treatment group (including the antibiotic control) where the reduction compared to treatment group T2 was statistically significant.

TABLE 7
Clinical Necrotic Enteritis Results

| Treatment | | Necrotic Enteritis | |
|---|---|---|---|
| Group | Treatment | Percent Mortality* | |
| T1 | Non-challenge Control | 0.00 | (C) |
| T2 | Challenge Control | 23.75 | (A) |
| T3 | Bacitracin methylene disalicylate | 11.25 | (A, B, C) |
| | (BMD) | | |
| T4 | Enteric coated active NBX | 11.25 | (A, B, C) |
| T5 | Enteric coated off-target NBX | 17.5 | (A, B) |
| T6 | Bacillus subtilis secreting active NBX | 7.5 | (B, C) |
| T7 | Bacillus subtilis with no NBX secretion | 15 | (A, B) |

*Values that share a letter in the brackets are not statistically significantly different based on a t-test and a significance level of p < 0.05.

Table 8 indicates the effects of treatments on chicken growth performance. Performance results were calculated as previously described (11). Treatment Group T6 (Bacillus subtilis secreting active NBX) improves the feed intake and weight gain of challenged chickens at a level comparable to the positive control antibiotic (T3, BMD). Treatment Group T6 (Bacillus subtilis secreting active NBX) provides the best improvement to challenged chickens in terms of the non-adjusted feed conversion ratio, even better than the positive control antibiotic (T3, BMD).

TABLE 8
Day 0 to Day 28 Performance Results

| | | | | Non-Adjusted | | | |
|---|---|---|---|---|---|---|---|
| Treatment | | Feed Intake | | Feed Conversion | | Weight Gain | |
| Group | Treatment | (kg/cage)* | | Ratio* | | (kg)* | |
| T1 | Non-challenge | 15.50 | (A) | 1.437 | (C) | 1.121 | (A) |
| | Control | | | | | | |
| T2 | Challenge Control | 13.11 | (D, E) | 1.972 | (A) | 1.009 | (B, C, D) |
| T3 | Bacitracin | 15.13 | (A, B) | 1.691 | (A, B, C) | 1.090 | (A, B) |
| | methylene | | | | | | |
| | disalicylate (BMD) | | | | | | |
| T4 | Enteric coated | 13.44 | (C, D, E) | 1.698 | (A, B, C) | 0.993 | (B, C, D) |
| | active NBX | | | | | | |
| T5 | Enteric coated off- | 12.31 | (E) | 1.806 | (A, B) | 0.958 | (C, D) |
| | target NBX | | | | | | |
| T6 | Bacillus subtilis | 14.48 | (A, B, C) | 1.564 | (B, C) | 1.091 | (A, B) |
| | secreting active NBX | | | | | | |
| T7 | Bacillus subtilis with | 13.89 | (B, C, D) | 1.757 | (A, B) | 1.035 | (A, B, C) |
| | no NBX secretion | | | | | | |

*Values within a given column that share a letter in the brackets are not statistically significantly different based on a t-test and a significance level of p < 0.05.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All publications, patent applications, issued patents, and other documents referred to in this specification are herein incorporated by reference as if each individual publication, patent application, issued patent, or other document is specifically and individually indicated to be incorporated by reference in its entirety. Definitions that are contained in text incorporated by reference are excluded to the extent that they contradict definitions in this disclosure.

The following references are incorporated by reference in their entirety.
1. Wade, B. & Keyburn, A. (2015). The true cost of necrotic enteritis. World Poultry, 31, pp. 16-17
2. Moore, R. J. (2016). Necrotic enteritis predisposing factors in broiler chickens. Avian Pathology, 45 (3), pp. 275-281.
3. Abid, S. A. et al. (2016). Emerging threat of necrotic enteritis in poultry and its control without use of antibiotics: a review. The Journal of Animal and Plant Sciences, 26 (6), pp. 1556-1567.
4. Prescott, J. F. et al. (2011). The pathogenesis of necrotic enteritis in chickens: what we know and what we need to know: a review. Avian Pathology, 45 (3), pp. 288-294.
5. Collier, C. T. et al. (2008) Coccidia-induced mucogenesis promotes the onset of necrotic enteritis by supporting Clostridium perfringens growth. Veterinary Immunology and Immunopathology, 122 (1-2), pp. 104-115.
6. Van Meirhaeghe, H. & De Gussem, M. (2014). Coccidiosis a major threat to the chicken gut. Poultry World.
7. Chapman, H. D. (2014). Milestones in avian coccidiosis research: a review. Poultry Science, 93 (3), pp. 501-511.
8. Shivaramaiah, S. et al. (2011). The role of an early Salmonella Typhimurium infection as a predisposing factor for necrotic enteritis in a laboratory challenge model. Avian Diseases, 55 (2), pp. 319-323.
9. Zeigler, D. R. (2002). Bacillus Genetic Stock Center; Catalog of Strains, 7^{th} Edition; Volume 4: Integration Vectors of Gram-Positive Organisms.
10. Yang, M. et al. (2020). Engineering Bacillus subtilis as a versatile and stable platform for production of nanobodies. Applied and Environmental Microbiology, 86 (8), Article e02938-19.
11. Hofacre, W. G. et al. (1998). Use of Aviguard and other intestinal bioproducts in experimental Clostridium perfrigens-associated necrotizing enteritis in broiler chickens. Avian Disease, 42 (3), pp. 579-584.

What is claimed is:

1. A polypeptide comprising at least one variable region fragment of a heavy chain antibody (V_{H}H) having an amino acid sequence at least 99% identical to any one of SEQ ID NOs: 1 to 56 or 212 to 340 or 695 to 968 or 1791 to 1795,
wherein the polypeptide is capable of a) reducing the cytotoxicity of NetB with an IC50 value less than 1 mM by at least 90%; b) reducing the binding of CnaA to collagen by greater than 50% at 2 uM; or c) reducing Cpa lecithinase activity by greater than 40% at 1 uM.

2. The polypeptide of claim 1, wherein the V_{H}H comprises:
a) a complementarity determining region 1 (CDR1) as set forth in any one of SEQ ID NOs: 57 to 106 or 341 to 458 or 969 to 1242 or 1796 to 1800,
b) a complementarity determining region 2 (CDR2) as set forth in any one of SEQ ID NOs: 107 to 156 or 459 to 576 or 1243 to 1516 or 1801 to 1805, and
c) a complementarity determining region 3 (CDR3) as set forth in any one of SEQ ID NOs: 157 to 206 or 577 to 694 or 1517 to 1790 or 1806 to 1810.

3. The polypeptide of claim 1, wherein the polypeptide comprises a plurality of V_{H}HS.

4. The polypeptide of claim 3, wherein the plurality of V_{H}Hs is identical to another V_{H}H of the plurality of V_{H}Hs.

5. The polypeptide of claim 3, wherein the plurality of V_{H}Hs comprises at least three V_{H}Hs.

6. The polypeptide of claim 3, wherein the plurality of V_{H}Hs are covalently coupled to one another by a linker, the linker comprising one or more amino acids.

7. A polypeptide complex comprising the polypeptides of claim 1, wherein the polypeptide complex comprises:
a) a first component polypeptide, and
b) a second component polypeptide,
wherein the first component polypeptide and the second component polypeptide are not covalently linked together and are coupled together by a protein-protein interaction, a small molecule-protein interaction, or a small molecule-small molecule interaction, and
wherein each of the first and the second component polypeptides comprise a V_{H}H which specifically binds a pathogen.

8. The polypeptide of claim 7, wherein the pathogen is a poultry-associated bacterium.

9. The polypeptide of claim 8, wherein the poultry-associated bacterium comprises a species of Clostridium.

10. The polypeptide of claim 9, wherein the species of Clostridium is Clostridium perfringens.

11. The polypeptide of claim 1, wherein the V_{H}H specifically binds a Clostridium virulence factor, wherein the Clostridium virulence factor is NetB polypeptide, NetB-like toxin polypeptide, Cpa polypeptide, Cpa-like toxin polypeptide, Cpb2 polypeptide, Cpb2-like toxin polypeptide, CnaA polypeptide, CnaA-like polypeptide, CnaA collagen binding domain polypeptide, or CnaA collagen binding domain-like polypeptide.

12. The polypeptide of claim 1, wherein the V_{H}H specifically binds an antigen or polypeptide at least 99% identical to SEQ ID NOs: 207, 208, 209, 210, 211 or combinations thereof.

13. The polypeptide of claim 1 further comprising a vitamin, an antibiotic, a hormone, an antimicrobial peptide, a steroid, a probiotic, a probiotic, a bacteriophage, chitin, chitosan, B-1,3-glucan, vegetable extracts, peptone, shrimp meal, krill, algae, B-cyclodextran, alginate, gum, tragacanth, pectin, gelatin, an additive spray, a toxin binder, a short chain fatty acid, a medium chain fatty acid, yeast, a yeast extract, sugar, a digestive enzyme, a digestive compound, an essential mineral, an essential salt, or fiber.

14. A vector or a cell comprising a nucleic acid or a plurality of nucleic acids encoding the polypeptide of claim 1.

15. The vector or cell of claim 14, wherein the polypeptide is produced by incubating the cell in a medium suitable for secretion of the polypeptide from the cell and purifying the polypeptide from the medium.

16. A method of of reducing the activity of a Clostridium perfringens virulence factor comprising administering to a poultry animal, a non-poultry animal species, or human individual a polypeptide at least 99% identical to SEQ IDs Nos: 207, 208, 209, 210, 211, or combinations thereof.

17. The method of claim 16, wherein the method reduces or prevents a poultry-associated bacterial infection or reduces transmission or prevents transmission of a poultry-associated bacterial from the poultry species to another poultry animal, another animal species, or human individual.

18. The method of claim 16, wherein the poultry animal is a species of a chicken, turkey, duck, quail, pigeon, squab, ostrich, or goose and the non-poultry animal species is a pig, sheep, goat, horse, cow, llama, alpaca, mink, rabbit, dog, cat, or human.

19. The method of claim 16, wherein the polypeptide is adapted for introduction to the alimentary canal orally or rectally, provided to the exterior surface (for example, as a spray or submersion), provided to the medium in which the animal dwells (including air based media), provided by injection, provided intravenously, provided via the respiratory system, provided via diffusion, provided via absorption by the endothelium or epithelium, or provided via a secondary organism such as a yeast, bacterium, algae, bacteriophages, plants and insects to a host.

20. The method of claim 16, wherein the polypeptides of SEQ ID NOs: 210 or 211 can outcompete Clostridium perfringens surface-expressed CnaA binding to collagen by 10%, 20%, 30%, 40%, 50%, 60%, 70%, 80%, 90%, 95%, 99%, or 100%.
